(19) United States
(12) Patent Application Publication
(10) Pub. No.: US 2006/0178515 A1
(43) Pub. Date: Aug. 10, 2006

(54) Tetrahydroisoquinolyl acetamide derivatives for use as orexin receptor antagonists

(76) Inventors: Hamed Aissaoui, PULVERSHEIM (FR); Martine Clozel, Binningen (CH); Thomas Weller, Binningen (CH); Ralf Koberstein, Lorrach (DE); Thierry Sifferlen, Guewenheim (FR); Walter Fischli, Allschwil (CH)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/549,180

(22) PCT Filed: Mar. 23, 2004
(86) PCT No.: PCT/EP04/03057

§ 371 Date: Sep. 16, 2005

(30) Foreign Application Priority Data
Mar. 26, 2003 (WO) PCT/EP03/03143

(51) Int. Cl. C07D 217/12

(52) U.S. Cl. 546/146

(57) ABSTRACT

The invention relates to novel acetamide derivatives of formula (I) and their use as active ingredients in the preparation of pharmaceutical compositions. The invention also concerns related aspects including processes for the preparation of such compounds, pharmaceutical compositions containing one or more of those compounds and especially their use as orexin receptor antagonists.

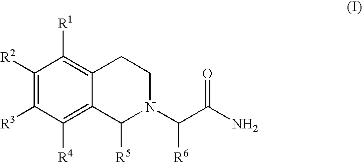

The present invention relates to novel acetamide derivatives of formula (I) and their use as pharmaceuticals. The invention also concerns related aspects including processes for the preparation of the compounds, pharmaceutical compositions containing one or more compounds of formula (I), and especially their use as orexin receptor antagonists. The orexins (hypocretins) comprise two neuropeptides produced in the hypothalamus: the orexin A (OX-A) (a 33 aminoacid peptide) and the orexin B (OX-B) (a 28 aminoacid peptide) (Sakurai T. et al., Cell, 1998, 92, 573-585). Orexins are found to stimulate food consumption in rats suggesting a physiological role for these peptides as mediators in the central feedback mechanism that regulates feeding behaviour (Sakurai T. et al., Cell, 1998, 92, 573-585). On the other hand, it was also proposed that orexins regulate states of sleep and wakefulness opening potentially novel therapeutic approaches for narcoleptic patients (Chemelli R. M. et al., Cell, 1999, 98, 437-451). Two orexin receptors have been cloned and characterised in mammals which belong to the G-protein coupled receptor superfamily (Sakurai T. et al., Cell, 1998, 92, 573-585), the orexin-1 receptor (OX₁) which is selective for OX-A and the orexin-2 receptor (OX₂) which is capable to bind OX-A as well as OX-B.

Orexin receptors are found in the mammalian brain and may have numerous implications in pathologies such as depression; anxiety; addictions, obsessive compulsive disorder; affective neurosis; depressive neurosis; anxiety neurosis; dysthymic disorder; mood disorder; sexual dysfunction; psychosexual dysfunction; sex disorder; schizophrenia; manic depression; delirium; dementia; severe mental retardation and dyskinesias such as Huntington's disease and Tourette syndrome; eating disorders; sleep disorders; cardiovascular diseases, diabetes; appetite/taste disorders; vomiting/nausea; asthma; Parkinson's disease; Cushing's syndrome/disease; basophil adenoma; prolactinoma; hyperprolactinemia; hypopituitarism; hypophysis tumour/adenoma; hypothalamic diseases; inflammatory bowel disease; gastric dyskinesia; gastric ulcers; Froehlich's syndrome; hypophysis diseases, hypothalamic hypogonadism; Kallman's syndrome (anosmia, hyposmia); functional or psychogenic amenorrhea; hypothalamic hypothyroidism; hypothalamic-adrenal dysfunction; idiopathic hyperprolactinemia; hypothalamic disorders of growth hormone deficiency; idiopathic growth deficiency; dwarfism; gigantism; acromegaly; disturbed biological and circadian rhythms; sleep disturbances associated with diseases such as neurological disorders, neuropathic pain and restless leg syndrome; heart and lung diseases, acute and congestive heart failure; hypotension; hypertension; urinary retention; osteoporosis; angina pectoris; myocardial infarction; ischemic or haemorrhagic stroke; subarachnoid haemorrhage; ulcers; allergies; benign prostatic hypertrophy; chronic renal failure; renal disease; impaired glucose tolerance; migraine; hyperalgesia; pain; enhanced or exaggerated sensitivity to pain such as hyperalgesia, causalgia, and allodynia; acute pain; burn pain; atypical facial pain; neuropathic pain; back pain; complex regional pain syndrome I and II; arthritic pain; sports injury pain; pain related to infection e.g. HIV, post-chemotherapy pain; post-stroke pain; post-operative pain; neuralgia; conditions associated with visceral pain such as irritable bowel syndrome, migraine and angina; urinary bladder incontinence e.g. urge incontinence; tolerance to narcotics or withdrawal from narcotics; sleep apnea; narcolepsy; insomnia; parasomnia; and neurodegenerative disorders including nosological entities such as disinhibition-dementia-parkinsonism-amyotrophy complex; pallido-ponto-nigral degeneration epilepsy; seizure disorders and other diseases related to general orexin system dysfunction.

Up to now some low molecular weight compounds are known which have a potential to antagonise either specifically OX₁ or OX₂, or both receptors at the same time. In WO 99/09024, WO 99/58533, WO 00/47576, WO 00/47577 and WO 00/47580 formerly SmithKline Beecham reported phenylurea, phenylthiourea and cinnamide derivatives as OX₁ selective antagonists. More recently WO 01/85693 from Banyu Pharmaceuticals has been published wherein N-acyltetrahydroisoquinoline derivatives are disclosed. 2-Aminomethylpiperidine derivatives (WO 01/96302), 3-aminomethyl-morpholine derivatives (WO 02/44172) and N-aroyl cyclic amines (WO 02/89800, WO 02/90355, WO 03/51368 and WO 03/51871) have been suggested by formerly SmithKline Beecham as orexin receptor antagonists. Related compounds are disclosed in WO 03/02559, WO 03/02561, WO 03/32991, WO 03/41711, WO 03/51872 and WO 03/51873. In WO 03/37847 formerly SmithKline Beecham reported benzamide derivatives as orexin receptor antagonists. International patent applications WO 01/68609 and WO 02/51838 disclose 1,2,3,4-tetrahydroisoquinoline and novel benzazepine derivatives as orexin receptor antagonists. The novel compounds of the present invention belong to a different class of low molecular weight compounds as compared to all prior art orexin receptor antagonists so far published.

The present invention comprises acetamide derivatives, which are non-peptide antagonists of the human orexin receptors. These compounds, therefore, are of potential use in the treatment of disturbed homeostasis and eating disorders (e.g. bulimia, obesity, food abuse, compulsive eating or irritable bowel syndrome), as well as disturbed sleep/wake schedule, sleep disorders (e.g. insomnias, apneas, dystonias) or stress-related diseases (e.g. anxiety, mood and blood pressure disorders) or any other disease related to the orexin dysfunction.

The present invention relates to novel acetamide derivatives of the formula (I). 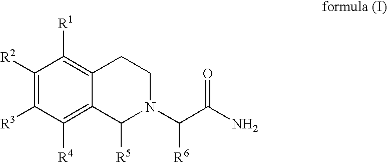
wherein:
R¹, R², R³, R⁴ independently represent hydrogen; cyano; halogen; hydroxyl; lower alkyl; lower alkenyl; lower alkoxy; lower alkenyloxy; trifluoromethoxy; cycloalkyloxy or R¹ and R² together as well as R² and R³ together or R³ and R⁴ together may form with the phenyl ring, to which they are attached, a five, six or seven-membered ring containing one or two oxygen atoms;
R⁵ represents unsubstituted phenyl-C1-C4 alkyl or naphthyl-C1-C4 alkyl; mono-, di- or tri-substituted phenyl-C1-C4 alkyl whereby the substituents independently are lower alkyl, lower alkoxy, lower alkenyl, trifluoromethyl, trifluormethoxy, difluormethoxy or halogen; unsubstituted or mono-, di- or tri-substituted phenyl-C2-C4 alkenyl whereby the substituents independently are lower alkyl, lower alkoxy, lower alkenyl, trifluoromethyl, trifluormethoxy, difluormethoxy or halogen; a five- or six-membered heterocyclic ring-C1-C4 alkyl, whereby these rings contain one oxygen, one nitrogen or one sulphur atom; or two hetero atoms selected independently from oxygen, nitrogen or sulphur, whereby these heterocyclic rings are unsubstituted or mono- di- or tri-substituted independently with lower alkyl, lower alkoxy, lower alkenyl, trifluoromethyl, trifluormethoxy, difluormethoxy or halogen; cycloalkyl-C1-C4 alkyl; or R⁷OCH₂—;
R⁶ represents hydrogen, phenyl; mono-, di- or tri-substituted phenyl whereby the substituents independently are lower alkyl, lower alkoxy, lower alkenyl, trifluoromethyl or halogen;
R⁷ represents C1-C4 alkyl, phenyl; mono-, di- or tri-substituted phenyl whereby the substituents independently are C1-C4 alkyl, C1-C4 alkoxy, C2-C4 alkenyl, trifluoromethyl, trifluormethoxy, difluormethoxy or halogen; phenyl-C1-C4 alkyl; mono-, di- or tri-substituted phenyl-C1-C4 alkyl, whereby the substituents independently are C1-C4 alkyl, C1-C4 alkoxy, C2-C4 alkenyl, trifluoromethyl, trifluormethoxy, difluormethoxy or halogen;
and pure enantiomers, mixtures of enantiomers, pure diastereoisomers, mixtures of diastereoisomers, diastereoisomeric racemates, mixtures of diastereoisomeric racemates and the meso-form and pharmaceutically acceptable salts, solvent complexes, and morphological forms, thereof.

In the present description the term “lower alkyl”, alone or in combination, means a straight-chain or branched-chain alkyl group with 1 to 8 carbon atoms, preferably a straight or branched-chain alkyl group with 1-4 carbon atoms. Examples of straight-chain and branched C₁-C₈ alkyl groups are methyl, ethyl, propyl, isopropyl, butyl, pentyl, hexyl, heptyl, octyl, isobutyl, sec.-butyl, tert.-butyl, the isomeric pentyls, the isomeric hexyls, the isomeric heptyls and the isomeric octyls, preferably methyl, ethyl, propyl, isopropyl, butyl, sec.-butyl, tert.-butyl, isobutyl and pentyl.

The term “lower alkenyl”, alone or in combination, means a straight-chain or branched-chain alkenyl group with 2 to 5 carbon atoms, preferably 2 to 4 carbon atoms, preferably allyl and vinyl.

The term “lower alkoxy”, alone or in combination, means a group of the formula alkyl-O— in which the term “alkyl” has the previously given significance, such as methoxy, ethoxy, n-propoxy, isopropoxy, n-butoxy, isobutoxy, sec.-butoxy and tert.-butoxy, preferably methoxy and ethoxy.

Lower alkenyloxy groups are preferably vinyloxy and allyloxy.

The term “cycloalkyl”, alone or in combination, means a cycloalkyl ring with 3 to 8 carbon atoms and preferably a cycloalkyl ring with 3 to 6 carbon atoms. Examples of C₃-C₈ cycloalkyl are cyclopropyl, cyclobutyl, cyclopentyl, cyclohexyl, cycloheptyl and cyclooctyl, preferably cyclopropyl, cyclohexyl and particularly cyclohexyl or lower alkyl substituted cycloalkyl which may preferably be substituted with lower alkyl such as methyl-cyclopropyl, dimethyl-cyclopropyl, methyl-cyclobutyl, methyl-cyclopentyl, methyl-cyclohexyl, dimethyl-cyclohexyl.

The term “aryl”, alone or in combination, means a phenyl or naphthyl group which optionally carries one or more substituents, preferably one or two substituents, each independently selected from cyano, halogen, hydroxy, lower alkyl, lower alkenyl, lower alkoxy, lower alkenyloxy, nitro, trifluoromethyl, difluoromethoxy, trifluoromethoxy, NH₂CO—, CH₃NHCO—, (CH₃)₂NCO—, H₂N—, CH₃NH—, (CH₃)₂N—, CH₃SO₂NH—, CH₃NHSO₂— or CH₃CO—NH—. Preferred are naphthyl, phenyl and phenyl independently substituted with lower alkyl, lower alkoxy, trifluoromethyl, difluoromethoxy, trifluoromethoxy, or halogen.

The term “aralkyl”, alone or in combination, means a lower alkyl or cycloalkyl group as previously defined in which one hydrogen atom has been replaced by an aryl group as previously defined. Preferred are benzyl or 2-phenyl-ethyl groups that might be unsubstituted or independently substituted at the aryl group with lower alkyl, lower alkoxy, trifluoromethyl, difluoromethoxy, trifluoromethoxy or halogen.

The term “aralkenyl”, alone or in combination, means a lower alkenyl group as previously defined in which one hydrogen atom has been replaced by an aryl group as previously defined. Preferred are 2-phenyl-ethenyl groups. Particularly preferred are 2-phenyl-ethenyl groups substituted at the phenyl group with one or two fluorine atoms.

For the term “heterocyclyl” and “heterocyclyl-lower alkyl”, the heterocyclyl group is preferably a 5- to 10-membered monocyclic or bicyclic ring, which may be saturated, partially unsaturated or aromatic containing for example 1, 2 or 3 heteroatoms selected from oxygen, nitrogen and sulphur which may be the same or different. Examples of such heterocyclyl groups are pyrrolidinyl, piperidinyl, piperazinyl, morpholinyl, pyridyl, pyrimidinyl, pyrazinyl, pyridazinyl, quinolyl, isoquinolyl, thienyl, thiazolyl, isothiazolyl, furyl, imidazoyl, pyrazolyl, pyrrolyl, indazolyl, indolyl, isoindolyl, isoxazolyl, oxazolyl, quinoxalinyl, phthalazinyl, cinnolinyl, dihydropyrrolyl, pyrrolidinyl, isobenzofuranyl, tetrahydrofuranyl, dihydropyranyl. The heterocyclyl group, if aromatic, may have up to 3 optional substituents. Saturated heterocyclyl groups may have one substituent. Examples of suitable substituents include halogen, lower alkyl, amino, nitro, cyano, hydroxy, lower alkoxy, carboxy and lower alkyloxy-carbonyls.

The term “halogen” means fluorine, chlorine, bromine or iodine and preferably fluorine and chlorine.

The term “carboxy”, alone or in combination, signifies a —COOH group.

Examples of preferred compounds of formula (I) are:

2-(6,7-Dimethoxy-1-naphthalen-2-ylmethyl-3,4-dihydro-1H-isoquinolin-2-yl)-2-phenyl-acetamide;
2-{1-[2-(3,4-Difluoro-phenyl)-ethyl]-6,7-dimethoxy-3,4-dihydro-H-isoquinolin-2-yl-2-phenyl-acetamide;
2-[1-(2-Furan-2-yl-ethyl)-6,7-dimethoxy-3,4-dihydro-1H-isoquinolin-2-yl]-2-phenyl-acetamide;
2-{1-[2-(2,3-Difluoro-phenyl)-ethyl]-6,7-dimethoxy-3,4-dihydro-1H-isoquinolin-2-yl}-2-phenyl-acetamide;
2-[1-(2,5-Dimethoxy-benzyl)-6,7-dimethoxy-3,4-dihydro-1H-isoquinolin-2-yl]-2-phenyl-acetamide;
2-{6,7-Dimethoxy-1 [2-(4-trifluoromethyl-phenyl)-ethyl]-3,4-dihydro-1H-isoquinolin-2-yl}-2-phenyl-acetamide;
2-{6,7-Dimethoxy-1-[2-(3-methoxy-phenyl)-ethyl]-3,4-dihydro-1H-isoquinolin-2-yl}-2-phenyl-acetamide;
2-[6,7-Dimethoxy-1-(4-methoxy-benzyl)-3,4-dihydro-1H-isoquinolin-2-yl]-2-phenyl-acetamide;
[2-(2,5-Difluoro-phenyl)-ethyl]-6,7-dimethoxy-3,4-dihydro-1H-isoquinolin-2-yl-2-phenyl-acetamide;
2-{6,7-Dimethoxy-1-[2-(2-methoxy-phenyl)-ethyl]-3,4-dihydro-1H-isoquinolin-2-yl}-2-phenyl-acetamide;
2-{1-[2-(4-Fluoro-phenyl)-ethyl]-6,7-dimethoxy-3,4-dihydro-1H-isoquinolin-2-yl}-2-phenyl-acetamide;
2-[1-(3-Fluoro-4-methoxy-benzyl)-6,7-dimethoxy-3,4-dihydro-1H-isoquinolin-2-yl]-2-phenyl-acetamide;
2-{1-[2-(3,4-Dimethoxy-phenyl)-ethyl]-6,7-dimethoxy-3,4-dihydro-1H-isoquinolin-2-yl}-2-phenyl-acetamide;
2-(6,7-Dimethoxy-1-phenoxymethyl-3,4-dihydro-1H-isoquinolin-2-yl)-2-phenyl-acetamide;
2-[6,7-Dimethoxy-1-(3-methoxy-benzyl)-3,4-dihydro-1H-isoquinolin-2-yl]-2-phenyl-acetamide;
2-[6-(3,4-Dimethoxy-benzyl)-2,3,8,9-tetrahydro-6H-[1,4]dioxino[2,3-g]isoquinolin-7-yl]-2-phenyl-acetamide;
2-[6,7-Dimethoxy-1-(3-phenyl-propyl)-3,4-dihydro-1H-isoquinolin-2-yl]-2-phenyl-acetamide;
(R)-2-{(S)-6,7-Dimethoxy-1-[2-(2-methyl-5-trifluoromethyl-phenyl)-ethyl]-3,4-dihydro-1H-isoquinolin-2-yl}-2-phenyl-acetamide;
(R)-2-{(S)-1-[2-(3-Chloro-2-fluoro-phenyl)-ethyl]-6,7-dimethoxy-3,4-dihydro-1H-isoquinolin-2-yl}-2-phenyl-acetamide;
(R)-2-{(S)-1-[2-(3-Fluoro-4-methyl-phenyl)-ethyl]-6,7-dimethoxy-3,4-dihydro-1H-isoquinolin-2-yl}-2-phenyl-acetamide;
(R)-2-{(S)-1-[2-(4-Fluoro-2-trifluoromethyl-phenyl)-ethyl]-6,7-dimethoxy-3,4-dihydro-1H-isoquinolin-2-yl}-2-phenyl-acetamide;
(R)-2-{(S)-1-[2-(5-Fluoro-2-trifluoromethyl-phenyl)-ethyl]-6,7-dimethoxy-3,4-dihydro-1H-isoquinolin-2-yl}-2-phenyl-acetamide;
(R)-2-{(S)-1-[2-(2-Fluoro-5-trifluoromethyl-phenyl)-ethyl]-6,7-dimethoxy-3,4-dihydro-1H-isoquinolin-2-yl}-2-phenyl-acetamide;
(R)-2-{(S)-1-[2-(3-Fluoro-4-trifluoromethyl-phenyl)-ethyl]-6,7-dimethoxy-3,4-dihydro-1H-isoquinolin-2-yl}-2-phenyl-acetamide;
(R)-2-{(S)-1-[2-(4-Fluoro-3-methyl-phenyl)-ethyl]-6,7-dimethoxy-3,4-dihydro-1H-isoquinolin-2-yl}-2-phenyl-acetamide;
(R)-2-{(S)-6,7-Dimethoxy-1-[2-(2,3,5-trifluoro-phenyl)-ethyl]-3,4-dihydro-1H-isoquinolin-2-yl}-2-phenyl-acetamide;
(R)-2-{(S)-1-[2-(4-Fluoro-3-trifluoromethyl-phenyl)-ethyl]-6,7-dimethoxy-3,4-dihydro-1H-isoquinolin-2-yl}-2-phenyl-acetamide;
(R)-2-{(S)-1-[2-(3-Fluoro-5-trifluoromethyl-phenyl)-ethyl]-6,7-dimethoxy-3,4-dihydro-1H-isoquinolin-2-yl}-2-phenyl-acetamide;
(R)-2-{(S)-1-[2-(5-Chloro-2-trifluoromethyl-phenyl)-ethyl]-6,7-dimethoxy-3,4-dihydro-1H-isoquinolin-2-yl}-2-phenyl-acetamide;
(R)-2-{(S)-1-[2-(2-Fluoro-3-trifluoromethyl-phenyl)-ethyl]-6,7-dimethoxy-3,4-dihydro-1H-isoquinolin-2-yl}-2-phenyl-acetamide;
(R)-2-{(S)-1-[2-(2-Fluoro-4-trifluoromethyl-phenyl)-ethyl]-6,7-dimethoxy-3,4-dihydro-1H-isoquinolin-2-yl}-2-phenyl-acetamide;
(R)-2-{(S)-1-[2-(2-Difluoromethoxy-phenyl)-ethyl]-6,7-dimethoxy-3,4-dihydro-1H-isoquinolin-2-yl}-2-phenyl-acetamide;
(R)-2-{(S)-1-[2-(3-Fluoro-2-trifluoromethyl-phenyl)-ethyl]-6,7-dimethoxy-3,4-dihydro-1H-isoquinolin-2-yl}-2-phenyl-acetamide;
(R)-2-{(S)-1-[2-(2-Chloro-3-trifluoromethyl-phenyl)-ethyl]-6,7-dimethoxy-3,4-dihydro-1H-isoquinolin-2-yl}-2-phenyl-acetamide;
(R)-2-{(S)-1-[2-(2-Fluoro-6-trifluoromethyl-phenyl)-ethyl]-6,7-dimethoxy-3,4-dihydro-1H-isoquinolin-2-yl}-2-phenyl-acetamide;
(R)-2-{(S)-1-[2-(4-Chloro-3-trifluoromethyl-phenyl)-ethyl]-6,7-dimethoxy-3,4-dihydro-1H-isoquinolin-2-yl}-2-phenyl-acetamide;
(R)-2-{(S)-1-[2-(2,3-Difluoro-4-methyl-phenyl)-ethyl]-6,7-dimethoxy-3,4-dihydro-1H-isoquinolin-2-yl}-2-phenyl-acetamide;
(R)-2-{(S)-1-[2-(4-Difluoromethoxy-phenyl)-ethyl]-6,7-dimethoxy-3,4-dihydro-1H-isoquinolin-2-yl}-2-phenyl-acetamide;
(R)-2-{(S)-1-[2-(3,4-Dimethyl-phenyl)-ethyl]-6,7-dimethoxy-3,4-dihydro-1H-isoquinolin-2-yl}-2-phenyl-acetamide;
(R)-2-{(S)-1-[2-(3-Chloro-4-methyl-phenyl)-ethyl]-6,7-dimethoxy-3,4-dihydro-1H-isoquinolin-2-yl}-2-phenyl-acetamide;
(R)-2-{(S)-1-[2-(2-Chloro-6-fluoro-phenyl)-ethyl]-6,7-dimethoxy-3,4-dihydro-1H-isoquinolin-2-yl}-2-phenyl-acetamide;
(R)-2-[(S)-6,7-Dimethoxy-1-(2-o-tolyl-ethyl)-3,4-dihydro-1H-isoquinolin-2-yl]-2-phenyl-acetamide;
(R)-2-[(S)-6,7-Dimethoxy-1-(2-m-tolyl-ethyl)-3,4-dihydro-1H-isoquinolin-2-yl]-2-phenyl-acetamide;
(R)-2-[(S)-6,7-Dimethoxy-1-(2-p-tolyl-ethyl)-3,4-dihydro-1H-isoquinolin-2-yl]-2-phenyl-acetamide;
(R)-2-{(S)-1-[2-(2-Fluoro-phenyl)-ethyl]-6,7-dimethoxy-3,4-dihydro-H-isoquinolin-2-yl}-2-phenyl-acetamide;
(R)-2-{(S)-1-[2-(3-Fluoro-phenyl)-ethyl]-6,7-dimethoxy-3,4-dihydro-1H-isoquinolin-2-yl}-2-phenyl-acetamide;
(R)-2-{(S)-1-[2-(3,4-Dichloro-phenyl)-ethyl]-6,7-dimethoxy-3,4-dihydro-1H-isoquinolin-2-yl}-2-phenyl-acetamide;
(R)-2-{(S)-1-[2-(3,5-Dimethyl-phenyl)-ethyl]-6,7-dimethoxy-3,4-dihydro-H-isoquinolin-2-yl}-2-phenyl-acetamide;
(R)-2-{(S)-6,7-Dimethoxy-1-[2-(2-trifluoromethyl-phenyl)-ethyl]-3,4-dihydro-1H-isoquinolin-2-yl}-2-phenyl-acetamide;
(R)-2-{(S)-1-[2-(2,4-Difluoro-phenyl)-ethyl]-6,7-dimethoxy-3,4-dihydro-1H-isoquinolin-2-yl}-2-phenyl-acetamide;
(R)-2-{(S)-6,7-Dimethoxy-1-[2-(2,3,6-trifluoro-phenyl)-ethyl]-3,4-dihydro-1H-isoquinolin-2-yl}-2-phenyl-acetamide;
(R)-2-{(S)-6,7-Dimethoxy-1-[2-(4-trifluoromethoxy-phenyl)-ethyl]-3,4-dihydro-1H-isoquinolin-2-yl}-2-phenyl-acetamide;
(R)-2-{(S)-1-[2-(2-Fluoro-3-methyl-phenyl)-ethyl]-6,7-dimethoxy-3,4-dihydro-1H-isoquinolin-2-yl}-2-phenyl-acetamide;
(R)-2-{(S)-1-[2-(4-Chloro-phenyl)-ethyl]-6,7-dimethoxy-3,4-dihydro-1H-isoquinolin-2-yl}-2-phenyl-acetamide;
(R)-2-{(S)-1-[2-(3-Chloro-phenyl)-ethyl]-6,7-dimethoxy-3,4-dihydro-1H-isoquinolin-2-yl}-2-phenyl-acetamide;
(R)-2-{(S)-6,7-Dimethoxy-1-[2-(3-trifluoromethoxy-phenyl)-ethyl]-3,4-dihydro-1H-isoquinolin-2-yl}-2-phenyl-acetamide;
(R)-2-{(S)-6,7-Dimethoxy-1-[2-(3,4,5-trifluoro-phenyl)-ethyl]-3,4-dihydro-1H-isoquinolin-2-yl}-2-phenyl-acetamide;
(R)-2-{(S)-6,7-Dimethoxy-1-[2-(3-trifluoromethyl-phenyl)-ethyl]-3,4-dihydro-1H-isoquinolin-2-yl}-2-phenyl-acetamide;
(R)-2-{(S)-6,7-Dimethoxy-1-[2-(2,3,4-trifluoro-phenyl)-ethyl]-3,4-dihydro-1H-isoquinolin-2-yl}-2-phenyl-acetamide;
(R)-2-{(S)-1-[2-(4-Bromo-2-fluoro-phenyl)-ethyl]-6,7-dimethoxy-3,4-dihydro-1H-isoquinolin-2-yl}-2-phenyl-acetamide;
(R)-2-{(S)-1-[2-(2,6-Difluoro-phenyl)-ethyl]-6,7-dimethoxy-3,4-dihydro-H-isoquinolin-2-yl}-2-phenyl-acetamide;
(R)-2-{(S)-6,7-Dimethoxy-1-[2-(2-trifluoromethoxy-phenyl)-ethyl]-3,4-dihydro-1H-isoquinolin-2-yl}-2-phenyl-acetamide;
(R)-2-{(S)-1-[2-(2,4-Dichloro-phenyl)-ethyl]-6,7-dimethoxy-3,4-dihydro-1H-isoquinolin-2-yl}-2-phenyl-acetamide;
(R)-2-{(S)-6,7-Dimethoxy-1-[2-(2,4,5-trifluoro-phenyl)-ethyl]-3,4-dihydro-1H-isoquinolin-2-yl}-2-phenyl-acetamide;
(R)-2-{(S)-1-[2-(3-Bromo-phenyl)-ethyl]-6,7-dimethoxy-3,4-dihydro-1H-isoquinolin-2-yl}-2-phenyl-acetamide;
(R)-2-{(S)-1-[2-(2-Bromo-phenyl)-ethyl]-6,7-dimethoxy-3,4-dihydro-1H-isoquinolin-2-yl}-2-phenyl-acetamide;
(R)-2-{(S)-1-[2-(4-Bromo-phenyl)-ethyl]-6,7-dimethoxy-3,4-dihydro-1H-isoquinolin-2-yl}-2-phenyl-acetamide;
(R)-2-{(S)-6,7-Dimethoxy-1-[2-(4-trifluoromethyl-phenyl)-ethyl]-3,4-dihydro-1H-isoquinolin-2-yl}-2-phenyl-acetamide;
   Examples of particularly preferred compounds of formula (I) are:
(R)-2-{(S)-1-[2-(3-Chloro-2-fluoro-phenyl)-ethyl]-6,7-dimethoxy-3,4-dihydro-1H-isoquinolin-2-yl}-2-phenyl-acetamide;
(R)-2-{(S)-1-[2-(3-Fluoro-4-methyl-phenyl)-ethyl]-6,7-dimethoxy-3,4-dihydro-1H-isoquinolin-2-yl}-2-phenyl-acetamide;
(R)-2-{(S)-6,7-Dimethoxy-1-[2-(2,3,5-trifluoro-phenyl)-ethyl]-3,4-dihydro-1H-isoquinolin-2-yl}-2-phenyl-acetamide;
(R)-2-{(S)-1-[2-(2-Difluoromethoxy-phenyl)-ethyl]-6,7-dimethoxy-3,4-dihydro-1H-isoquinolin-2-yl}-2-phenyl-acetamide;
(R)-2-{(S)-1-[2-(4-Difluoromethoxy-phenyl)-ethyl]-6,7-dimethoxy-3,4-dihydro-1H-isoquinolin-2-yl}-2-phenyl-acetamide;
(R)-2-{(S)-1-[2-(3,4-Dimethyl-phenyl)-ethyl]-6,7-dimethoxy-3,4-dihydro-1H-isoquinolin-2-yl}-2-phenyl-acetamide;
(R)-2-[(S)-6,7-Dimethoxy-1-(2-o-tolyl-ethyl)-3,4-dihydro-1H-isoquinolin-2-yl]-2-phenyl-acetamide;
(R)-2-[(S)-6,7-Dimethoxy-1-(2-m-tolyl-ethyl)-3,4-dihydro-1H-isoquinolin-2-yl]-2-phenyl-acetamide;
(R)-2-[(S)-6,7-Dimethoxy-1-(2-p-tolyl-ethyl)-3,4-dihydro-1H-isoquinolin-2-yl]-2-phenyl-acetamide;
(R)-2-{(S)-1-[2-(2-Fluoro-phenyl)-ethyl]-6,7-dimethoxy-3,4-dihydro-1H-isoquinolin-2-yl}-2-phenyl-acetamide;
(R)-2-{(S)-1-[2-(3-Fluoro-phenyl)-ethyl]-6,7-dimethoxy-3,4-dihydro-1H-isoquinolin-2-yl}-2-phenyl-acetamide;
(R)-2-{(S)-1-[2-(2,4-Difluoro-phenyl)-ethyl]-6,7-dimethoxy-3,4-dihydro-1H-isoquinolin-2-yl}-2-phenyl-acetamide;
(R)-2-{(S)-6,7-Dimethoxy-1-[2-(2,3,6-trifluoro-phenyl)-ethyl]-3,4-dihydro-1H-isoquinolin-2-yl}-2-phenyl-acetamide;
(R)-2-{(S)-1-[2-(2-Fluoro-3-methyl-phenyl)-ethyl]-6,7-dimethoxy-3,4-dihydro-1H-isoquinolin-2-yl}-2-phenyl-acetamide;
(R)-2-{(S)-6,7-Dimethoxy-1-[2-(2,3,4-trifluoro-phenyl)-ethyl]-3,4-dihydro-1H-isoquinolin-2-yl}-2-phenyl-acetamide;
(R)-2-{(S)-6,7-Dimethoxy-1-[2-(2,4,5-trifluoro-phenyl)-ethyl]-3,4-dihydro-1H-isoquinolin-2-yl}-2-phenyl-acetamide;
(R)-2-{(S)-1-[2-(3-Bromo-phenyl)-ethyl]-6,7-dimethoxy-3,4-dihydro-1H-isoquinolin-2-yl}-2-phenyl-acetamide.

The present invention encompasses physiologically usable or pharmaceutically acceptable salts of compounds of formula (I). This encompasses salts with physiologically compatible mineral acids such as hydrochloric acid, sulphuric or phosphoric acid; or with organic acids such as formic acid, methanesulphonic acid, acetic acid, trifluoroacetic acid, citric acid, fumaric acid, maleic acid, tartaric acid, succinic acid or salicylic acid and the like. The compounds of formula (I) which are acidic can also form salts with physiologically compatible bases. Examples of such salts are alkali metal, alkali earth metal, ammonium and alkylammonium salts such as Na, K, Ca or tetraalkylammonium salt. For a comprehensive list see “Handbook of Pharmaceutical Salts”, P. H. Stahl, C. G. Wermuth Eds., Wiley-VCH, Weinheim/Zürich 2002, p. 329-350. The compounds of formula (I) can also be present in the form of a zwitterion.

The present invention encompasses different solvation complexes of compounds of general formula (I). The solvation can be effected in the course of the manufacturing process or can take place separately, e.g. as a consequence of hygroscopic properties of an initially anhydrous compound of general formula (I).

The present invention further encompasses different morphological forms, e.g. crystalline forms, of compounds of general formula (I) and their salts and solvation complexes. Particular heteromorphs may exhibit different dissolution properties, stability profiles, and the like, and are all included in the scope of the present invention.

The compounds of formula (I) might have one or several asymmetric centres and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates and the meso-forms.

Preferred compounds of formula (I) have IC₅₀ values below 100 nM, particularly preferred compounds have IC₅₀ values below 10 nM which have been determined with the FLIPR (Fluorometric Imaging Plates Reader) method described in the beginning of the experimental section.

The compounds of formula (I) and their pharmaceutically usable salts are useful in the preparation of a medicament for the prevention or treatment of diseases selected from the group consisting of depression; anxiety; addictions; obsessive compulsive disorder; affective neurosis; depressive neurosis; anxiety neurosis; dysthymic disorder; mood disorder; sexual dysfunction; psychosexual dysfunction; schizophrenia; manic depression; delirium; dementia; severe mental retardation and dyskinesias such as Huntington's disease and Tourette syndrome; diabetes; appetite/taste disorders; vomiting/nausea; asthma; Parkinson's disease; Cushing's syndrome/disease; basophil adenoma; prolactinoma; hyperprolactinemia; hypopituitarism; hypophysis tumour/adenoma; hypothalamic diseases; inflammatory bowel disease; gastric dyskinesia; gastric ulcers; Froehlich's syndrome; hypophysis diseases, hypothalamic hypogonadism; Kallman's syndrome (anosmia, hyposmia); functional or psychogenic amenorrhea; hypothalamic hypothyroidism; hypothalamic-adrenal dysfunction; idiopathic hyperprolactinemia; hypothalamic disorders of growth hormone deficiency; idiopathic growth deficiency; dwarfism; gigantism; acromegaly; disturbed biological and circadian rhythms; sleep disturbances associated with diseases such as neurological disorders, neuropathic pain and restless leg syndrome; heart and lung diseases, acute and congestive heart failure; hypotension; hypertension; urinary retention; osteoporosis; angina pectoris; myocardial infarction; ischemic or haemorrhagic stroke; subarachnoid haemorrhage; ulcers; allergies; benign prostatic hypertrophy; chronic renal failure; renal disease; impaired glucose tolerance; migraine; pain; enhanced or exaggerated sensitivity to pain such as hyperalgesia, causalgia, and allodynia; acute pain; burn pain; atypical facial pain; neuropathic pain; back pain; complex regional pain syndrome I and II; arthritic pain; sports injury pain; pain related to infection e.g. by HIV; post-chemotherapy pain; post-stroke pain; post-operative pain; neuralgia; conditions associated with visceral pain such as irritable bowel syndrome; migraine and angina; urinary bladder incontinence e.g. urge incontinence; tolerance to narcotics or withdrawal from narcotics; sleep disorders; eating disorders; cardiovascular disorders; neurodegenerative disorders; sleep apnea; narcolepsy; insomnia; parasomnia; and neurodegenerative disorders including nosological entities such as disinhibition-dementia-parkinsonism-amyotrophy complex; pallido-ponto-nigral degeneration epilepsy, seizure disorders and other diseases related to general orexin system dysfunctions.

Compounds of the general formula (I) are particularly suitable for use in the treatment of diseases or disorders selected from the group consisting of eating disorders or sleep disorders.

Eating disorders may be defined as comprising metabolic dysfunction; dysregulated appetite control; compulsive obesities; emeto-bulimia or anorexia nervosa. This pathologically modified food intake may result from disturbed appetite (attraction or aversion for food); altered energy balance (intake vs expenditure); disturbed perception of food quality (high fat or carbohydrates, high palatability); disturbed food availability (unrestricted diet or deprivation) or disrupted water balance.

Sleep disorders include insomnias, narcolepsy and other disorders of excessive sleepiness, sleep-related dystonias; restless leg syndrome; sleep apneas; jet-lag syndrome; shift-work syndrome, delayed or advanced sleep phase syndrome. Insomnias are defined as comprising sleep disorders associated with aging; intermittent treatment of chronic insomnia; situational transient insomnia (new environment, noise) or short-term insomnia due to stress; grief; pain or illness.

The compounds of formula (I) and their pharmaceutically usable salts can be used as medicament (e.g. in the form of pharmaceutical preparations). The pharmaceutical preparations can be administered enterally, such as orally (e.g. in the form of tablets, coated tablets, dragées, hard and soft gelatine capsules, solutions, emulsions or suspensions), nasally (e.g. in the form of nasal sprays) or rectally (e.g. in the form of suppositories). However, the administration can also be effected parenterally, such as intramuscularly or intravenously (e.g. in the form of injection solutions), or topically, e.g. in the form of ointments, creams or oils.

The compounds of formula (I) and their pharmaceutically usable salts can be processed with pharmaceutically inert, inorganic or organic adjuvants for the production of tablets, coated tablets, dragées, and hard gelatine capsules. Lactose, cornstarch or derivatives thereof, talc, stearic acid or its salts etc. can be used, for example, as such adjuvants for tablets, dragées, and hard gelatine capsules. Suitable adjuvants for soft gelatine capsules, are, for example, vegetable oils, waxes, fats, semi-solid substances and liquid polyols, etc. Suitable adjuvants for the production of solutions and syrups are, for example, water, polyols, saccharose, invert sugar, glucose, etc. Suitable adjuvants for injection solutions are, for example, water, alcohols, polyols, glycerol, vegetable oils, etc. Suitable adjuvants for suppositories are, for example, natural or hardened oils, waxes, fats, semi-solid or liquid polyols, etc.

Moreover, the pharmaceutical preparations can contain preservatives, solubilizers, viscosity-increasing substances, stabilisers, wetting agents, emulsifiers, sweeteners, colorants, flavorants, salts for varying the osmotic pressure, buffers, masking agents or antioxidants. The compounds of formula (I) may also be used in combination with one or more other therapeutically useful substances. Examples are anorectic drugs like fenfluramine and related substances; lipase inhibitors like orlistat and related substances; antidepressants like fluoxetine and related substances; anxiolytics like alprazolam and related substances; sleep-inducers like zopiclone and related substances; or any other therapeutically useful substance.

The dosage of compounds of formula (I) can vary within wide limits depending on the disease to be controlled, the age and the individual condition of the patient and the mode of administration, and will, of course, be fitted to the individual requirements in each particular case. For adult patients a daily dosage of about 1 mg to 1000 mg, especially about 50 mg to about 500 mg, comes into consideration.

The pharmaceutical preparations conveniently contain about 1-500 mg, preferably 5-200 mg of a compound of formula (I).

The compounds of formula (I) of the present invention are prepared according to the general sequence of reactions outlined in the schemes below, wherein R¹, R², R³, R⁴, R⁵, R⁶ are as defined in formula (I) above. As the case may be any compound obtained with one or more optically active carbon atom may be resolved into pure enantiomers or diastereoisomers, mixtures of enantiomers or diastereoisomers, diastereoisomeric racemates and the meso-forms in a manner known per se.

The compounds obtained may also be converted into a pharmaceutically acceptable salt thereof in a manner known per se.

The compounds of formula (I) may be prepared as single compounds or as libraries of compounds comprising at least 2, typically 5 to 100 compounds of formula (I).

Compound libraries are prepared by multiple parallel synthesis using solution phase chemistry.

The compounds of formula (I) can be prepared as mixtures of all possible stereoisomers by following one of three possible synthetic pathways (Scheme 1). The first pathway starts with the reaction of the respective 1,2,3,4-tetrahydroisoquinoline with a methyl bromoacetate derivative. After saponification of the ester the obtained acid is coupled with ammonium bromide in the presence of EDC and DMAP to give the desired amides of formula (I).

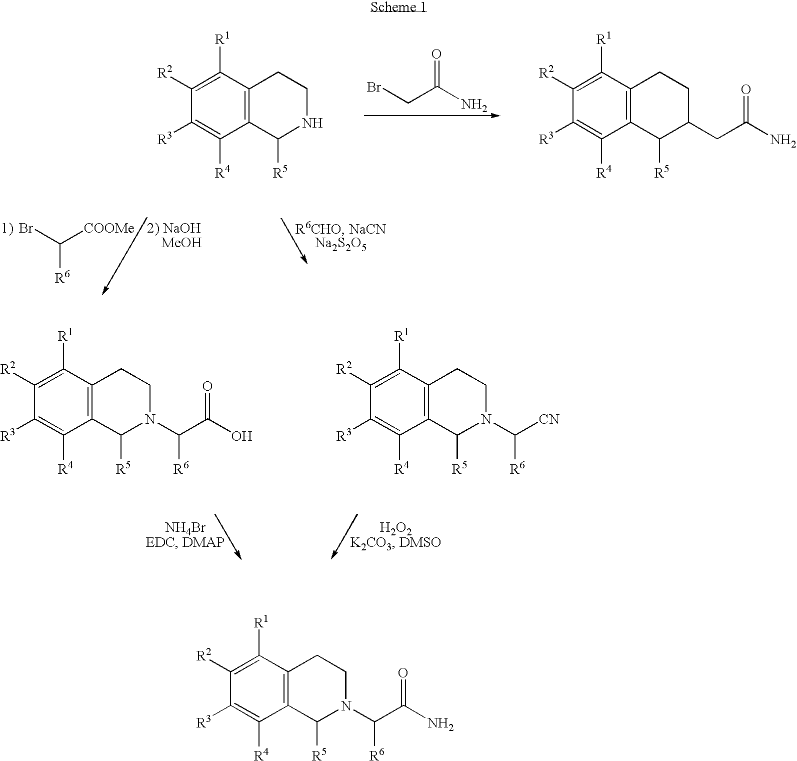

In a second pathway the 1,2,3,4-tetrahydroisoquinoline is reacted with an aldehyde and sodium cyanide to give the corresponding α-amino acetonitrile derivatives. These can be hydrolysed with hydrogen peroxide and potassium carbonate as base. In the case of the unsubstituted glycinamide analogues (R⁶=H) the final compounds of general formula (I) can be obtained by reaction of the 1,2,3,4-tetrahydroisoquinoline with commercially available 2-bromo-acetamide. The 1,2,3,4-tetrahydroisoquinolines, if not commercially available, can be prepared as racemic mixtures from the corresponding phenylethylamines by coupling with the desired carboxylic acid followed by treatment with POCl₃ and finally NaBH (see experimental part).

The stereoselective synthesis of the desired 1,2,3,4-tetrahydroisoquinoline derivatives is possible following one of the synthetic pathways shown in scheme 2. The key-intermediates, the 1-arylethyl-substituted 3,4-dihydroisoquinolines (R¹¹=aryl), can be obtained either by cyclisation of N-arylethyl-propionamides with POCl₃ or by alkylation of 1-methyl-3,4-dihydroisoquinoline derivatives with arylmethyl bromides or chlorides. These intermediates can be reduced to enantiomerically enriched 1,2,3,4-tetrahydro-isoquinoline derivatives by transfer hydrogenation in the presence of a chiral Ru(II) complex (chiral catalyst), which was originally described by R. Noyori et al. (J. Am. Chem. Soc. 1996, 118, 4916-4917 and WO 97/20789).

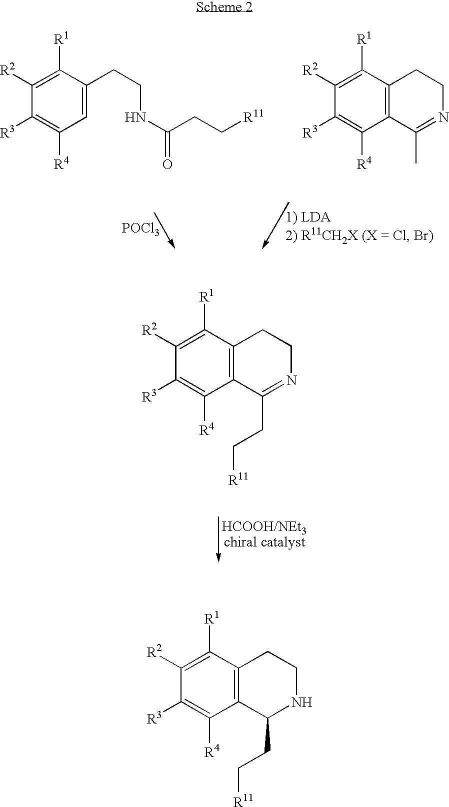

The chiral catalyst (Ru(II) complex) used is as follows:

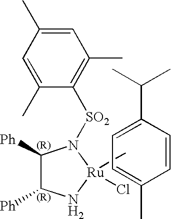

As illustrated in scheme 3 enantiomerically enriched 1,2,3,4-tetrahydroisoquinoline intermediates can be converted to compounds of formula (I) by alkylation with tosylated α-hydroxy acetamides. With racemic tosylates mixtures of diastereoisomers of formula (I) are obtained. Optically pure tosylates lead to essentially only one diastereoisomer. These can be prepared in a two step procedure starting from a single enantiomer of a 2-substituted methyl glycolate derivative by amidation and tosylation.

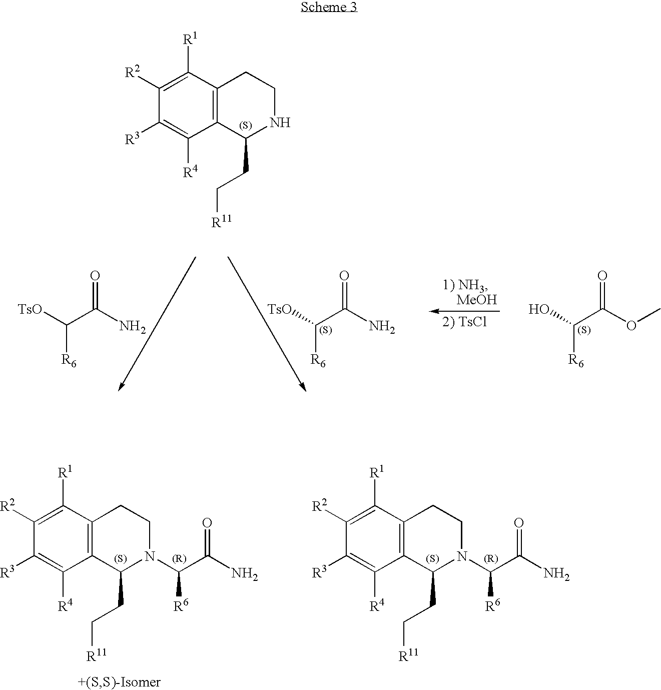

The 1,2,3,4-tetrahydroisoquinoline derivatives exemplified in this invention may be prepared from readily available starting materials using the following general methods and procedures. It will be appreciated that where typical or preferred experimental conditions (i.e., reaction temperatures, time, moles of reagents, solvents, etc.) are given, other experimental conditions can also be used unless otherwise stated. Optimum reaction conditions may vary with the particular reactants or solvents used, but such conditions can be determined by one skilled in the art using routine optimisation procedures.

Experimental Section

### Abbreviations

BSA Bovine serum albumine

CHO Chinese hamster ovary

d day(s)

dia diastereoisomer

DCM Dichloromethane

DIPEA Diisopropylethylamine

DMAP Dimethylaminopyridine

DMF N,N-dimethylformamide

DMSO Dimethylsulfoxide

EDC 1-(3-Dimethylaminopropyl)-3-ethylcarbodiimide

ES Electron spray

FCS Foetal calf serum

FLIPR Fluorescent imaging plate reader

h Hour(s)

HBSS Hank's balanced salt solution

HEPES 4-(2-Hydroxyethyl)-piperazine-1-ethanesulfonic acid

HPLC High pressure/performance liquid chromatography

MS Mass spectroscopy

LC Liquid chromatography

LDA Lithium diisopropylamide

min Minute(s)

prep preparative

rt retention time

RT Room temperature

sat saturated

THF Tetrahydrofuran

I. Biology

Determination of Orexin Receptor Antagonistic Activity

The Orexin receptor antagonistic activity of the compounds of formula (I) is determined in accordance with the following experimental method.

Experimental Method:

Intracellular Calcium Measurements

Chinese hamster ovary (CHO) cells expressing the human orexin-1 receptor and the human orexin-2 receptor, respectively, are grown in culture medium (Ham F-12 with L-Glutamine) containing 300 μg/ml G418, 100 U/ml penicillin, 100 μg/ml streptomycin and 10% inactivated foetal calf serum (FCS).

The cells are seeded at 80'000 cells/well into 96-well black clear bottom sterile plates (Costar) which have been precoated with 1% gelatine in Hanks' Balanced Salt Solution (HBSS). All reagents are from Gibco BRL.

The seeded plates were incubated overnight at 37° C. in 5% CO₂.

Human orexin-A as an agonist is prepared as 1 mM stock solution in methanol:water (1:1), diluted in HBSS containing 0.1% bovine serum albumin (BSA) and 2 mM HEPES for use in the assay at a final concentration of 10 nM.

Antagonists are prepared as 10 mM stock solution in DMSO, then diluted in 96-well plates, first in DMSO, then in HBSS containing 0.1% bovine serum albumin (BSA) and 2 mM HEPES.

On the day of the assay, 100 μl of loading medium (HBSS containing 1% FCS, 2 mM HEPES, 5 mM probenecid (Sigma) and 3 μM of the fluorescent calcium indicator fluo-3 AM (1 mM stock solution in DMSO with 10% pluronic acid) (Molecular Probes) is added to each well.

The 96-well plates are incubated for 60 min at 37° C. in 5% CO₂. The loading solution is then aspirated and cells are washed 3 times with 200 μl HBSS containing 2.5 mM probenecid, 0.1% BSA, 2 mM HEPES. 100 μl of that same buffer is left in each well.

Within the Fluorescent Imaging Plate Reader (FLIPR, Molecular Devices), antagonists are added to the plate in a volume of 50 μl, incubated for 20 min and finally 100 μl of agonist is added. Fluorescence is measured for each well at 1 second intervals, and the height of each fluorescence peak is compared to the height of the fluorescence peak induced by 10 nM orexin-A with buffer in place of antagonist. For each antagonist, IC₅₀ value (the concentration of compound needed to inhibit 50% of the agonistic response) is determined. The compounds exhibit as an average an antagonistic activity regarding the OX₁ and OX₂ receptor in the range of an IC₅₀ of 1 to 100 nM.

II. Chemistry

The following examples illustrate the preparation of pharmacologically active compounds of the invention but do not at all limit the scope thereof.

All temperatures are stated in ° C.

All analytical and preparative HPLC investigations on non-chiral phases are performed using RP-C18 based columns. Analytical HPLC investigations are performed on two different instruments with cycle-times of ˜2.5 min and ˜13 min respectively. For HPLC separations on chiral phases a Chiralcel OD column from Daicel Chemical Industries is used.

A Synthesis of Phenylethylamide Derivatives:

Procedure 1:

A solution of the respective phenylethylamine (82.8 mmol) and the respective carboxylic acid (82.8 mmol) in toluene (330 mL) is treated with 2 crystals of p-toluenesulfonic acid and refluxed for 14 h in the presence of a Dean-Stark. The solvent is removed in vacuo and the residue is recrystalized from toluene to give the following amides:

### N-[2-(3,4-Dimethoxy-phenyl)-ethyl]-2-naphthalen-2-yl-acetamide

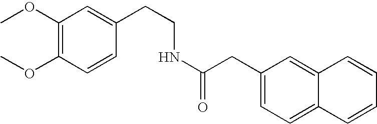

prepared by reaction of 3,4-dimethoxyphenylethylamine and 2-naphthylacetic acid.

### 3-(3,4-Dimethoxy-phenyl)-N-[2-(3,4-dimethoxy-phenyl)-ethyl]-propionamide

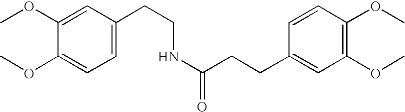

prepared by reaction of 3-(3,4-dimethoxyphenyl)-propionic acid and 3,4-dimethoxy-phenylethylamine.

LC-MS: rt=0.89 min, 374 (M+1, ES+).

### N-[2-(3,4-Dimethoxy-phenyl)-ethyl]-3-(4-trifluoromethyl-phenyl)-propionamide

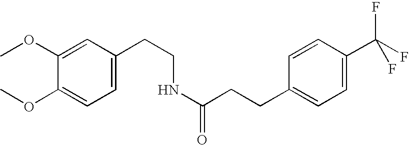

prepared by reaction of 3-(4-trifluoromethylphenyl)-propionic acid and 3,4-dimethoxy-phenylethylamine.

LC-MS: rt=1.03 min, 382 (M+1, ES+).

### N-[2-(3,4-Dimethoxy-phenyl)-ethyl]-2-(4-fluoro-phenyl)-acetamide

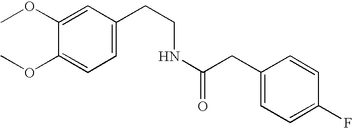

prepared by reaction of 2-(4-fluorophenyl)-acetic acid and 3,4-dimethoxy-phenylethylamine.

LC-MS: rt=4.11 min, 318 (M+1, ES+).

Procedure 2:

A solution of the respective carboxylic acid (11.0 mmol) in THF (20 mL) is treated with EDC hydrochloride (11.0 mmol). After 5 min the respective amine (11.0 mmol) is added and the reaction mixture is stirred for 14 h. Ethyl acetate is added and the organic layer is washed with sat. aqueous NaHCO₃ solution, 10% aqueous citric acid solution and brine. The organic layer is dried with MgSO₄ and filtered. After evaporation of the solvents the following crude amides are obtained which are used in the following step without further purification:

### 3-(3,4-Difluoro-phenyl)-N-[2-(3,4-dimethoxy-phenyl)-ethyl]-propionamide

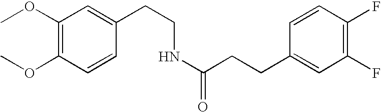

prepared by reaction of 3-(3,4-difluorophenyl)-propionic acid and 3,4-dimethoxy-phenylethylamine.

### 3-[2,5-Bis(trifluoromethyl)-phenyl]-N-[2-(3,4-dimethoxy-phenyl)-ethyl]-propionamide

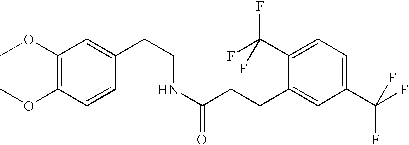

prepared by reaction of 3-[2,5-bis(trifluoromethyl)-phenyl]-propionic acid and 3,4-dimethoxy-phenylethylamine.

### 3-(2,5-Difluoro-phenyl)-N-[2-(3,4-dimethoxy-phenyl)-ethyl]-propionamide

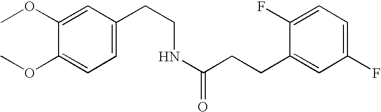

prepared by reaction of 3-(2,5-difluorophenyl)-propionic acid and 3,4-dimethoxy-phenylethylamine.

### N-[2-(3,4-Dimethoxy-phenyl)-ethyl]-3-(2-methoxy-phenyl)-propionamide

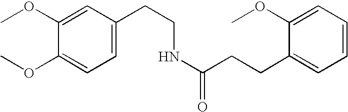

prepared by reaction of 3-(2-methoxyphenyl)-propionic acid and 3,4-dimethoxy-phenylethylamine.

### N-[2-(3,4-Dimethoxy-phenyl)-ethyl]-3-(4-fluoro-phenyl)-propionamide

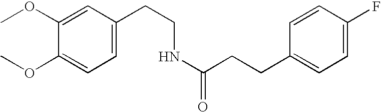

prepared by reaction of 3-(4-fluorophenyl)-propionic acid and 3,4-dimethoxy-phenylethylamine.

### 3-(2,3-Difluoro-phenyl)-N-[2-(3,4-dimethoxy-phenyl)-ethyl]-acrylamide

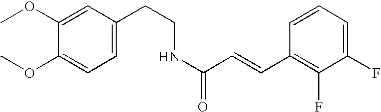

prepared by reaction of 3-(2,3-difluoro-phenyl)-acrylic acid and 3,4-dimethoxy-phenylethylamine.

### N-[2-(3,4-Dimethoxy-phenyl)-ethyl]-2-(3-fluoro-4-methoxy-phenyl)-acetamide

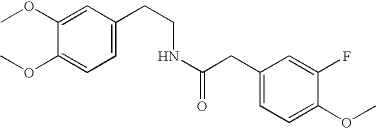

prepared by reaction of 2-(3-fluoro-4-methoxy-phenyl)-acetic acid and 3,4-dimethoxy-phenylethylamine.

### 3-(2,3-Difluoro-phenyl)-N-[2-(3,4-dimethoxy-phenyl)-ethyl]-propionamide

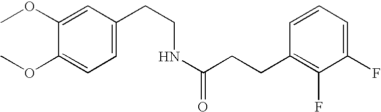

prepared by reaction of 3-(2,3-difluorophenyl)-propionic acid and 3,4-dimethoxy-phenylethylamine.

### N-[2-(3,4-Dimethoxy-phenyl)-ethyl]-3-(3-methoxy-phenyl)-propionamide

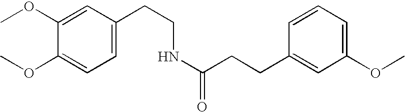

prepared by reaction of 3-(3-methoxyphenyl)-propionic acid and 3,4-dimethoxy-phenylethylamine.

### 3-(2,5-Difluoro-phenyl)-N-[2-(3,4-dimethoxy-phenyl)-ethyl]-acrylamide

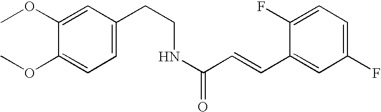

prepared by reaction of 3-(2,5-difluoro-phenyl)-acrylic acid and 3,4-dimethoxy-phenylethylamine.

Procedure 3:

To a solution of the respective carboxylic acid (21.4 mmol) in DMF (110 mL) is added successively PyBOP (23.6 mmol), 3,4-dimethoxy-phenylethylamine (21.4 mmol) and N-diisopropylethylamine (49.3 mmol). After stirring for 8 h at RT ethyl acetate (100 mL) is added and the organic layer is washed three times with brine (3×70 mL). The organic layer is dried with MgSO₄ and filtered. The solvent is removed in vacuo und the residue is purified by flash-chromatography (DCM/MeOH 36/1) to give the following amides:

### N-[2-(3,4-Dimethoxy-phenyl)-ethyl]-3-furan-2-yl-propionamide

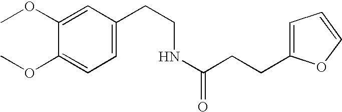

prepared by reaction of 3-furan-2-yl-propionic acid and 3,4-dimethoxy-phenylethylamine.

LC-MS: rt=3.96 min, 304 (M+1, ES+).

### N-[2-(3,4-Dimethoxy-phenyl)-ethyl]-4-phenyl-butyramide

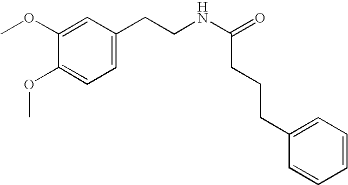

prepared by reaction of 4-phenyl-butyric acid and 3,4-dimethoxy-phenylethylamine.

LC-MS: rt=4.47 min, 328 (M+1, ES+).

Procedure 4:

A solution of the respective phenylethylamine (80 mmol) and of triethylamine (90 mmol) in THF (120 mL) is cooled to 0° C. and treated portionwise with the respective carboxylic acid chloride (80 mmol). After stirring for 10 min at 0° C. and for 14 h at RT a sat. aqueous NaHCO₃ solution is added, the layers are separated and the aqueous layer is extracted three times with ethyl acetate (150 mL). The solvent is removed in vacuo and the residue is either recrystallized from toluene or purified by flash chromatography to give the following amides:

### 2-(2,5-Dimethoxy-phenyl)-N-[2-(2,5-dimethoxy-phenyl)ethyl]-acetamide

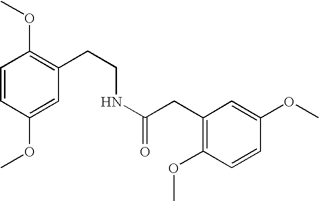

prepared by reaction of (2,5-dimethoxy-phenyl)-acetyl chloride and 2,5-dimethoxy-phenylethylamine.

LC-MS: rt=4.62 min, 360 (M+1, ES+).

### N-[2-(3-Methoxy-phenyl)-ethyl]-2-phenyl-acetamide

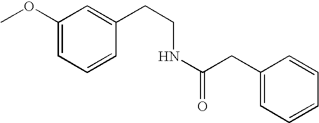

prepared by reaction of phenyl-acetyl chloride and 3-methoxy-phenylethylamine.

LC-MS: rt=4.36 min, 270 (M+1, ES+).

### 2-(2,5-Dimethoxy-phenyl)-N-[2-(3,4-dimethoxy-phenyl)-ethyl]-acetamide

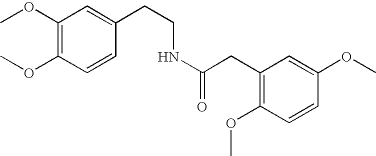

prepared by reaction of (2,5-dimethoxy-phenyl)-acetyl chloride and 3,4-dimethoxy-phenylethylamine.

LC-MS: rt=4.11 min, 360 (M+1, ES+).

### N-[2-(3,4-Dimethoxy-phenyl)-ethyl]-2-(4-methoxy-phenyl)-acetamide

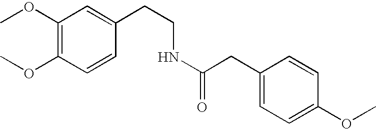

prepared by reaction of (4-methoxy-phenyl)-acetyl chloride and 3,4-dimethoxy-phenylethylamine.

LC-MS: rt=3.99 min, 330 (M+1, ES+).

### N-[2-(3,4-Dimethoxy-phenyl)-ethyl]-2-(3-methoxy-phenyl)-acetamide

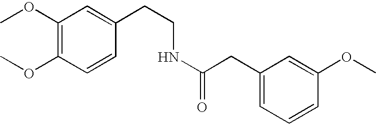

prepared by reaction of (3-methoxy-phenyl)-acetyl chloride and 3,4-dimethoxy-phenylethylamine.

LC-MS: rt=4.03 min, 330 (M+1, ES+).

### N-[2-(2,3-Dihydro-benzo[1,4]dioxin-6-yl)-ethyl]-2-(3,4-dimethoxy-phenyl)-acetamide

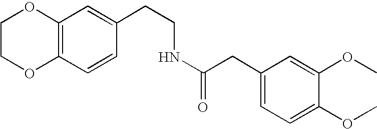

prepared by reaction of (3,4-dimethoxy-phenyl)-acetyl chloride and 2-(2,3-dihydro-benzo[1,4]dioxin-6-yl)-ethylamine.

LC-MS: rt=4.03 min, 358 (M+1, ES+).

### N-[2-(3,4-Dimethoxy-phenyl)-ethyl]-2-phenoxy-acetamide

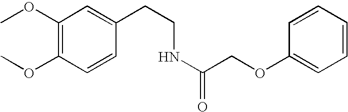

prepared by reaction of phenoxy-acetyl chloride and 3,4-dimethoxy-phenyl-ethylamine.

LC-MS: rt=4.24 min, 316 (M+1, ES+).

B Synthesis of 3,4-Dihydroisoquinoline Derivatives (General Procedure):

Phosphorus oxychloride (123 mmol) is added to a suspension of the respective amide (55.3 mmol) in acetonitrile (300 mL). The mixture is refluxed for 90 min and the solvents are removed in vacuo. Methanol (100 mL) is added and evaporated again. The obtained product is recrystallized from dioxane or dioxane/ethanol. After filtration the obtained hydrochloride salt is converted to the free base by addition of saturated aqueous NaHCO₃ solution and extraction with dichloromethane. The solvents are removed in vacuo to give the respective 3,4-di-hydroisoquinoline.

### 6,7-dimethoxy-1-[2-(4-trifluoromethyl-phenyl)-ethyl]-3,4-dihydroisoquinoline

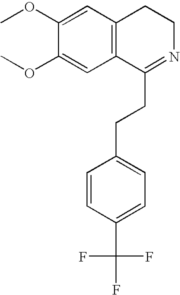

prepared by cyclisation of N-[2-(3,4-dimethoxy-phenyl)-ethyl]-3-(4-trifluoromethyl-phenyl)-propionamide.

LC-MS: rt=0.81 min, 364 (M+1, ES+).

C Synthesis of 1,2,3,4-Tetrahydroisoquinoline Derivatives:

C.1 Synthesis of 1,2,3,4-Tetrahydroisoquinolines via Bischler-Napieralski-Reaction (General Procedure):

To a suspension of the respective acetamide (6.0 mmol) in acetonitrile (80 mL) is added phosphorus oxychloride (2.8 mL, 30 mmol). The mixture is heated to reflux for 2 h and the solvent is removed in vacuo. The resulting oil is taken up in MeOH (10 mL), evaporated to dryness, dissolved in MeOH (35 mL) and cooled to 0° C. NaBH₄ (30 mmol) is added in small (!) portions and the reaction mixture is stirred for 14 h. The solvent is removed in vacuo, ethyl acetate (100 mL) and water (150 mL) are added, the layers are separated and the aqueous layer is extracted three times with ethyl acetate (50 mL). The combined organic extracts are concentrated in vacuo to give the following tetrahydroisoquinolines as racemic mixtures, which are purified by crystallization as hydrochloride salt:

### 6,7-Dimethoxy-1-naphthalen-2-ylmethyl-1,2,3,4-tetrahydroisoquinoline

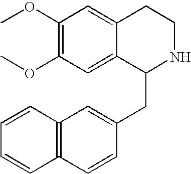

prepared by cyclisation of N-[2-(3,4-dimethoxy-phenyl)-ethyl]-2-naphthalen-2-yl-acetamide.

LC-MS: rt=0.75 min, 334 (M+1, ES+), 375 (M+CH₃CN, ES+).

### 1-[2-(3,4-Difluoro-phenyl)-ethyl]-6,7-dimethoxy-1,2,3,4-tetrahydroisoquinoline

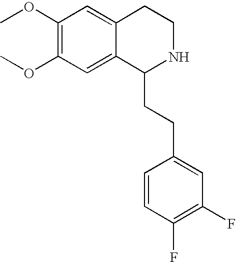

prepared by cyclisation of 3-(3,4-difluoro-phenyl)-N-[2-(3,4-dimethoxy-phenyl)-ethyl]-propionamide.

LC-MS: rt=0.82 min, 334 (M+1, ES+), 375 (M+CH₃CN, ES+).

### 1-[2-(2,5-Bis-trifluoromethyl-phenyl)-ethyl]-6,7-dimethoxy-1,2,3,4-tetrahydroisoquinoline

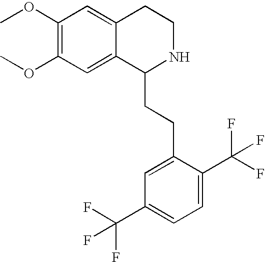

prepared by cyclisation of 3-[2,5-bis(trifluoromethyl)-phenyl]-N-[2-(3,4-dimethoxy-phenyl)-ethyl]-propionamide.

LC-MS: rt=0.89 min, 434 (M+1, ES+), 475 (M+CH₃CN, ES+).

### 1-[2-(2,5-Difluoro-phenyl)-ethyl]-6,7-dimethoxy-1,2,3,4-tetrahydroisoquinoline

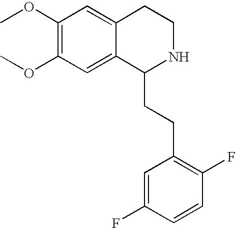

prepared by cyclisation of 3-(2,5-difluoro-phenyl)-N-[2-(3,4-dimethoxy-phenyl)-ethyl]-propionamide.

LC-MS: rt=0.80 min, 334 (M+1, ES+).

### 6,7-Dimethoxy-1-[2-(2-methoxy-phenyl)-ethyl]-1,2,3,4-tetrahydroisoquinoline

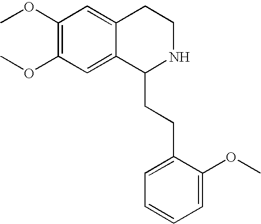

prepared by cyclisation of N-[2-(3,4-dimethoxy-phenyl)-ethyl]-3-(2-methoxy-phenyl)-propionamide.

LC-MS: rt=0.81 min, 328 (M+1, ES+).

### 1-[2-(4-Fluoro-phenyl)-ethyl]-6,7-dimethoxy-1,2,3,4-tetrahydroisoquinoline

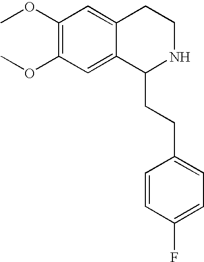

prepared by cyclisation of N-[2-(3,4-dimethoxy-phenyl)-ethyl]-3-(4-fluoro-phenyl)-propionamide.

LC-MS: rt=0.81 min, 316 (M+1, ES+).

### 1-[2-(2,3-Difluoro-phenyl)-vinyl]-6,7-dimethoxy-1,2,3,4-tetrahydroisoquinoline

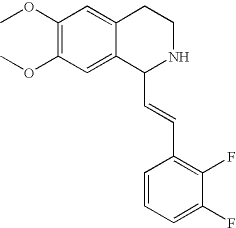

prepared by cyclisation of 3-(2,3-difluoro-phenyl)-N-[2-(3,4-dimethoxy-phenyl)-ethyl]-acrylamide.

LC-MS: rt=0.81 min, 332 (M+1, ES+).

### 1-(3-Fluoro-4-methoxy-benzyl)-6,7-dimethoxy-1,2,3,4-tetrahydroisoquinoline

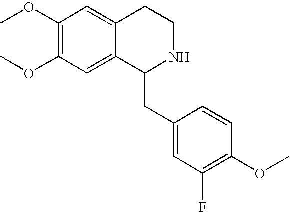

prepared by cyclisation of N-[2-(3,4-dimethoxy-phenyl)-ethyl]-2-(3-fluoro-4-methoxy-phenyl)-acetamide.

LC-MS: rt=0.78 min, 332 (+1, ES+).

### 1-[2-(3,4-Dimethoxy-phenyl)-ethyl]-6,7-dimethoxy-1,2,3,4-tetrahydroisoquinoline

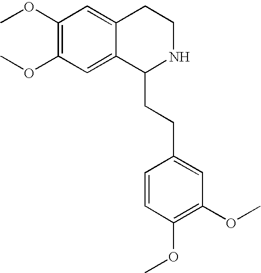

prepared by cyclisation of 3-(3,4-dimethoxy-phenyl)-N-[2-(3,4-dimethoxy-phenyl)-ethyl]-propionamide.

LC-MS: rt=0.76 min, 358 (M+1, ES+).

### 1-[2-(2,3-Difluoro-phenyl)-ethyl]-6,7-dimethoxy-1,2,3,4-tetrahydroisoquinoline

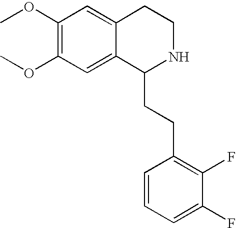

prepared by cyclisation of 3-(2,3-difluoro-phenyl)-N-[2-(3,4-dimethoxy-phenyl)-ethyl]-propionamide.

LC-MS: rt=0.80 min, 334 (M+1, ES+).

### 6,7-Dimethoxy-1-[2-(4-trifluoromethyl-phenyl)-ethyl]-1,2,3,4-tetrahydroisoquinoline

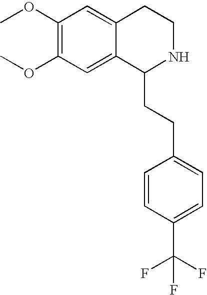

prepared by cyclisation of N-[2-(3,4-dimethoxy-phenyl)-ethyl]-3-(4-trifluoromethyl-phenyl)-propionamide.

LC-MS: rt=0.85 min, 366 (M+1, ES+).

### 6,7-Dimethoxy-1-[2-(3-methoxy-phenyl)-ethyl]-1,2,3,4-tetrahydroisoquinoline

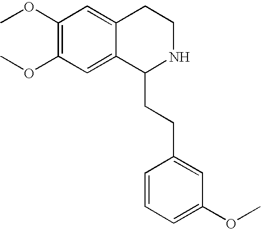

prepared by cyclisation of N-[2-(3,4-dimethoxy-phenyl)-ethyl]-3-(3-methoxyphenyl)-propionamide.

LC-MS: rt=0.79 min, 328 (M+1, ES+).

### 1-[2-(2,5-Difluoro-phenyl)-vinyl]-6,7-dimethoxy-1,2,3,4-tetrahydroisoquinoline

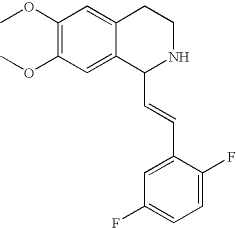

prepared by cyclisation of 3-(2,5-difluoro-phenyl)-N-[2-(3,4-dimethoxy-phenyl)-ethyl]-acrylamide.

LC-MS: rt=0.80 min, 332 (M+1, ES+).

### 1-(2-Furan-2-yl-ethyl)-6,7-dimethoxy-1,2,3,4-tetrahydroisoquinoline

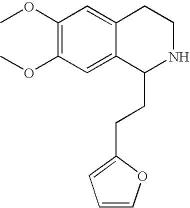

prepared by cyclisation of N-[2-(3,4-dimethoxy-phenyl)-ethyl]-3-furan-2-yl-propionamide.

LC-MS: rt=2.70 min, 288 (M+1, ES+).

### 1-(2,5-Dimethoxy-benzyl)-5,8-dimethoxy-1,2,3,4-tetrahydroisoquinoline

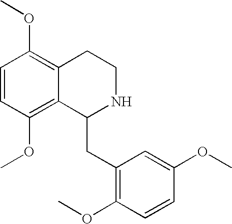

prepared by cyclisation of 2-(2,5-dimethoxy-phenyl)-N-[2-(2,5-dimethoxy-phenyl)-ethyl]-acetamide.

LC-MS: rt=3.57 min, 344 (M+1, ES+).

### 1-Benzyl-6-methoxy-1,2,3,4-tetrahydro-isoquinoline

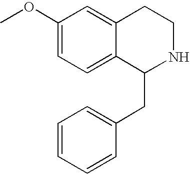

prepared by cyclisation of N-[2-(3-methoxy-phenyl)-ethyl]-2-phenyl-acetamide.

LC-MS: rt=3.12 min, 254 (M+1, ES+).

### 1-(4-Fluoro-benzyl)-6,7-dimethoxy-1,2,3,4-tetrahydro-isoquinoline

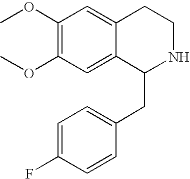

prepared by cyclisation of N-[2-(3,4-dimethoxy-phenyl)-ethyl]-2-(4-fluoro-phenyl)-acetamide.

LC-MS: rt=3.05 min, 302 (M+1, ES+).

### 1-(2,5-Dimethoxy-benzyl)-6,7-dimethoxy-1,2,3,4-tetrahydro-isoquinoline

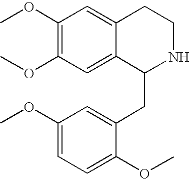

prepared by cyclisation of 2-(2,5-dimethoxy-phenyl)-N-[2-(3,4-dimethoxy-phenyl)-ethyl]-acetamide.

LC-MS: rt=3.18 min, 344 (M+1, ES+).

### 6,7-Dimethoxy-1-(4-methoxy-benzyl)-1,2,3,4-tetrahydro-isoquinoline

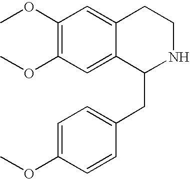

prepared by cyclisation of N-[2-(3,4-dimethoxy-phenyl)-ethyl]-2-(4-methoxy-phenyl)-acetamide.

LC-MS: rt=3.01 min, 314 (M+1, ES+).

### 6,7-Dimethoxy-1-phenoxymethyl-1,2,3,4-tetrahydro-isoquinoline

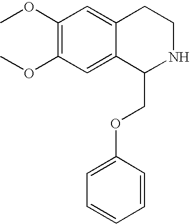

prepared by cyclisation of N-[2-(3,4-dimethoxy-phenyl)-ethyl]-2-phenoxy-acetamide.

LC-MS: rt 3.02 min, 300 (M+1, ES+).

### 6,7-Dimethoxy-1-(3-methoxy-benzyl)-1,2,3,4-tetrahydro-isoquinoline

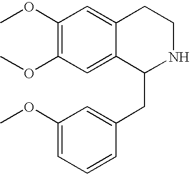

prepared by cyclisation of N-[2-(3,4-dimethoxy-phenyl)-ethyl]-2-(3-methoxy-phenyl)-acetamide.

LC-MS: rt=3.04 min, 314 (M+1, ES+).

### 6-(3,4-Dimethoxy-benzyl)-2,3,6,7,8,9-hexahydro-[1,4]dioxino[2,3-g]isoquinoline

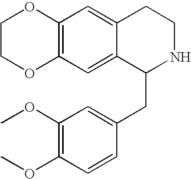

prepared by cyclisation of N-[2-(2,3-dihydro-benzo[1,4]dioxin-6-yl)-ethyl]-2-(3,4-dimethoxy-phenyl)-acetamide.

LC-MS: rt=3.07 min, 342 (M+1, ES+).

### 6,7-Dimethoxy-1-(3-phenyl-propyl)-1,2,3,4-tetrahydro-isoquinoline

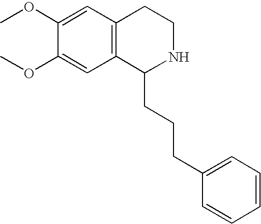

prepared by cyclisation of N-[2-(3,4-dimethoxy-phenyl)-ethyl]-4-phenyl-butyramide.

LC-MS: rt=3.15 min, 312 (M+1, ES+).

C.2 Stereoselective Synthesis of 1,2,3,4-Tetrahydroisoquinoline Derivatives via Transfer Hydrogenation (General Procedure):

Dichloro-(p-cymene)ruthenium (II) dimer (0.20 mmol) is added to a solution of N-((1R,2R)-2-amino-1,2-diphenyl-ethyl)-2,4,6-trimethylbenzene-sulfonamide (0.40 mmol) and triethylamine (0.80 mmol) in acetonitrile (3.0 mL). The mixture is stirred for 1 h at 80° C. and added to a solution of the respective dihydroisoquinoline (28.0 mmol) in dichloromethane (30 mL). An azeotropic mixture of formic acid and triethylamine (5:2, 14 mL) is added (gas evolution). After 90 min a sat. aqueous NaHCO₃ solution (200 mL) is added to the dark red solution. The layers are separated, the aqueous layer is extracted twice with DCM (2×200 mL) and the combined organic extracts are concentrated in vacuo. The residue is dissolved in isopropanol (1600 mL) and treated with a solution of HCl in isopropanol (5-6 M, 10 mL). The obtained hydrochloride salt is recrystallized to give the respective 1,2,3,4-tetrahydroisoquinoline with high enantiomeric excess as determined by chiral HPLC. The hydrochloride salt is converted to the free base by extraction with sat. NaHCO₃ solution/dichloromethane. The absolute configuration of the respective product is assigned in analogy to the literature (N. Uematsu, A. Fujii, S. Hashiguchi, T. Ikariya, R. Noyori, J. Am. Chem. Soc. 1996, 118, 4916-4917).

### (S)-6,7-Dimethoxy-1-[2-(4-trifluoromethyl-phenyl)-ethyl]-1,2,3,4-tetrahydroisoquinoline

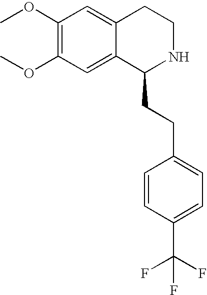

prepared by transfer hydrogenation of 6,7-dimethoxy-1-[2-(4-trifluoromethyl-phenyl)-ethyl]-3,4-dihydroisoquinoline.

LC-MS: rt=0.80 min, 366 (M+1, ES+).

chiral HPLC: rt=12.0 min (hexane/ethanol 9/1; enantiomer: rt=17.1 min).

C.3 Stereoselective Synthesis of 1,2,3,4-Tetrahydroisoquinoline Derivatives via Alkylation of 1-Methyl-3,4-dihydroisoquinolines (General Procedure):

At 0° C. a solution of n-BuLi in hexane (1.6M, 0.63 mmol) is added drop wise to a mixture of 6,7-dimethoxy-1-methyl-3,4-dihydroisoquinoline (0.50 mmol) and di-isopropylamine (0.63 mmol) in THF (1.0 mL). The reaction mixture is stirred at RT for 1 h and added at 0° C. to a solution of the respective benzyl bromide (0.50 mmol) in THF (1.0 mL). The solution is stirred for 1 h, warmed up to RT and diluted with DCM (3.0 mL).

In a second flask dichloro(p-cymene)ruthenium (II) dimer (0.15 mmol) is added to a solution of N-((1R,2R)-2-amino-1,2-diphenyl-ethyl)-2,4,6-trimethyl-benzene-sulfonamide (0.30 mmol) and triethylamine (0.60 mmol) in acetonitrile (3.3 mL). The mixture is stirred for 1 h at 80° C. A portion of this solution (0.10 mL) is added to the solution of the respective dihydroisoquinoline (described above). An azeotropic mixture of formic acid and triethylamine (5:2, 0.3 mL) is added (gas evolution). After 2d the mixture is concentrated in vacuo and purified by prep. HPLC to give the respective 1,2,3,4-tetrahydroisoquinoline.

The absolute configuration of the respective product is assigned in analogy to the literature (N. Uematsu, A. Fujii, S. Hashiguchi, T. Ikariya, R. Noyori, J. Am. Chem. Soc 1996, 118, 4916-4917).

### (S)-6,7-Dimethoxy-1-[2-(2-methyl-5-trifluoromethyl-phenyl)-ethyl]-1,2,3,4-tetrahydro-isoquinoline

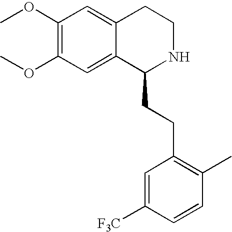

prepared from 6,7-dimethoxy-1-methyl-3,4-dihydro-isoquinoline and 2-chloro-methyl-1-methyl-4-trifluoromethyl-benzene.

LC-MS: rt=0.84 min, 380 (M+1, ES+).

### (S)-1-[2-(3-Chloro-2-fluoro-phenyl)-ethyl]-6,7-dimethoxy-1,2,3,4-tetrahydroisoquinoline

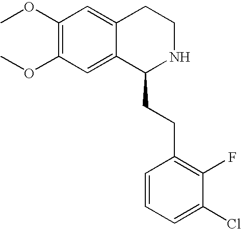

prepared from 6,7-dimethoxy-1-methyl-3,4-dihydro-isoquinoline and 1-bromo-methyl-3-chloro-2-fluoro-benzene.

LC-MS: rt=0.80 min, 350 (M+1, ES+).

### (S)-1-[2-(3-Fluoro-4-methyl-phenyl)-ethyl]-6,7-dimethoxy-1,2,3,4-tetrahydroisoquinoline

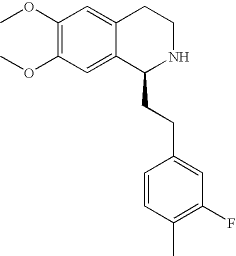

prepared from 6,7-dimethoxy-1-methyl-3,4-dihydro-isoquinoline and 4-bromo-methyl-2-fluoro-1-methyl-benzene.

LC-MS: rt=0.80 min, 330 (M+1, ES+).

### (S)-1-[2-(4-Fluoro-2-trifluoromethyl-phenyl)-ethyl]-6,7-dimethoxy-1,2,3,4-tetrahydro-isoquinoline

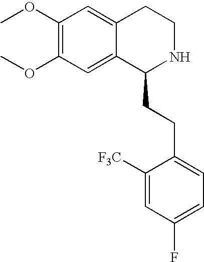

prepared from 6,7-dimethoxy-1-methyl-3,4-dihydro-isoquinoline and 1-bromo-methyl-4-fluoro-2-trifluoromethyl-benzene.

LC-MS: rt=0.82 min, 384 (M+1, ES+).

### (S)-1-[2-(5-Fluoro-2-trifluoromethyl-phenyl)-ethyl]-6,7-dimethoxy-1,2,3,4-tetrahydro-isoquinoline

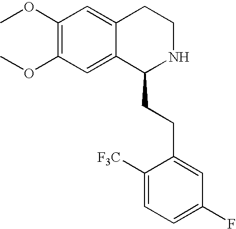

prepared from 6,7-dimethoxy-1-methyl-3,4-dihydro-isoquinoline and 2-bromo-methyl-4-fluoro-1-trifluoromethyl-benzene.

LC-MS: rt=0.81 min, 384 (M+1, ES+).

### (S)-1-[2-(2-Fluoro-5-trifluoromethyl-phenyl)-ethyl]-6,7-dimethoxy-1,2,3,4-tetrahydro-isoquinoline

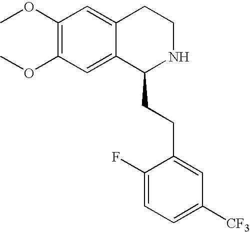

prepared from 6,7-dimethoxy-1-methyl-3,4-dihydro-isoquinoline and 2-bromo-methyl-1-fluoro-4-trifluoromethyl-benzene.

LC-MS: rt=0.82 min, 384 (M+1, ES+).

### (S)-1-[2-(3-Fluoro-4-trifluoromethyl-phenyl)-ethyl]-6,7-dimethoxy-1,2,3,4-tetrahydro-isoquinoline

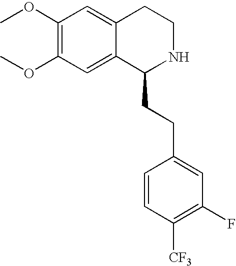

prepared from 6,7-dimethoxy-1-methyl-3,4-dihydro-isoquinoline and 4-bromo-methyl-2-fluoro-1-trifluoromethyl-benzene.

LC-MS: rt=0.83 min, 384 (M+1, ES+).

### (S)-1-[2-(4-Fluoro-3-methyl-phenyl)-ethyl]-6,7-dimethoxy-1,2,3,4-tetrahydro-isoquinoline

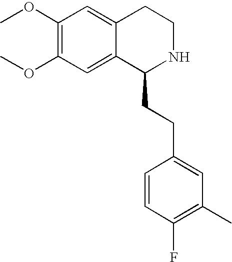

prepared from 6,7-dimethoxy-1-methyl-3,4-dihydro-isoquinoline and 4-bromo-methyl-1-fluoro-2-methyl-benzene.

LC-MS: rt=0.80 min, 330 (M+1, ES+).

### (S)-6,7-Dimethoxy-1-[2-(2,3,5-trifluoro-phenyl)-ethyl]-1,2,3,4-tetrahydro-isoquinoline

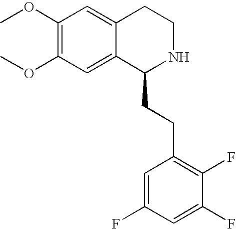

prepared from 6,7-dimethoxy-1-methyl-3,4-dihydro-isoquinoline and 1-bromo-methyl-2,3,5-trifluoro-benzene.

LC-MS: rt=0.78 min, 352 (M+1, ES+).

### (S)-1-[2-(4-Fluoro-3-trifluoromethyl-phenyl)-ethyl]-6,7-dimethoxy-1,2,3,4-tetrahydro-isoquinoline

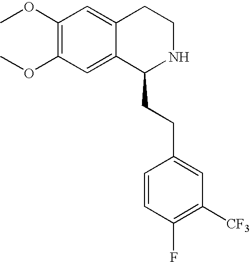

prepared from 6,7-dimethoxy-1-methyl-3,4-dihydro-isoquinoline and 4-bromo-methyl-1-fluoro-2-trifluoromethyl-benzene.

LC-MS: rt=0.83 min, 384 (M+1, ES+).

### (S)-1-[2-(3-Fluoro-5-trifluoromethyl-phenyl)-ethyl]-6,7-dimethoxy-1,2,3,4-tetrahydro-isoquinoline

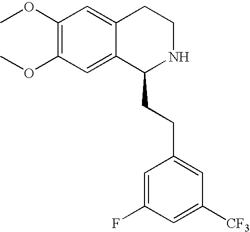

prepared from 6,7-dimethoxy-1-methyl-3,4-dihydro-isoquinoline and 1-bromo-methyl-3-fluoro-5-trifluoromethyl-benzene.

LC-MS: rt=0.83 min, 384 (M+1, ES+).

### (S)-1-[2-(5-Chloro-2-trifluoromethyl-phenyl)-ethyl]-6,7-dimethoxy-1,2,3,4-tetrahydro-isoquinoline

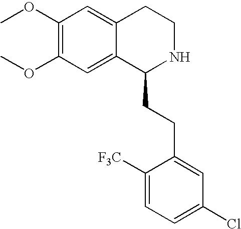

prepared from 6,7-dimethoxy-1-methyl-3,4-dihydro-isoquinoline and 2-bromo-methyl-4-chloro-1-trifluoromethyl-benzene.

LC-MS: rt=0.84 min, 400 (M+1, ES+).

### (S)-1-[2-(2-Fluoro-3-trifluoromethyl-phenyl)-ethyl]-6,7-dimethoxy-1,2,3,4-tetrahydro-isoquinoline

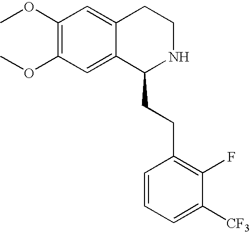

prepared from 6,7-dimethoxy-1-methyl-3,4-dihydro-isoquinoline and 1-bromo-methyl-2-fluoro-3-trifluoromethyl-benzene.

LC-MS: rt=0.82 min, 384 (M+1, ES+).

### (S)-1-[2-(2-Fluoro-4-trifluoromethyl-phenyl)-ethyl]-6,7-dimethoxy-1,2,3,4-tetrahydro-isoquinoline

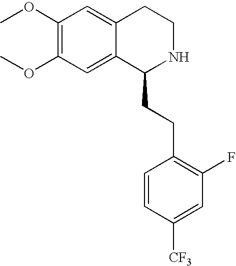

prepared from 6,7-dimethoxy-1-methyl-3,4-dihydro-isoquinoline and 1-bromo-methyl-2-fluoro-4-trifluoromethyl-benzene.

LC-MS: rt=0.83 min, 384 (M+1, ES+).

### (S)-1-[2-(2-Difluoromethoxy-phenyl)-ethyl]-6,7-dimethoxy-1,2,3,4-tetrahydro-isoquinoline

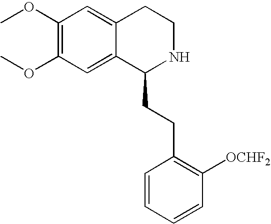

prepared from 6,7-dimethoxy-1-methyl-3,4-dihydro-isoquinoline and 1-bromo-methyl-2-difluoromethoxy-benzene.

LC-MS: rt=0.78 min, 364 (M+1, ES+).

### (S)-1-[2-(3-Fluoro-2-trifluoromethyl-phenyl)-ethyl]-6,7-dimethoxy-1,2,3,4-tetrahydro-isoquinoline

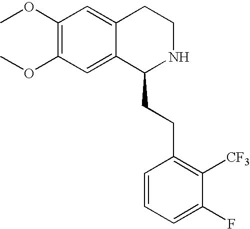

prepared from 6,7-dimethoxy-1-methyl-3,4-dihydro-isoquinoline and 1-bromo-methyl-3-fluoro-2-trifluoromethyl-benzene.

LC-MS: rt=0.81 min, 384 (M+1, ES+).

### (S)-1-[2-(2-Chloro-3-trifluoromethyl-phenyl)-ethyl]-6,7-dimethoxy-1,2,3,4-tetrahydro-isoquinoline

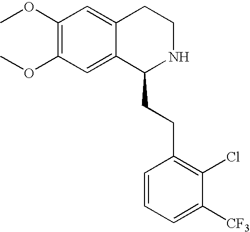

prepared from 6,7-dimethoxy-1-methyl-3,4-dihydro-isoquinoline and 1-bromo-methyl-2-chloro-3-trifluoromethyl-benzene.

LC-MS: rt=0.84 min, 400 (M+1, ES+).

### (S)-1-[2-(2-Fluoro-6-trifluoromethyl-phenyl)-ethyl]-6,7-dimethoxy-1,2,3,4-tetrahydro-isoquinoline

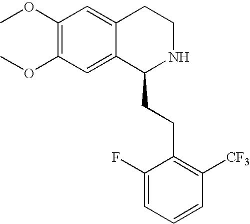

prepared from 6,7-dimethoxy-1-methyl-3,4-dihydro-isoquinoline and 2-bromo-methyl-1-fluoro-3-trifluoromethyl-benzene.

LC-MS: rt=0.79 min, 384 (M+1, ES+).

### (S)-1-[2-(4-Chloro-3-trifluoromethyl-phenyl)-ethyl]-6,7-dimethoxy-1,2,3,4-tetrahydro-isoquinoline

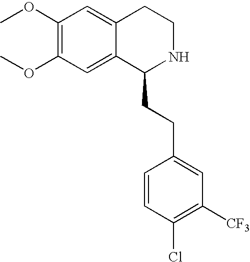

prepared from 6,7-dimethoxy-1-methyl-3,4-dihydro-isoquinoline and 4-bromo-methyl-1-chloro-2-trifluoromethyl-benzene.

LC-MS: rt=0.85 min, 400 (M+1, ES+).

### (S)-1-[2-(2,3-Difluoro-4-methyl-phenyl)-ethyl]-6,7-dimethoxy-1,2,3,4-tetrahydro-isoquinoline

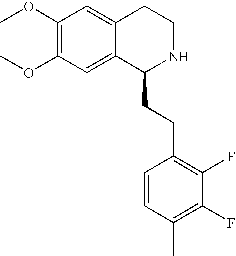

prepared from 6,7-dimethoxy-1-methyl-3,4-dihydro-isoquinoline and 1-bromo-methyl-2,3-difluoro-4-methyl-benzene.

LC-MS: rt=0.80 min, 348 (M+1, ES+).

### (S)-1-[2-(4-Difluoromethoxy-phenyl)-ethyl]-6,7-dimethoxy-1,2,3,4-tetrahydro-isoquinoline

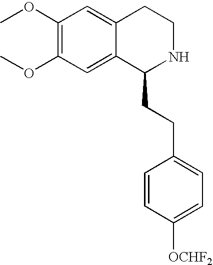

prepared from 6,7-dimethoxy-1-methyl-3,4-dihydro-isoquinoline and 1-bromo-methyl-4-difluoromethoxy-benzene.

LC-MS: rt=0.79 min, 364 (M+1, ES+).

### (S)-1-[2-(3,4-Dimethyl-phenyl)-ethyl]-6,7-dimethoxy-1,2,3,4-tetrahydro-isoquinoline

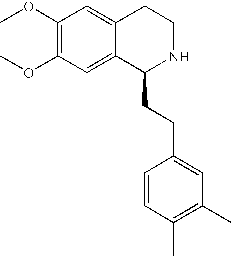

prepared from 6,7-dimethoxy-1-methyl-3,4-dihydro-isoquinoline and 4-chloro-methyl-1,2-dimethyl-benzene.

LC-MS: rt=0.79 min, 326 (M+1, ES+).

### (S)-1-[2-(3-Chloro-4-methyl-phenyl)-ethyl]-6,7-dimethoxy-1,2,3,4-tetrahydro-isoquinoline

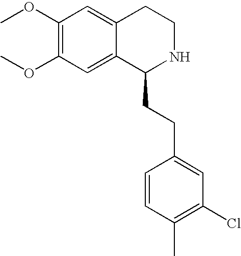

prepared from 6,7-dimethoxy-1-methyl-3,4-dihydro-isoquinoline and 2-chloro-4-chloromethyl-1-methyl-benzene.

LC-MS: rt=0.79 min, 346 (M+1, ES+).

### (S)-1-[2-(2-Chloro-6-fluoro-phenyl)-ethyl]-6,7-dimethoxy-1,2,3,4-tetrahydro-isoquinoline

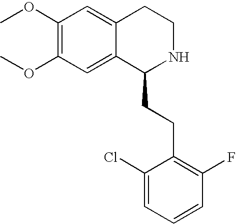

prepared from 6,7-dimethoxy-1-methyl-3,4-dihydro-isoquinoline and 1-chloro-2-chloromethyl-3-fluoro-benzene.

LC-MS: rt=0.76 min, 350 (M+1, ES+).

### (S)-6,7-Dimethoxy-1-(2-o-tolyl-ethyl)-1,2,3,4-tetrahydro-isoquinoline

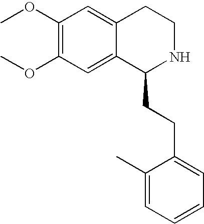

prepared from 6,7-dimethoxy-1-methyl-3,4-dihydro-isoquinoline and 1-bromo-methyl-2-methyl-benzene.

LC-MS: rt=0.77 min, 312 (M+1, ES+).

### (S)-6,7-Dimethoxy-1-(2-m-tolyl-ethyl)-1,2,3,4-tetrahydro-isoquinoline

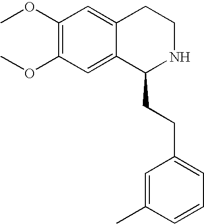

prepared from 6,7-dimethoxy-1-methyl-3,4-dihydro-isoquinoline and 1-bromo-methyl-3-methyl-benzene.

LC-MS: rt=0.78 min, 312 (M+1, ES+).

### (S)-6,7-Dimethoxy-1-(2-p-tolyl-ethyl)-1,2,3,4-tetrahydro-isoquinoline

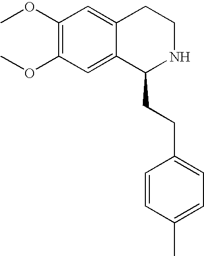

prepared from 6,7-dimethoxy-1-methyl-3,4-dihydro-isoquinoline and 1-bromo-methyl-4-methyl-benzene.

LC-MS: rt=0.78 min, 312 (M+1, ES+).

### (S)-1-[2-(2-Fluoro-phenyl)-ethyl]-6,7-dimethoxy-1,2,3,4-tetrahydro-isoquinoline

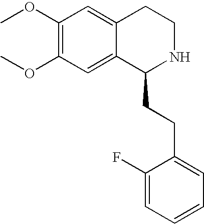

prepared from 6,7-dimethoxy-1-methyl-3,4-dihydro-isoquinoline and 1-bromo-methyl-2-fluoro-benzene.

LC-MS: rt=0.74 min, 316 (M+1, ES+).

### (S)-1-[2-(3-Fluoro-phenyl)-ethyl]-6,7-dimethoxy-1,2,3,4-tetrahydro-isoquinoline

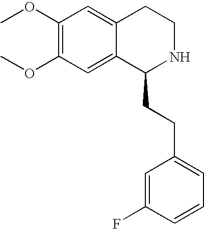

prepared from 6,7-dimethoxy-1-methyl-3,4-dihydro-isoquinoline and 1-bromo-methyl-3-fluoro-benzene.

LC-MS: rt=0.75 min, 316 (M+1, ES+).

### (S)-1-[2-(2,6-Dichloro-phenyl)-ethyl]-6,7-dimethoxy-1,2,3,4-tetrahydro-isoquinoline

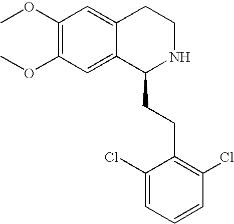

prepared from 6,7-dimethoxy-1-methyl-3,4-dihydro-isoquinoline and 1,3-dichloro-2-chloromethyl-benzene.

LC-MS: rt=0.79 min, 366 (M+1, ES+).

### (S)-1-[2-(3,4-Dichloro-phenyl)-ethyl]-6,7-dimethoxy-1,2,3,4-tetrahydro-isoquinoline

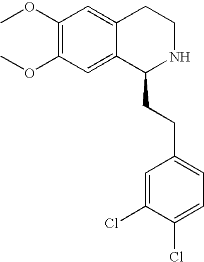

prepared from 6,7-dimethoxy-1-methyl-3,4-dihydro-isoquinoline and 1,2-dichloro-4-chloromethyl-benzene.

LC-MS: rt=1.19 min, 366 (M+1, ES+).

### (S)-1-[2-(3,5-Dimethyl-phenyl)-ethyl]-6,7-dimethoxy-1,2,3,4-tetrahydro-isoquinoline

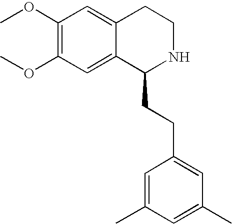

prepared from 6,7-dimethoxy-1-methyl-3,4-dihydro-isoquinoline and 1-bromo-methyl-3,5-dimethyl-benzene.

LC-MS: rt=1.15 min, 326 (M+1, ES+).

### (S)-6,7-Dimethoxy-1-[2-(2-trifluoromethyl-phenyl)-ethyl]-1,2,3,4-tetrahydro-isoquinoline

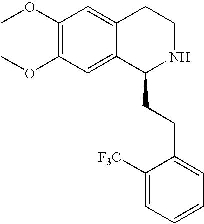

prepared from 6,7-dimethoxy-1-methyl-3,4-dihydro-isoquinoline and 1-bromo-methyl-2-trifluoromethyl-benzene.

LC-MS: rt=1.11 min, 366 (M+1, ES+).

### (S)-1-[2-(2,4-Difluoro-phenyl)-ethyl]-6,7-dimethoxy-1,2,3,4-tetrahydro-isoquinoline

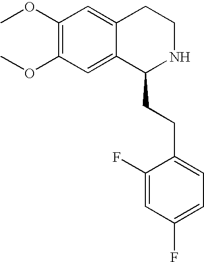

prepared from 6,7-dimethoxy-1-methyl-3,4-dihydro-isoquinoline and 1-bromo-methyl-2,4-difluoro-benzene.

LC-MS: rt=0.85 min, 334 (M+1, ES+).

### (S)-6,7-Dimethoxy-1-[2-(2,3,6-trifluoro-phenyl)-ethyl]-1,2,3,4-tetrahydro-isoquinoline

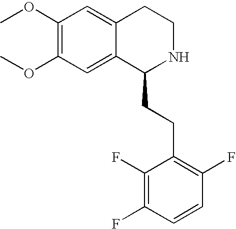

prepared from 6,7-dimethoxy-1-methyl-3,4-dihydro-isoquinoline and 2-bromo-methyl-1,3,4-trifluoro-benzene.

LC-MS: rt=0.84 min, 352 (M+1, ES+).

### (S)-6,7-Dimethoxy-1-[2-(4-trifluoromethoxy-phenyl)-ethyl]-1,2,3,4-tetrahydro-isoquinoline

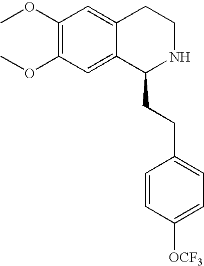

prepared from 6,7-dimethoxy-1-methyl-3,4-dihydro-isoquinoline and 1-bromo-methyl-4-trifluoromethoxy-benzene.

LC-MS: rt=0.93 min, 382 (M+1, ES+).

### (S)-1-[2-(2-Fluoro-3-methyl-phenyl)-ethyl]-6,7-dimethoxy-1,2,3,4-tetrahydro-isoquinoline

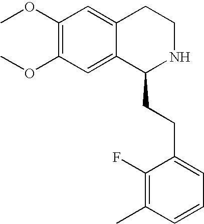

prepared from 6,7-dimethoxy-1-methyl-3,4-dihydro-isoquinoline and 1-bromo-methyl-2-fluoro-3-methyl-benzene.

LC-MS: rt=0.86 min, 330 (M+1, ES+).

### (S)-1-[2-(4-Chloro-phenyl)-ethyl]6,7-dimethoxy-1,2,3,4-tetrahydro-isoquinoline

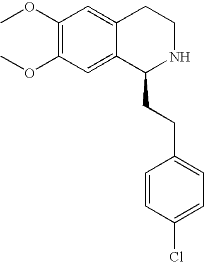

prepared from 6,7-dimethoxy-1-methyl-3,4-dihydro-isoquinoline and 1-bromo-methyl-4-chloro-benzene.

LC-MS: rt=0.88 min, 332 (M+1, ES+).

### (S)-1-[2-(3-Chloro-phenyl)-ethyl]-6,7-dimethoxy-1,2,3,4-tetrahydro-isoquinoline

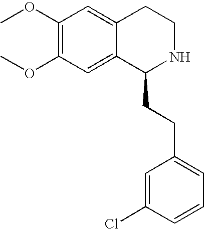

prepared from 6,7-dimethoxy-1-methyl-3,4-dihydro-isoquinoline and 1-bromo-methyl-3-chloro-benzene.

LC-MS: rt=0.88 min, 332 (M+1, ES+).

### (S)-6,7-Dimethoxy-1-[2-(3-trifluoromethoxy-phenyl)-ethyl]-1,2,3,4-tetrahydro-isoquinoline

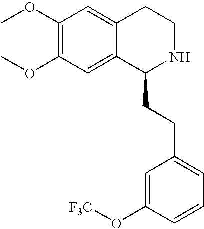

prepared from 6,7-dimethoxy-1-methyl-3,4-dihydro-isoquinoline and 1-bromo-methyl-3-trifluoromethoxy-benzene.

LC-MS: rt=0.92 min, 382 (M+1, ES+).

### (S)-6,7-Dimethoxy-1-[2-(3,4,5-trifluoro-phenyl)-ethyl]-1,2,3,4-tetrahydro-isoquinoline

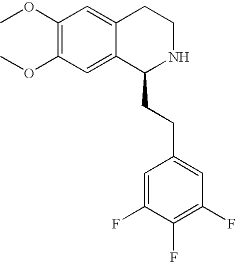

prepared from 6,7-dimethoxy-1-methyl-3,4-dihydro-isoquinoline and 5-bromo-methyl-1,2,3-trifluoro-benzene.

LC-MS: rt=0.88 min, 352 (M+1, ES+).

### (S)-6,7-Dimethoxy-1-[2-(3-trifluoromethyl-phenyl)-ethyl]-1,2,3,4-tetrahydro-isoquinoline

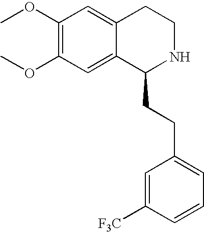

prepared from 6,7-dimethoxy-1-methyl-3,4-dihydro-isoquinoline and 1-bromo-methyl-3-trifluoromethyl-benzene.

LC-MS: rt=0.88 min, 366 (M+1, ES+).

### (S)-6,7-Dimethoxy-1-[2-(2,3,4-trifluoro-phenyl)-ethyl]-1,2,3,4-tetrahydro-isoquinoline

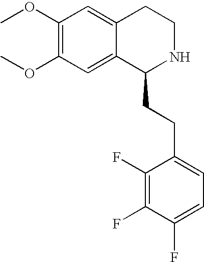

prepared from 6,7-dimethoxy-1-methyl-3,4-dihydro-isoquinoline and 1-bromo-methyl-2,3,4-trifluoro-benzene.

LC-MS: rt=0.86 min, 352 (M+1, ES+).

### (S)-1-[2-(4-Bromo-2-fluoro-phenyl)-ethyl]-6,7-dimethoxy-1,2,3,4-tetrahydro-isoquinoline

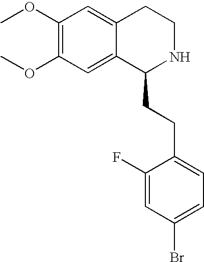

prepared from 6,7-dimethoxy-1-methyl-3,4-dihydro-isoquinoline and 4-bromo-1-bromomethyl-2-fluoro-benzene.

LC-MS: rt=0.90 min, 394 (M+1, ES+).

### (S)-1-[2-(2,6-Difluoro-phenyl)-ethyl]-6,7-dimethoxy-1,2,3,4-tetrahydro-isoquinoline

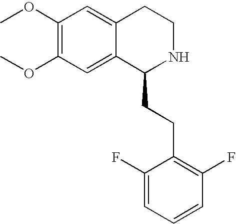

prepared from 6,7-dimethoxy-1-methyl-3,4-dihydro-isoquinoline and 2-bromo-methyl-1,3-difluoro-benzene.

LC-MS: rt=0.82 min, 334 (M+1, ES+).

### (S)-6,7-Dimethoxy-1-[2-(2-trifluoromethoxy-phenyl)-ethyl]-1,2,3,4-tetrahydro-isoquinoline

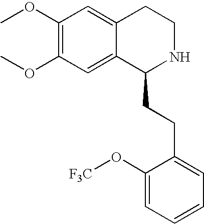

prepared from 6,7-dimethoxy-1-methyl-3,4-dihydro-isoquinoline and 1-bromo-methyl-2-trifluoromethoxy-benzene.

LC-MS: rt=0.90 min, 382 (M+1, ES+).

### (S)-1-[2-(2,4-Dichloro-phenyl)-ethyl]-6,7-dimethoxy-1,2,3,4-tetrahydro-isoquinoline

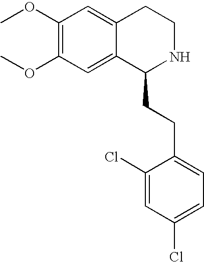

prepared from 6,7-dimethoxy-1-methyl-3,4-dihydro-isoquinoline and 2,4-dichloro-1-chloromethyl-benzene.

LC-MS: rt=0.92 min, 366 (M+1, ES+).

### (S)-6,7-Dimethoxy-1-[2-(2,4,5-trifluoro-phenyl)-ethyl]-1,2,3,4-tetrahydro-isoquinoline

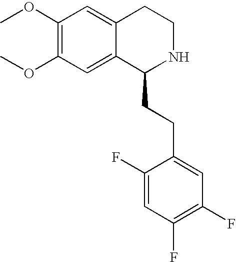

prepared from 6,7-dimethoxy-1-methyl-3,4-dihydro-isoquinoline and 1-bromo-methyl-2,4,5-trifluoro-benzene.

LC-MS: rt=0.86 min, 352 (M+1, ES+).

### (S)-1-[2-(3-Bromo-phenyl)-ethyl]-6,7-dimethoxy-1,2,3,4-tetrahydro-isoquinoline

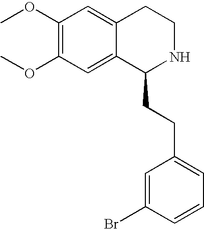

prepared from 6,7-dimethoxy-1-methyl-3,4-dihydro-isoquinoline and 1-bromo-3-bromomethyl-benzene.

LC-MS: rt=0.89 min, 376 (M+1, ES+).

### (S)-1-[2-(4-tert-Butyl-phenyl)-ethyl]-6,7-dimethoxy-1,2,3,4-tetrahydro-isoquinoline

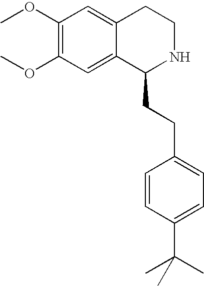

prepared from 6,7-dimethoxy-1-methyl-3,4-dihydro-isoquinoline and 1-bromo-methyl-4-tert-butyl-benzene.

LC-MS: rt=0.98 min, 354 (M+1, ES+).

### (S)-1-[2-(2-Bromo-phenyl)-ethyl]-6,7-dimethoxy-1,2,3,4-tetrahydro-isoquinoline

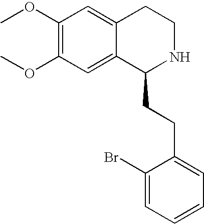

prepared from 6,7-dimethoxy-1-methyl-3,4-dihydro-isoquinoline and 1-bromo-2-bromomethyl-benzene.

LC-MS: rt=0.87 min, 376 (M+1, ES+).

### (S)-1-[2-(4-Bromo-phenyl)-ethyl]-6,7-dimethoxy-1,2,3,4-tetrahydro-isoquinoline

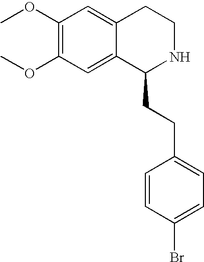

prepared from 6,7-dimethoxy-1-methyl-3,4-dihydro-isoquinoline and 1-bromo-4-bromomethyl-benzene.

LC-MS: rt=0.89 min, 376 (M+1, ES+).

D Synthesis of (3,4-Dihydro-1H-isoquinolin-2-yl)-acetic Acid Derivatives:

D.1 Synthesis of Acetic Acid Methyl Ester Derivatives (General Procedure):

Potassium tert-butoxide (5.0 mmol) is added to a suspension of the respective tetrahydroisoquinoline hydrochloride (5.0 mmol) in THF (50 mL). After 5 min DIPEA (10.0 mmol) and the respective methyl bromoacetate (5.0 mmol) are added. The reaction mixture is stirred at 60° C. for 14 h. Water (80 mL) and ethyl acetate (150 mL) are added, the layers are separated and the aqueous layer is extracted three times with ethyl acetate (50 mL each). The combined organic extracts are dried with Na₂SO₄ and the solvent is removed in vacuo. The following esters that are used in the next step without further purification are obtained as mixtures of racemic diastereoisomers:

### (6,7-Dimethoxy-1-naphthalen-2-ylmethyl-3,4-dihydro-1H-isoquinolin-2-yl)-phenyl-acetic acid methyl ester

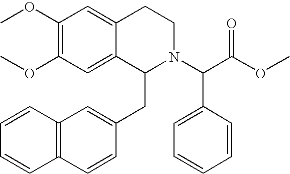

prepared by reaction of 6,7-dimethoxy-1-naphthalen-2-ylmethyl-1,2,3,4-tetrahydroisoquinoline with methyl α-bromophenylacetate.

LC-MS: rt=0.89 min (dia 1), 0.92 min (dia 2), 482 (M+1, ES+).

### {1-[2-(3,4-Difluoro-phenyl)-ethyl]-6,7-dimethoxy-3,4-dihydro-1H-isoquinolin-2-yl}-phenyl-acetic acid methyl ester

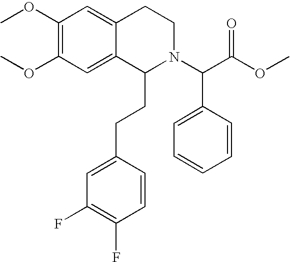

prepared by reaction of 1-[2-(3,4-difluoro-phenyl)-ethyl]-6,7-dimethoxy-1,2,3,4-tetrahydroisoquinoline with methyl α-bromophenylacetate.

LC-MS: rt=0.89 min, 482 (M+1, ES+).

### [1-(2-Furan-2-yl-ethyl)-6,7-dimethoxy-3,4-dihydro-1H-isoquinolin-2-yl]-phenyl-acetic acid methyl ester

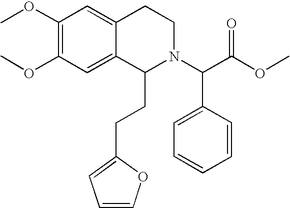

prepared by reaction of 1-(2-furan-2-yl-ethyl)-6,7-dimethoxy-1,2,3,4-tetrahydro-isoquinoline with methyl α-bromophenylacetate.

LC-MS: rt=4.53 min, 436 (M+1, ES+).

### [6,7-Dimethoxy-1-(3-phenyl-propyl)-3,4-dihydro-1H-isoquinolin-2-yl]-phenyl-acetic acid methyl ester

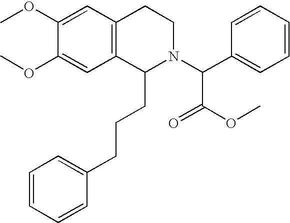

prepared by reaction of 6,7-dimethoxy-1-(3-phenyl-propyl)-1,2,3,4-tetrahydro-isoquinoline with methyl α-bromophenylacetate.

LC-MS: dia1: rt=5.04, 460 (M+1, ES+); dia2: rt=5.44, 460 (M+1, ES+).

D.2 Synthesis of Acetic Acid Derivatives (General Procedure):

A solution of the respective ester (5.0 mmol) in MeOH (200 mL) is treated with sodium hydroxide solution (1.0 M, 30 mL), stirred at 60° C. for 16 h and concentrated in vacuo to a volume of about 40 mL. Sodium hydroxide solution (1.0 M, 50 mL) and ethyl acetate (100 mL) are added, the layers are separated and the aqueous layer is extracted three times with ethyl acetate (100 mL each). The combined organic extracts are concentrated in vacuo. The residue is purified either by crystallization from ethyl acetate or by prep. HPLC to give the following acetic acid derivatives as mixtures of racemic diastereoisomers:

### (6,7-Dimethoxy-1-naphthalen-2-ylmethyl-3,4-dihydro-1H-isoquinolin-2-yl)-phenyl-acetic acid

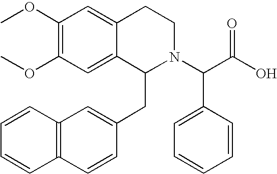

prepared by saponification of (6,7-dimethoxy-1-naphthalen-2-ylmethyl-3,4-dihydro-1H-isoquinolin-2-yl)-phenyl-acetic acid methyl ester.

LC-MS: rt=0.83 min (dia 1), 0.85 min (dia 2), 468 (M+1, ES+).

### {1-[2-(3,4-Difluoro-phenyl)-ethyl]-6,7-dimethoxy-3,4-dihydro-1H-isoquinolin-2-yl}-phenyl-acetic acid

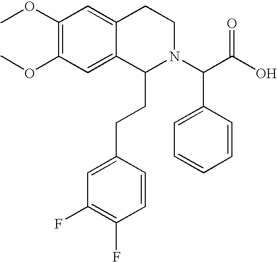

prepared by saponification of {1-[2-(3,4-difluoro-phenyl)-ethyl]-6,7-dimethoxy-3,4-dihydro-1H-isoquinolin-2-yl}-phenyl-acetic acid methyl ester.

LC-MS: rt=0.83 min, 468 (M+1, ES+).

### [1-(2-Furan-2-yl-ethyl)-6,7-dimethoxy-3,4-dihydro-1H-isoquinolin-2-yl]-phenyl-acetic acid

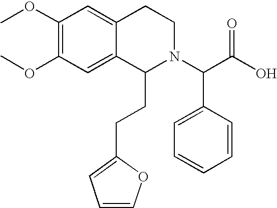

prepared by saponification of [1-(2-furan-2-yl-ethyl)-6,7-dimethoxy-3,4-dihydro-1H-isoquinolin-2-yl]-phenyl-acetic acid methyl ester.

LC-MS: rt=3.68 min, 422 (M+1, ES+).

### [6,7-Dimethoxy-1-(3-phenyl-propyl)-3,4-dihydro-1H-isoquinolin-2-yl]-phenyl-acetic acid

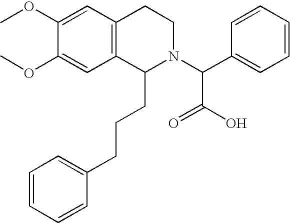

prepared by saponification of [6,7-dimethoxy-1-(3-phenyl-propyl)-3,4-dihydro-1H-isoquinolin-2-yl]-phenyl-acetic acid methyl ester.

LC-MS: rt=4.14, 446 (M+1, ES+).

D.3 One-Pot Synthesis of Acetic Acid Derivatives (General Procedure):

A solution of potassium tert-butoxide (0.30 mmol) in THF (0.50 mL) is added to the respective tetrahydroisoquinoline hydrochloride (0.30 mmol). After 5 min DIPEA (0.60 mmol) and a solution of the respective methyl bromoacetate (0.30 mmol) in THF (0.50 mL) are added. The reaction mixture is stirred at 60° C. for 14 h. An aqueous solution of sodium hydroxide (3.0 mmol, 1.5 mL) and ethanol (0.5 mL) are added and the mixture is shaken vigorously at 60° C. over night. The layers are separated, the solvent is removed in vacuo and the residue is purified by prep. HPLC to give the following acetic acid derivatives as mixtures of racemic diastereoisomers:

### (1-Benzyl-6-methoxy-3,4-dihydro-1H-isoquinolin-2-yl)-phenyl-acetic acid

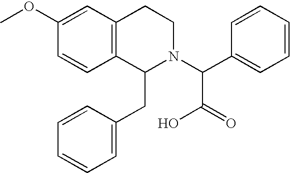

prepared by reaction of 1-benzyl-6-methoxy-1,2,3,4-tetrahydro-isoquinoline with methyl α-bromophenylacetate.

LC-MS: rt=0.81 min (dia 1), 0.82 min (dia 2), 388 (M+1, ES+).

### {1-[2-(2,3-Difluoro-phenyl)-ethyl]-6,7-dimethoxy-3,4-dihydro-1H-isoquinolin-2-yl}-phenyl-acetic acid

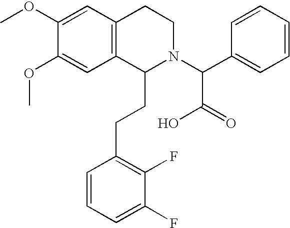

prepared by reaction of 1-[2-(2,3-difluoro-phenyl)-ethyl]-6,7-dimethoxy-1,2,3,4-tetrahydro-isoquinoline with methyl α-bromophenylacetate.

LC-MS: rt=0.83 min, 468 (M+1, ES+).

### [1-(4-Fluoro-benzyl)-6,7-dimethoxy-3,4-dihydro-1H-isoquinolin-2-yl]-phenyl-acetic acid

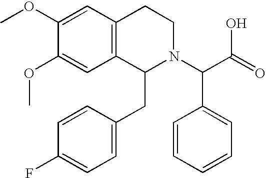

prepared by reaction of 1-(4-fluoro-benzyl)-6,7-dimethoxy-1,2,3,4-tetrahydro-isoquinoline with methyl α-bromophenylacetate.

LC-MS: rt=0.78 min (dia 1), 0.79 min (dia 2), 436 (M+1, ES+).

### [1-(2,5-Dimethoxy-benzyl)-6,7-dimethoxy-3,4-dihydro-1H-isoquinolin-2-yl]-phenyl-acetic acid

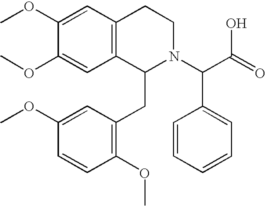

prepared by reaction of 1-(2,5-dimethoxy-benzyl)-6,7-dimethoxy-1,2,3,4-tetrahydro-isoquinoline with methyl α-bromophenylacetate.

LC-MS: rt=0.78 min (dia 1), 0.82 min (dia 2), 478 (M+1, ES+).

### {6,7-Dimethoxy-1-[2-(4-trifluoromethyl-phenyl)-ethyl]-3,4-dihydro-1H-isoquinolin-2-yl}-phenyl-acetic acid

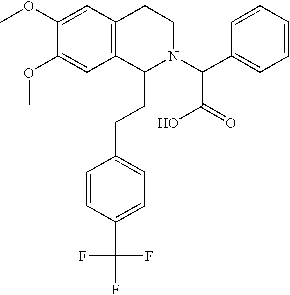

prepared by reaction of 6,7-dimethoxy-1-[2-(4-trifluoromethyl-phenyl)-ethyl]-1,2,3,4-tetrahydro-isoquinoline with methyl α-bromophenylacetate.

LC-MS: rt=0.88 min, 500 (M+1, ES+).

### {6,7-Dimethoxy-1-[2-(3-methoxy-phenyl)-ethyl]-3,4-dihydro-1H-isoquinolin-2-yl}-phenyl-acetic acid

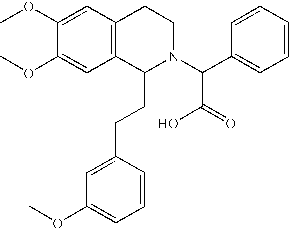

prepared by reaction of 6,7-dimethoxy-1-[2-(3-methoxy-phenyl)-ethyl]-1,2,3,4-tetrahydro-isoquinoline with methyl α-bromophenylacetate.

LC-MS: rt=0.81 min, 462 (M+1, ES+).

### [6,7-Dimethoxy-1-(4-methoxy-benzyl)-3,4-dihydro-1H-isoquinolin-2-yl]-phenyl-acetic acid

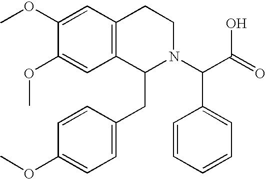

prepared by reaction of 6,7-dimethoxy-1-(4-methoxy-benzyl)-1,2,3,4-tetrahydro-isoquinoline with methyl α-bromophenylacetate.

LC-MS: rt=0.77 min (dia 1), 0.79 min (dia 2), 448 (M+1, ES+).

### {1-[2-(2,5-Difluoro-phenyl)-ethyl]-6,7-dimethoxy-3,4-dihydro-1H-isoquinolin-2-yl}-phenyl-acetic acid

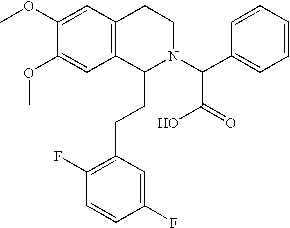

prepared by reaction of 1-[2-(2,5-difluoro-phenyl)-ethyl]-6,7-dimethoxy-1,2,3,4-tetrahydro-isoquinoline with methyl α-bromophenylacetate.

LC-MS: rt=0.83 min, 468 (M+1, ES+).

### {6,7-Dimethoxy-1-[2-(2-methoxy-phenyl)-ethyl]-3,4-dihydro-1H-isoquinolin-2-yl}-phenyl-acetic acid

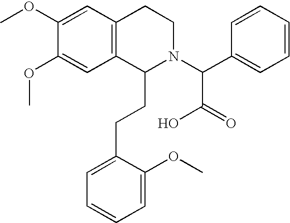

prepared by reaction of 6,7-dimethoxy-1-[2-(2-methoxy-phenyl)-ethyl]-1,2,3,4-tetrahydro-isoquinoline with methyl α-bromophenylacetate.

LC-MS: rt=0.83 min, 462 (M+1, ES+).

### {1-[2-(4-Fluoro-phenyl)-ethyl]-6,7-dimethoxy-3,4-dihydro-1H-isoquinolin-2-yl}-phenyl-acetic acid

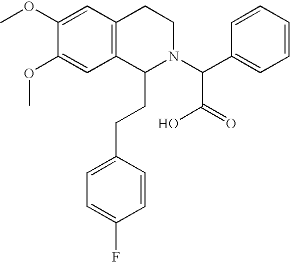

prepared by reaction of 1-[2-(4-fluoro-phenyl)-ethyl]-6,7-dimethoxy-1,2,3,4-tetrahydro-isoquinoline with methyl α-bromophenylacetate.

LC-MS: rt=0.82 min, 450 (M+1, ES+).

### {1-[2-(2,3-Difluoro-phenyl)-vinyl]-6,7-dimethoxy-3,4-dihydro-1H-isoquinolin-2-yl}-phenyl-acetic acid

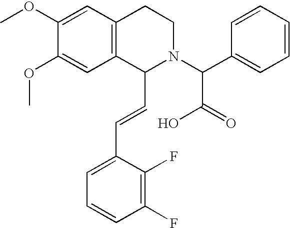

prepared by reaction of 1-[2-(2,3-difluoro-phenyl)-vinyl]-6,7-dimethoxy-1,2,3,4-tetrahydro-isoquinoline with methyl α-bromophenylacetate.

LC-MS: rt=0.83 min, 466 (M+1, ES+).

### [1-(3-Fluoro-4-methoxy-benzyl)-6,7-dimethoxy-3,4-dihydro-1H-isoquinolin-2-yl]-phenyl-acetic acid

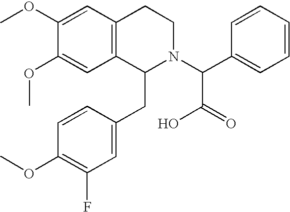

prepared by reaction of 1-(3-fluoro-4-methoxy-benzyl)-6,7-dimethoxy-1,2,3,4-tetrahydro-isoquinoline with methyl α-bromophenylacetate.

LC-MS: rt=0.77 min (dia 1), 0.79 min (dia 2), 466 (M+1, ES+).

### {1-[2-(3,4-Dimethoxy-phenyl)-ethyl]-6,7-dimethoxy-3,4-dihydro-1H-isoquinolin-2-yl}-phenyl-acetic acid

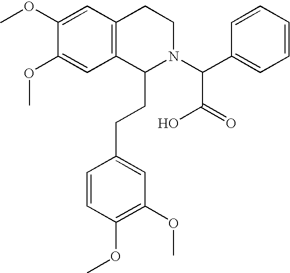

prepared by reaction of 1-[2-(3,4-dimethoxy-phenyl)-ethyl]-6,7-dimethoxy-1,2,3,4-tetrahydro-isoquinoline with methyl α-bromophenylacetate.

LC-MS: rt=0.77 min, 492 (M+1, ES+).

### [1-(2,5-Dimethoxy-benzyl)-5,8-dimethoxy-3,4-dihydro-1-isoquinolin-2-yl]-phenyl-acetic acid

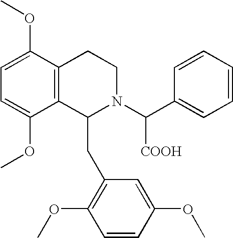

prepared by reaction of 1-(2,5-dimethoxy-benzyl)-5,8-dimethoxy-1,2,3,4-tetrahydro-isoquinoline with methyl α-bromophenylacetate.

LC-MS: rt=0.86 min (dia 1), 0.89 min (dia 2), 478 (M+1, ES+).

### {1-[2-(2,5-Difluoro-phenyl)-vinyl]-6,7-dimethoxy-3,4-dihydro-1H-isoquinolin-2-yl}-phenyl-acetic acid

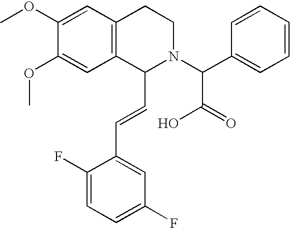

prepared by reaction of 1-[2-(2,5-difluoro-phenyl)-vinyl]-6,7-dimethoxy-1,2,3,4-tetrahydro-isoquinoline with methyl α-bromophenylacetate.

LC-MS: rt=0.83 min, 466 (M+1, ES+).

### (6,7-Dimethoxy-1-phenoxymethyl-3,4-dihydro-1H-isoquinolin-2-yl)-phenyl-acetic acid

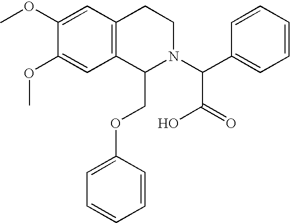

prepared by reaction of 6,7-dimethoxy-1-phenoxymethyl-1,2,3,4-tetrahydro-isoquinoline with methyl α-bromophenylacetate.

LC-MS: rt=0.80 min (dia 1), 0.81 min (dia 2), 434 (M+1, ES+).

### [6,7-Dimethoxy-1-(3-methoxy-benzyl)-3,4-dihydro-1H-isoquinolin-2-yl]-phenyl-acetic acid

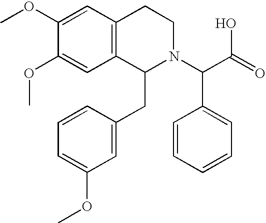

prepared by reaction of 6,7-dimethoxy-1-(3-methoxy-benzyl)-1,2,3,4-tetrahydro-isoquinoline with methyl α-bromophenylacetate.

LC-MS: rt=0.77 min (dia 1), 0.79 min (dia 2), 448 (M+1, ES+).

### [6-(3,4-Dimethoxy-benzyl)-2,3,8,9-tetrahydro-6H-[1,4]dioxino[2,3-g]isoquinolin-7-yl]-phenyl-acetic acid

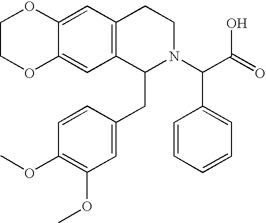

prepared by reaction of 6-(3,4-dimethoxy-benzyl)-2,3,6,7,8,9-hexahydro-[1,4]dioxino[2,3-g]isoquinoline with methyl α-bromophenylacetate.

LC-MS: rt=0.77 min (dia 1), 0.79 min (dia 2), 476 (M+1, ES+).

E Synthesis of (3,4-Dihydro-1H-isoquinolin-2-yl)-acetonitrile Derivatives (General Procedure):

To a solution of sodium metabisulfite (5.7 mmol) in water (20 mL) is added the respective aldehyde (10.7 mmol), methanol (1.5 mL) and sodium cyanide (11.0 mmol). After 15 min a solution of the respective tetrahydroisoquinoline (10.7 mmol) in methanol (15 mL) is added and the mixture is stirred for 3 h. Water (50 mL) and ethyl acetate (50 mL) are added, the layers are separated and the aqueous layer is extracted twice with ethyl acetate (50 mL each). The combined organic extracts are concentrated in vacuo to give the following acetonitrile derivatives which are used in the next step without further purification as mixtures of racemic diastereoisomers:

### (2-Chloro-phenyl)-[1-(3,4-dimethoxy-benzyl)-6,7-dimethoxy-3,4-dihydro-1H-isoquinolin-2-yl]-acetonitrile

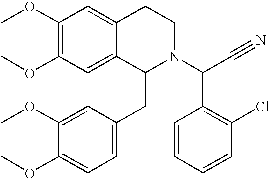

prepared by reaction of tetrahydropapaverine with 2-chlorobenzaldehyde.

LC-MS: rt=1.23 min, 493 (M+1, ES+).

F Synthesis of Tosylated Mandelamide Derivatives:

F.1 Synthesis of Toluene-4-sulfonic Acid carbamoyl-phenyl-methyl Ester:

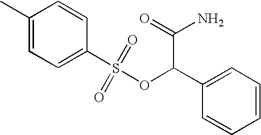

To a suspension of mandelamide (25 mmol) and DMAP (2.0 mmol) in DCM (200 mL) DIPEA (27.5 mmol) is added. The mixture is treated portion wise with p-toluenesulfonyl chloride (27.5 mmol) and stirred for 20 h. The solvents are removed in vacuo. The residue is diluted with ethyl acetate (350 mL) and washed three times with sat. NaHCO₃ solution (3×150 mL) and once with brine (100 mL). The organic extracts are dried with Na₂SO₄ and concentrated in vacuo. The residue is recrystallized from ethyl acetate/heptane 4/1 to give the desired tosylate as white crystals.

LC-MS: rt=0.87 min, 306 (M+1, ES+).
F.2 Synthesis of (S)-2-Hydroxy-2-phenyl-acetamide:

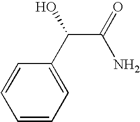

Methyl(S)-(+)-mandelate (120 mmol) is dissolved in a solution of ammonia in methanol (7.0 M, 1.2 mol) at RT. After 1 d nitrogen is bubbled through the solution for 30 min and the solvents are removed in vacuo to give the desired amide as a white solid.

LC-MS: rt=0.33 min, 152 (M+1, ES+).
F.3 Synthesis of (S)-Toluene-4-sulfonic Acid carbamoyl-phenyl-methyl Ester:

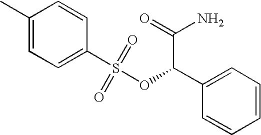

To a solution of (S)-2-Hydroxy-2-phenyl-acetamide (120 mmol) in dioxane (200 mL) are added DIPEA (133 mmol) and DMAP (10 mmol). A solution of p-toluenesulfonyl chloride (121 mmol) in dioxane (50 mL) is added at RT. After 1 d ethyl acetate (400 mL) is added and the organic layer is washed several times with sat. NaHCO₃ solution. Silica gel (50 g) is added, the solvents are removed in vacuo and the residue is purified by flash chromatography (ethyl acetate/heptane 1:2) to give the desired tosylate.

LC-MS: rt=0.87 min, 306 (M+1, ES+).

G Synthesis of Substituted and Unsubstituted Glycinamide Derivatives:

G.1 Synthesis of Unsubstituted Glycinamides (General Procedure):

To a suspension of the respective tetrahydroisoquinoline hydrochloride (0.1 mmol) in THF (0.5 mL) is added a solution of DIPEA (0.3 mmol) in THF (0.5 mL) and a solution of 2-bromoacetamide (0.1 mmol) in THF (0.5 mL). The suspension is stirred at 65° C. for 14 h, the solvent is removed in vacuo and the residue is purified by prep. HPLC to give the following amides as racemic mixtures:

### EXAMPLE 1

### 2-{1-[2-2,5-Bis-trifluoromethyl-phenyl)-ethyl]-6,7-dimethoxy-3,4-dihydro-1H-isoquinolin-2-yl}-acetamide

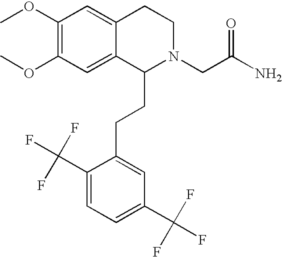

prepared by reaction of 1-[2-(2,5-bis-trifluoromethyl-phenyl)-ethyl]-6,7-dimethoxy-1,2,3,4-tetrahydroisoquinoline with 2-bromoacetamide.

LC-MS: rt=0.83 min, 491 (M+1, ES+).

G.2 Synthesis of Racemic α-Substituted Glycinamides via Acetic Acid Derivatives (General Procedure):

To a solution of the respective carboxylic acid (3.8 mmol) in DCM (40 mL) is added DMAP (15 mmol) and EDC×HCl (5.7 mmol). After 5 min ammonium bromide (5.0 mmol) is added and the reaction mixture is stirred for 14 h. Water (100 mL) is added, the layers are separated and the aqueous layer is extracted three times with ethyl acetate (100 mL each). The combined organic extracts are concentrated in vacuo and the residue is purified by prep. HPLC to give the following amide derivatives as mixtures of racemic diastereoisomers:

### EXAMPLE 2

### 2-(6,7-Dimethoxy-1-naphthalen-2-ylmethyl-3,4-dihydro-1H-isoquinolin-2-yl)-2-phenyl-acetamide

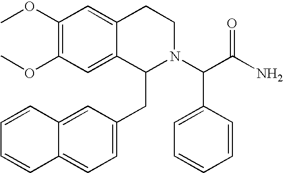

prepared by reaction of (6,7-dimethoxy-1-naphthalen-2-ylmethyl-3,4-dihydro-1H-isoquinolin-2-yl)-phenyl-acetic acid with ammonium bromide.

LC-MS: rt=0.83 min, 467 (M+1, ES+).

### EXAMPLE 3

### 2-{1-[2-(3,4-Difluoro-phenyl)-ethyl]-6,7-dimethoxy-3,4-dihydro-1H-isoquinolin-2-yl}-2-phenyl-acetamide

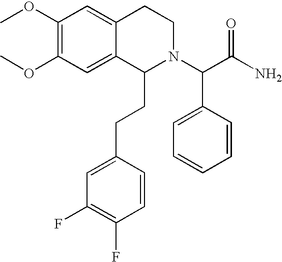

prepared by reaction of {1-[2-(3,4-difluoro-phenyl)-ethyl]-6,7-dimethoxy-3,4-dihydro-1H-isoquinolin-2-yl}-phenyl-acetic acid with ammonium bromide.

LC-MS: rt=0.81 min, 467 (M+1, ES+).

### EXAMPLE 4

### 2-[1-(2-Furan-2-yl-ethyl)-6,7-dimethoxy-3,4-dihydro-1H-isoquinolin-2-yl]-2-phenyl-acetamide

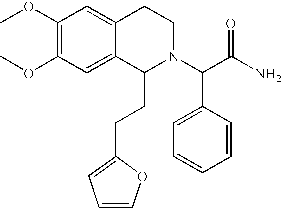

prepared by reaction of [1-(2-furan-2-yl-ethyl)-6,7-dimethoxy-3,4-dihydro-1H-isoquinolin-2-yl]-phenyl-acetic acid with ammonium bromide.

LC-MS: rt=3.54 min, 421 (M+1, ES+).

### EXAMPLE 5

### 2-(1-Benzyl-6-methoxy-3,4-dihydro-1H-isoquinolin-2-yl)-2-phenyl-acetamide

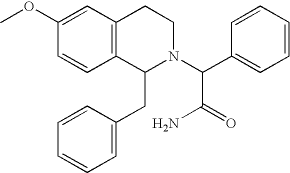

prepared by reaction of (1-benzyl-6-methoxy-3,4-dihydro-1H-isoquinolin-2-yl)-phenyl-acetic acid with ammonium bromide.

LC-MS: rt=0.82 min, 387 (M+1, ES+).

### EXAMPLE 6

### 2-{1-[2-(2,3-Difluoro-phenyl)-ethyl]-6,7-dimethoxy-3,4-dihydro-1H-isoquinolin-2-yl}-2-phenyl-acetamide

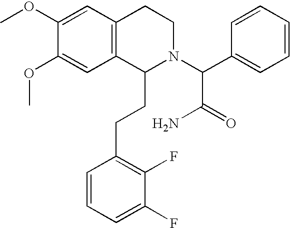

prepared by reaction of {1-[2-(2,3-difluoro-phenyl)-ethyl]-6,7-dimethoxy-3,4-dihydro-1H-isoquinolin-2-yl}-phenyl-acetic acid with ammonium bromide.

LC-MS: rt=0.83 min, 467 (M+1, ES+).

### EXAMPLE 7

### 2-[1-(4-Fluoro-benzyl)-6,7-dimethoxy-3,4-dihydro-1H-isoquinolin-2-yl]-2-phenyl-acetamide

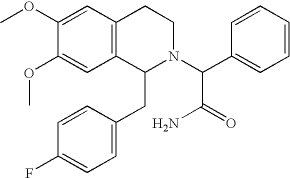

prepared by reaction of [1-(4-fluoro-benzyl)-6,7-dimethoxy-3,4-dihydro-1H-isoquinolin-2-yl]-phenyl-acetic acid with ammonium bromide.

LC-MS: rt=0.79 min, 435 (M+1, ES+).

### EXAMPLE 8

### 2-[1-(2,5-Dimethoxy-benzyl)-6,7-dimethoxy-3,4-dihydro-1H-isoquinolin-2-yl]-2-phenyl-acetamide

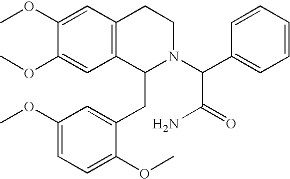

prepared by reaction of [1-(2,5-dimethoxy-benzyl)-6,7-dimethoxy-3,4-dihydro-1H-isoquinolin-2-yl]-phenyl-acetic acid with ammonium bromide.

LC-MS: rt=0.79 min (dia 1), 0.80 min (dia 2), 477 (M+1, ES+).

### EXAMPLE 9

### 2-{6,7-Dimethoxy-1-[2-(4-trifluoromethyl-phenyl)-ethyl]-3,4-dihydro-1H-isoquinolin-2-yl}-2-phenyl-acetamide

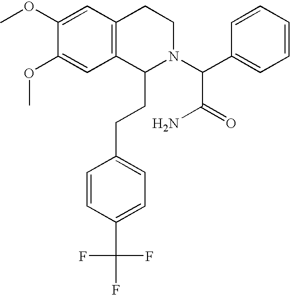

prepared by reaction of {6,7-dimethoxy-1-[2-(4-trifluoromethyl-phenyl)-ethyl]-3,4-dihydro-1H-isoquinolin-2-yl}-phenyl-acetic acid with ammonium bromide.

LC-MS: rt=0.87 min, 499 (M+1, ES+).

### EXAMPLE 10

### 2-{6,7-Dimethoxy-1-[2-(3-methoxy-phenyl)-ethyl]-3,4-dihydro-1H-isoquinolin-2-yl}-2-phenyl-acetamide

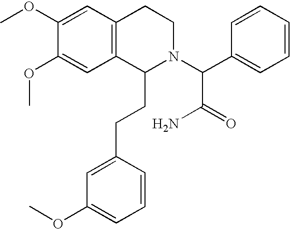

prepared by reaction of {6,7-dimethoxy-1-[2-(3-methoxy-phenyl)-ethyl]-3,4-dihydro-1H-isoquinolin-2-yl}-phenyl-acetic acid with ammonium bromide.

LC-MS: rt=0.81 min, 461 (M+1, ES+).

### EXAMPLE 11

### 2-[6,7-Dimethoxy-1-(4-methoxy-benzyl)-3,4-dihydro-1H-isoquinolin-2-yl]-2-phenyl-acetamide

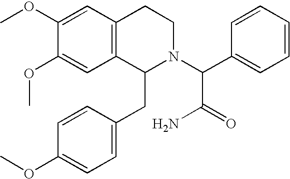

prepared by reaction of [6,7-dimethoxy-1-(4-methoxy-benzyl)-3,4-dihydro-1H-isoquinolin-2-yl]-phenyl-acetic acid with ammonium bromide.

LC-MS: rt=0.78 min, 447 (M+1, ES+).

### EXAMPLE 12

### 2-{1-[2-(2,5-Difluoro-phenyl)-ethyl]-6,7-dimethoxy-3,4-dihydro-1H-isoquinolin-2-yl}-2-phenyl-acetamide

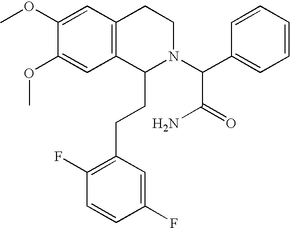

prepared by reaction of {1-[2-(2,5-difluoro-phenyl)-ethyl]-6,7-dimethoxy-3,4-dihydro-1H-isoquinolin-2-yl}-phenyl-acetic acid with ammonium bromide.

LC-MS: rt=0.83 min, 467 (M+1, ES+).

### EXAMPLE 13

### 2-{6,7-Dimethoxy-1-[2-(2-methoxy-phenyl)-ethyl]-3,4-dihydro-1H-isoquinolin-2-yl}-2-phenyl-acetamide

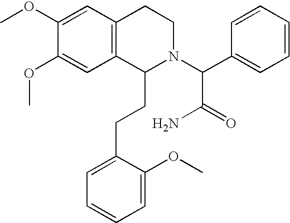

prepared by reaction of {6,7-dimethoxy-1-[2-(2-methoxy-phenyl)-ethyl]-3,4-dihydro-1H-isoquinolin-2-yl}-phenyl-acetic acid with ammonium bromide.

LC-MS: rt 0.82 min, 461 (M+1, ES+).

### EXAMPLE 14

### 2-{(1-[2-(4-Fluoro-phenyl)-ethyl]-6,7-dimethoxy-3,4-dihydro-1H-isoquinolin-2-yl}-2-phenyl-acetamide

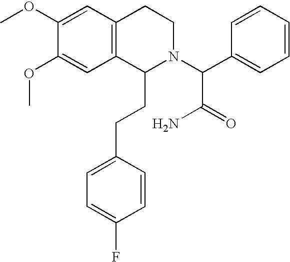

prepared by reaction of {1-[2-(4-fluoro-phenyl)-ethyl]-6,7-dimethoxy-3,4-dihydro-1H-isoquinolin-2-yl}-phenyl-acetic acid with ammonium bromide.

LC-MS: rt=0.82 min, 449 (M+1, ES+).

### EXAMPLE 15

### 2-{1-[2-2,3-Difluoro-phenyl)-vinyl]-6,7-dimethoxy-3,4-dihydro-1H-isoquinolin-2-yl}-2-phenyl-acetamide

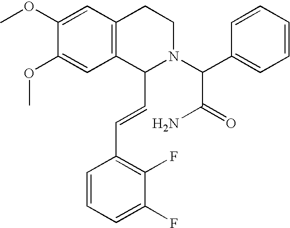

prepared by reaction of {1-[2-(2,3-difluoro-phenyl)-vinyl]-6,7-dimethoxy-3,4-dihydro-1H-isoquinolin-2-yl}-phenyl-acetic acid with ammonium bromide.

LC-MS: rt=0.82 min, 465 (M+1, ES+).

### EXAMPLE 16

### 2-[1-(3-Fluoro-4-methoxy-benzyl)-6,7-dimethoxy-3,4-dihydro-1H-isoquinolin-2-yl]-2-phenyl-acetamide

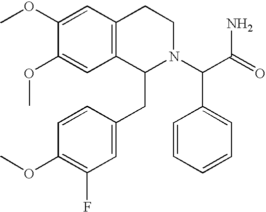

prepared by reaction of [1-(3-fluoro-4-methoxy-benzyl)-6,7-dimethoxy-3,4-dihydro-1H-isoquinolin-2-yl]-phenyl-acetic acid with ammonium bromide.

LC-MS: rt=0.79 min, 465 (M+1, ES+).

### EXAMPLE 17

### 2-{1-[2-3,4-Dimethoxy-phenyl)-ethyl]-6,7-dimethoxy-3,4-dihydro-1H-isoquinolin-2-yl}-2-phenyl-acetamide

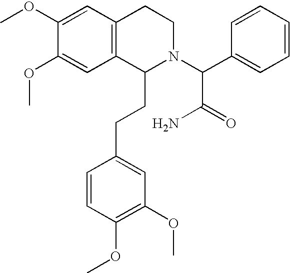

prepared by reaction of {1-[2-(3,4-dimethoxy-phenyl)-ethyl]-6,7-dimethoxy-3,4-dihydro-1H-isoquinolin-2-yl}-phenyl-acetic acid with ammonium bromide.

LC-MS: rt=0.76 min (dia 1), 0.78 min (dia 2), 491 (M+1, ES+).

### EXAMPLE 18

### 2-[1-(2,5-Dimethoxy-benzyl)-5,8-dimethoxy-3,4-dihydro-1H-isoquinolin-2-yl]-2-phenyl-acetamide

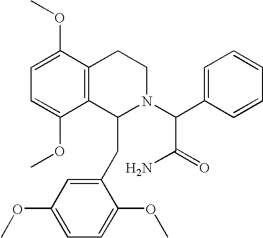

prepared by reaction of [1-(2,5-dimethoxy-benzyl)-5,8-dimethoxy-3,4-dihydro-1H-isoquinolin-2-yl]-phenyl-acetic acid with ammonium bromide.

LC-MS: rt=0.87 min, 477 (M+1, ES+).

### EXAMPLE 19

### 2-{1-[2-(2,5-Difluoro-phenyl)-vinyl]-6,7-dimethoxy-3,4-dihydro-1H-isoquinolin-2-yl}-2-phenyl-acetamide

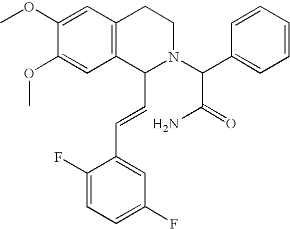

prepared by reaction of {1-[2-(2,5-difluoro-phenyl)-vinyl]-6,7-dimethoxy-3,4-dihydro-1H-isoquinolin-2-yl}1-phenyl-acetic acid with ammonium bromide.

LC-MS: rt=0.82 min, 465 (M+1, ES+).

### EXAMPLE 20

### 2-(6,7-Dimethoxy-1-phenoxymethyl-3,4-dihydro-1H-isoquinolin-2-yl)-2-phenyl-acetamide

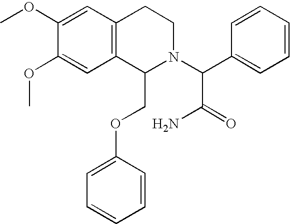

prepared by reaction of (6,7-dimethoxy-1-phenoxymethyl-3,4-dihydro-1H-isoquinolin-2-yl)-phenyl-acetic acid with ammonium bromide.

LC-MS: rt=0.81 min, 433 (M+1, ES+).

### EXAMPLE 21

### 2-[6,7-Dimethoxy-1-(3-methoxybenzyl)-3,4-dihydro-1H-isoquinolin-2-yl]-2-phenyl-acetamide

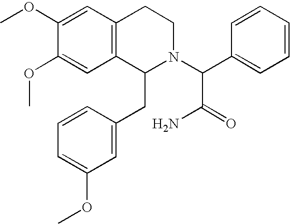

prepared by reaction of [6,7-dimethoxy-1-(3-methoxy-benzyl)-3,4-dihydro-1H-isoquinolin-2-yl]-phenyl-acetic acid with ammonium bromide.

LC-MS: rt=0.78 min (dia 1), 0.79 min (dia 2), 447 (M+1, ES+).

### EXAMPLE 22

### 2-[6-(3,4-Dimethoxy-benzyl)-2,3,8,9-tetrahydro-6H-[1,4]dioxino[2,3-g]isoquinolin-7-yl]-2-phenyl-acetamide

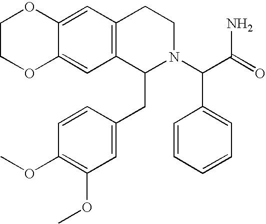

prepared by reaction of [6-(3,4-dimethoxy-benzyl)-2,3,8,9-tetrahydro-6H-[1,4]dioxino[2,3-g]isoquinolin-7-yl]-phenyl-acetic acid with ammonium bromide.

LC-MS: rt=0.77 min (dia 1), 0.78 min (dia 2), 475 (M+1, ES+).

### EXAMPLE 23

### 2-[6,7-Dimethoxy-1-(3-phenyl-propyl)-3,4-dihydro-1]-isoquinolin-2-yl}-2-phenyl-acetamide

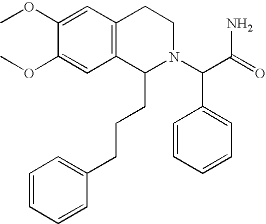

prepared by reaction of [6,7-dimethoxy-1-(3-phenyl-propyl)-3,4-dihydro-1H-isoquinolin-2-yl]-phenyl-acetic acid with ammonium bromide.

LC-MS: rt=3.83 min, 445 (M+1, ES+).

G.3 Synthesis of Racemic α-Substituted Glycinamides via Acetonitrile Derivatives (General Procedure):

The crude acetonitrile derivative (10.7 mmol) is dissolved in DMSO (25 mL). Potassium carbonate (4.6 mmol) and hydrogen peroxide solution (30%, 1.8 mL) are added and the mixture is stirred for 21 h. Water (50 mL) and ethyl acetate (100 mL) are added, the layers are separated and the aqueous layer is extracted twice with ethyl acetate (100 mL each). The combined organic extracts are concentrated in vacuo and the residue is purified by flash chromatography to give the following glycinamide derivatives as mixtures of racemic diastereoisomers:

### EXAMPLE 24

### 2-(2-Chloro-phenyl)-2-[1-(3,4-dimethoxy-benzyl)-6,7-dimethoxy-3,4-dihydro-1H-isoquinolin-2-yl]-acetamide

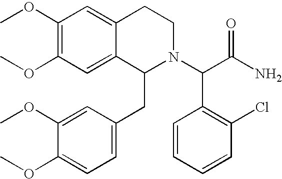

prepared by saponification of (2-chloro-phenyl)-[1-(3,4-dimethoxy-benzyl)-6,7-dimethoxy-3,4-dihydro-1H-isoquinolin-2-yl]-acetonitrile.

LC-MS: rt=0.82 min, 511 (M+1, ES+).

G.4 Synthesis of α-Substituted Glycinamides via Alkylation of Enantiomerically Enriched 1,2,3,4-tetrahydroisoquinolines with Racemic Tosylates (General Procedure):

DIPEA (0.300 mmol) and toluene-4-sulfonic acid carbamoyl-phenyl-methyl ester (0.125 mmol) are added to a solution of the respective enantiomerically enriched 1,2,3,4-tetrahydroisoquinoline derivative (0.125 mmol) in THF (2.0 mL). The mixture is refluxed for 4 d and cooled to RT. The obtained mixture of enantiomerically enriched diastereoisomers is separated by prep. HPLC to give the following amides; datas are given for the more polar (HPLC) and more active (IC₅₀, FLIPR) diastereoisomer:

### EXAMPLE 25

### (R)-2-{(S)-6,7-Dimethoxy-1-[2-(2-methyl-5-trifluoromethyl-phenyl)-ethyl]-3,4-dihydro-1H-isoquinolin-2-yl}-2-phenyl-acetamide

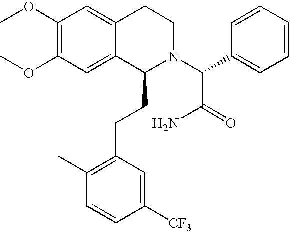

prepared by reaction of toluene-4-sulfonic acid carbamoyl-phenyl-methyl ester with (S)-6,7-dimethoxy-1-[2-(2-methyl-5-trifluoromethyl-phenyl)-ethyl]-1,2,3,4-tetrahydro-isoquinoline.

LC-MS: rt=0.90 min, 513 (M+1, ES+).

### EXAMPLE 26

### (R)-2-{(S)-1-[2-(3-Chloro-2-fluoro-phenyl)-ethyl]-6,7-dimethoxy-3,4-dihydro-1H-isoquinolin-2-yl}-2-phenyl-acetamide

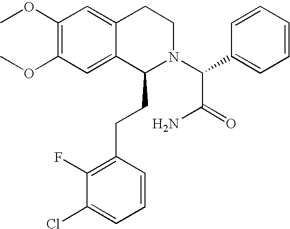

prepared by reaction of toluene-4-sulfonic acid carbamoyl-phenyl-methyl ester with (S)-1-[2-(3-chloro-2-fluoro-phenyl)-ethyl]-6,7-dimethoxy-1,2,3,4-tetrahydro-isoquinoline.

LC-MS: rt=0.86 min, 483 (M+1, ES+).

### EXAMPLE 27

### (R)-2-{(S)-1-[2-(3-Fluoro-4-methyl-phenyl)-ethyl]-6,7-dimethoxy-3,4-dihydro-1H-isoquinolin-2-yl}-2-phenyl-acetamide

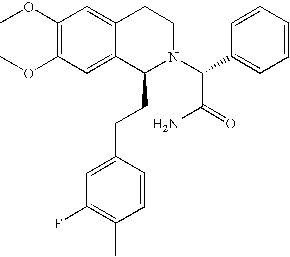

prepared by reaction of toluene-4-sulfonic acid carbamoyl-phenyl-methyl ester with (S)-1-[2-(3-fluoro-4-methyl-phenyl)-ethyl]-6,7-dimethoxy-1,2,3,4-tetrahydro-isoquinoline.

LC-MS: rt=0.85 min, 463 (M+1, ES+).

### EXAMPLE 28

### (R)-2-{(S)-1-[2-(4-Fluoro-2-trifluoromethyl-phenyl)-ethyl]-6,7-dimethoxy-3,4-dihydro-1H-isoquinolin-2-yl}-2-phenyl-acetamide

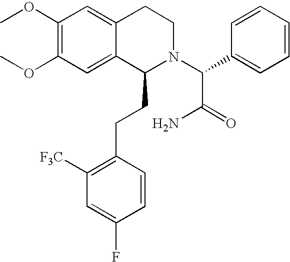

prepared by reaction of toluene-4-sulfonic acid carbamoyl-phenyl-methyl ester with (S)-1-[2-(4-fluoro-2-trifluoromethyl-phenyl)-ethyl]-6,7-dimethoxy-1,2,3,4-tetrahydro-isoquinoline.

LC-MS: rt=0.88 min, 517 (M+1, ES+).

### EXAMPLE 29

### (R)-2-{(S)-1-[2-(5-Fluoro-2-trifluoromethyl-phenyl)-ethyl]-6,7-dimethoxy-3,4-dihydro-1H-isoquinolin-2-yl}-2-phenyl-acetamide

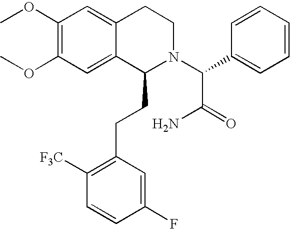

prepared by reaction of toluene-4-sulfonic acid carbamoyl-phenyl-methyl ester with (S)-1-[2-(5-fluoro-2-trifluoromethyl-phenyl)-ethyl]-6,7-dimethoxy-1,2,3,4-tetrahydro-isoquinoline.

LC-MS: rt=0.87 min, 517 (M+1, ES+).

### EXAMPLE 30

### (R)-2-{(S)-1-[2-(2-Fluoro-5-trifluoromethyl-phenyl)-ethyl]-6,7-dimethoxy-3,4-dihydro-1H-isoquinolin-2-yl}-2-phenyl-acetamide

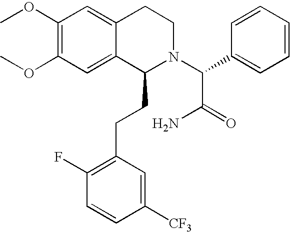

prepared by reaction of toluene-4-sulfonic acid carbamoyl-phenyl-methyl ester with (S)-1-[2-(2-fluoro-5-trifluoromethyl-phenyl)-ethyl]-6,7-dimethoxy-1,2,3,4-tetrahydro-isoquinoline.

LC-MS: rt=0.88 min, 517 (M+1, ES+).

### EXAMPLE 31

### (R)-2-{(S)-1-[2-(3-Fluoro-4-trifluoromethyl-phenyl)-ethyl]-6,7-dimethoxy-3,4-dihydro-1H-isoquinolin-2-yl}-2-phenyl-acetamide

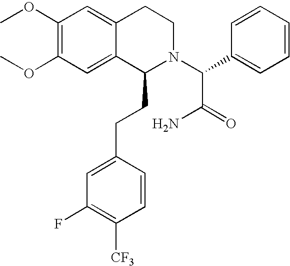

prepared by reaction of toluene-4-sulfonic acid carbamoyl-phenyl-methyl ester with (S)-1-[2-(3-fluoro-4-trifluoromethyl-phenyl)-ethyl]-6,7-dimethoxy-1,2,3,4-tetrahydro-isoquinoline.

LC-MS: rt=0.88 min, 517 (M+1, ES+).

### EXAMPLE 32

### (R)-2-{(S)-1-[2-(4-Fluoro-3-methyl-phenyl)-ethyl]-6,7-dimethoxy-3,4-dihydro-1H-isoquinolin-2-yl}-2-phenyl-acetamide

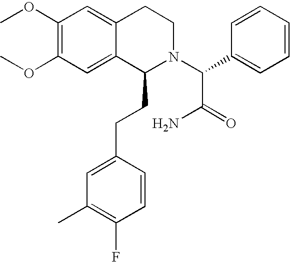

prepared by reaction of toluene-4-sulfonic acid carbamoyl-phenyl-methyl ester with (S)-1-[2-(4-fluoro-3-methyl-phenyl)-ethyl]-6,7-dimethoxy-1,2,3,4-tetrahydro-isoquinoline.

LC-MS: rt=0.85 min, 463 (M+1, ES+).

### EXAMPLE 33

### (R)-2-{(S)-6,7-Dimethoxy-1-[2-(2,3,5-trifluoro-phenyl)-ethyl]-3,4-dihydro-1H-isoquinolin-2-yl}-2-phenyl-acetamide

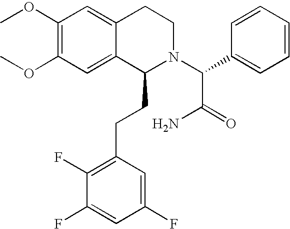

prepared by reaction of toluene-4-sulfonic acid carbamoyl-phenyl-methyl ester with (S)-6,7-dimethoxy-1-[2-(2,3,5-trifluoro-phenyl)-ethyl]-1,2,3,4-tetrahydro-isoquinoline.

LC-MS: rt=0.84 min, 485 (M+1, ES+).

### EXAMPLE 34

### (R)-2-{(S)-1-[2-(4-Fluoro-3-trifluoromethyl-phenyl)-ethyl]-6,7-dimethoxy-3,4-dihydro-1H-isoquinolin-2-yl}-2-phenyl-acetamide

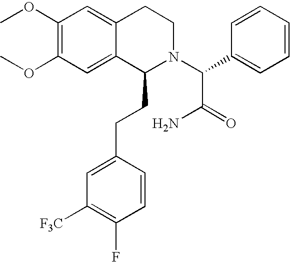

prepared by reaction of toluene-4-sulfonic acid carbamoyl-phenyl-methyl ester with (S)-1-[2-(4-fluoro-3-trifluoromethyl-phenyl)-ethyl]-6,7-dimethoxy-1,2,3,4-tetrahydro-isoquinoline.

LC-MS: rt=0.88 min, 517 (M+1, ES+).

### EXAMPLE 35

### (R)-2-{(S)-1-[2-(3-Fluoro-5-trifluoromethyl-phenyl)-ethyl]-6,7-dimethoxy-3,4-dihydro-1H-isoquinolin-2-yl}-2-phenyl-acetamide

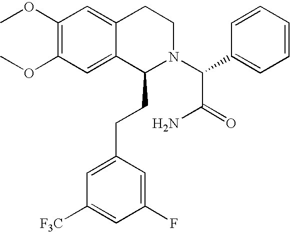

prepared by reaction of toluene-4-sulfonic acid carbamoyl-phenyl-methyl ester with (S)-1-[2-(3-fluoro-5-trifluoromethyl-phenyl)-ethyl]-6,7-dimethoxy-1,2,3,4-tetrahydro-isoquinoline.

LC-MS: rt=0.88 min, 517 (M+1, ES+).

### EXAMPLE 36

### (R)-2-{(S)-1-[2-(5-Chloro-2-trifluoromethyl-phenyl)-ethyl]-6,7-dimethoxy-3,4-dihydro-1H-isoquinolin-2-yl}-2-phenyl-acetamide

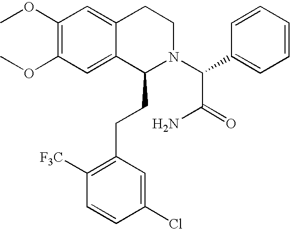

prepared by reaction of toluene-4-sulfonic acid carbamoyl-phenyl-methyl ester with (S)-1-[2-(5-chloro-2-trifluoromethyl-phenyl)-ethyl]-6,7-dimethoxy-1,2,3,4-tetrahydro-isoquinoline.

LC-MS: rt=0.90 min, 533 (M+1, ES+).

### EXAMPLE 37

### (R)-2-{(S)-1-[2-(2-Fluoro-3-trifluoromethyl-phenyl)-ethyl]-6,7-dimethoxy-3,4-dihydro-1H-isoquinolin-2-yl}-2-phenyl-acetamide

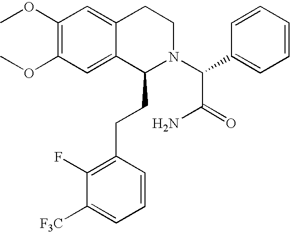

prepared by reaction of toluene-4-sulfonic acid carbamoyl-phenyl-methyl ester with (S)-1-[2-(2-fluoro-3-trifluoromethyl-phenyl)-ethyl]-6,7-dimethoxy-1,2,3,4-tetrahydro-isoquinoline.

LC-MS: rt=0.88 min, 517 (M+1, ES+).

### EXAMPLE 38

### (R)-2-{(S)-1-[2-(2-Fluoro-4-trifluoromethyl-phenyl)-ethyl]-6,7-dimethoxy-3,4-dihydro-1H-isoquinolin-2-yl}-2-phenyl-acetamide

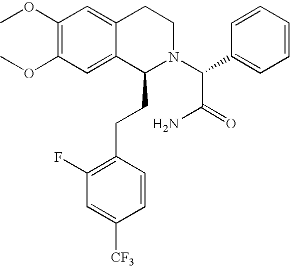

prepared by reaction of toluene-4-sulfonic acid carbamoyl-phenyl-methyl ester with (S)-1-[2-(2-fluoro-4-trifluoromethyl-phenyl)-ethyl]-6,7-dimethoxy-1,2,3,4-tetrahydro-isoquinoline.

LC-MS: rt=0.88 min, 517 (M+1, ES+).

### EXAMPLE 39

### (R)-2-{(S)-1-[2-(2-Difluoromethoxy-phenyl)-ethyl]-6,7-dimethoxy-3,4-dihydro-1H-isoquinolin-2-yl}-2-phenyl-acetamide

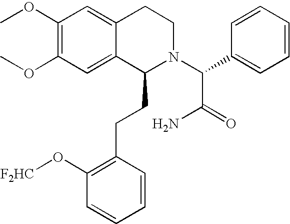

prepared by reaction of toluene-4-sulfonic acid carbamoyl-phenyl-methyl ester with (S)-1-[2-(2-difluoromethoxy-phenyl)-ethyl]-6,7-dimethoxy-1,2,3,4-tetrahydro-isoquinoline.

LC-MS: rt=0.84 min, 497 (M+1, ES+).

### EXAMPLE 40

### (R)-2-{(S)-1-[2-(3-Fluoro-2-trifluoromethyl-phenyl)-ethyl]-6,7-dimethoxy-3,4-dihydro-1H-isoquinolin-2-yl}-2-phenyl-acetamide

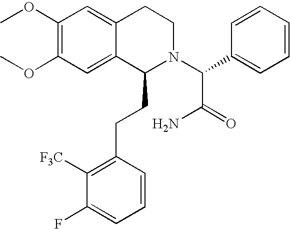

prepared by reaction of toluene-4-sulfonic acid carbamoyl-phenyl-methyl ester with (S)-1-[2-(3-fluoro-2-trifluoromethyl-phenyl)-ethyl]-6,7-dimethoxy-1,2,3,4-tetrahydro-isoquinoline.

LC-MS: rt=0.86 min, 517 (M+1, ES+).

### EXAMPLE 41

### (R)-2-{(S)-1-[2-(2-Chloro-3-trifluoromethyl-phenyl)-ethyl]-6,7-dimethoxy-3,4-dihydro-1H-isoquinolin-2-yl}-2-phenyl-acetamide

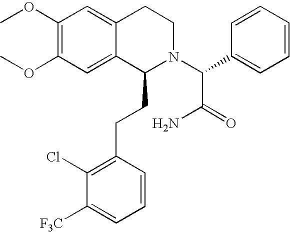

prepared by reaction of toluene-4-sulfonic acid carbamoyl-phenyl-methyl ester with (S)-1-[2-(2-chloro-3-trifluoromethyl-phenyl)-ethyl]-6,7-dimethoxy-1,2,3,4-tetrahydro-isoquinoline.

LC-MS: rt=0.89 min, 533 (M+1, ES+).

### EXAMPLE 42

### (R)-2-{(S)-1-[2-(2-Fluoro-6-trifluoromethyl-phenyl)-ethyl]-6,7-dimethoxy-3,4-dihydro-1H-isoquinolin-2-yl}-2-phenyl-acetamide

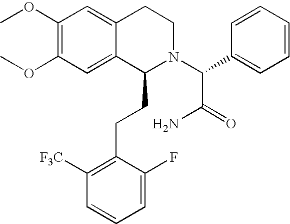

prepared by reaction of toluene-4-sulfonic acid carbamoyl-phenyl-methyl ester with (S)-1-[2-(2-fluoro-6-trifluoromethyl-phenyl)-ethyl]-6,7-dimethoxy-1,2,3,4-tetrahydro-isoquinoline.

LC-MS: rt=0.86 min, 517 (M+1, ES+).

### EXAMPLE 43

### (R)-2-{(S)-1-[2-(4-Chloro-3-trifluoromethyl-phenyl)-ethyl]-6,7-dimethoxy-3,4-dihydro-1H-isoquinolin-2-yl}-2-phenyl-acetamide

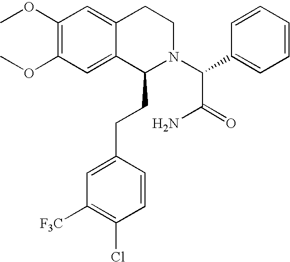

prepared by reaction of toluene-4-sulfonic acid carbamoyl-phenyl-methyl ester with (S)-1-[2-(4-chloro-3-trifluoromethyl-phenyl)-ethyl]-6,7-dimethoxy-1,2,3,4-tetrahydro-isoquinoline.

LC-MS: rt=0.90 min, 533 (M+1, ES+).

### EXAMPLE 44

### (R)-2-{(S)-1-[2-(2,3-Difluoro-4-methyl-phenyl)-ethyl]-6,7-dimethoxy-3,4-dihydro-1H-isoquinolin-2-yl}-2-phenyl-acetamide

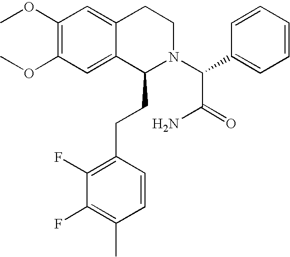

prepared by reaction of toluene-4-sulfonic acid carbamoyl-phenyl-methyl ester with (S)-1-[2-(2,3-difluoro-4-methyl-phenyl)-ethyl]-6,7-dimethoxy-1,2,3,4-tetrahydro-isoquinoline.

LC-MS: rt=0.86 min, 481 (M+1, ES+).

### EXAMPLE 45

### (R)-2-{(S)-1-[2-(4-Difluoromethoxy-phenyl)-ethyl]-6,7-dimethoxy-3,4-dihydro-1H-isoquinolin-2-yl}-2-phenyl-acetamide

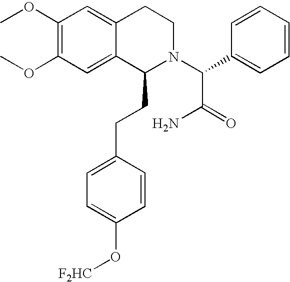

prepared by reaction of toluene-4-sulfonic acid carbamoyl-phenyl-methyl ester with (S)-1-[2-(4-difluoromethoxy-phenyl)-ethyl]-6,7-dimethoxy-1,2,3,4-tetrahydro-isoquinoline.

LC-MS: rt=0.84 min, 497 (M+1, ES+).

### EXAMPLE 46

### (R)-2-{(S)-1-[2-(3,4-Dimethyl-phenyl)-ethyl]-6,7-dimethoxy-3,4-dihydro-H-isoquinolin-2-yl}-2-phenyl-acetamide

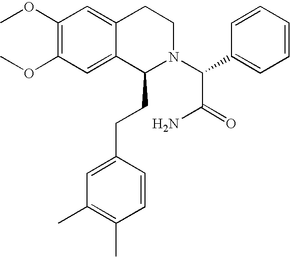

prepared by reaction of toluene-4-sulfonic acid carbamoyl-phenyl-methyl ester with (S)-1-[2-(3,4-dimethyl-phenyl)-ethyl]-6,7-dimethoxy-1,2,3,4-tetrahydro-isoquinoline.

LC-MS: rt=0.87 min, 459 (M+1, ES+).

### EXAMPLE 47

### (R)-2-{(S)-1-[2-(3-Chloro-4-methyl-phenyl)-ethyl]-6,7-dimethoxy-3,4-dihydro-1H-isoquinolin-2-yl}-2-phenyl-acetamide

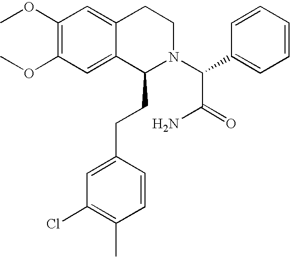

prepared by reaction of toluene-4-sulfonic acid carbamoyl-phenyl-methyl ester with (S)-1-[2-(3-chloro-4-methyl-phenyl)-ethyl]-6,7-dimethoxy-1,2,3,4-tetrahydro-isoquinoline.

LC-MS: rt=0.88 min, 479 (M+1, ES+).

### EXAMPLE 48

### (R)-2-{(S)-1-[2-(2-Chloro-6-fluoro-phenyl)-ethyl]-6,7-dimethoxy-3,4-dihydro-1H-isoquinolin-2-yl}-2-phenyl-acetamide

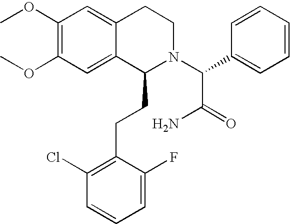

prepared by reaction of toluene-4-sulfonic acid carbamoyl-phenyl-methyl ester with (S)-1-[2-(2-chloro-6-fluoro-phenyl)-ethyl]-6,7-dimethoxy-1,2,3,4-tetrahydro-isoquinoline.

LC-MS: rt=0.85 min, 483 (M+1, ES+).

### EXAMPLE 49

### (R)-2-[(S)-6,7-Dimethoxy-1-(2-o-tolyl-ethyl)-3,4-dihydro-1H-isoquinolin-2-yl]-2-phenyl-acetamide

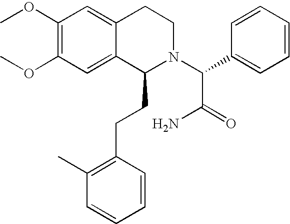

prepared by reaction of toluene-4-sulfonic acid carbamoyl-phenyl-methyl ester with (S)-6,7-dimethoxy-1-(2-o-tolyl-ethyl)-1,2,3,4-tetrahydro-isoquinoline.

LC-MS: rt=0.84 min, 445 (M+1, ES+).

### EXAMPLE 50

### (R)-2-[(S)-6,7-Dimethoxy-1-(2-m-tolyl-ethyl)-3,4-dihydro-1H-isoquinolin-2-yl]-2-phenyl-acetamide

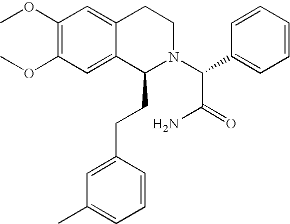

prepared by reaction of toluene-4-sulfonic acid carbamoyl-phenyl-methyl ester with (S)-6,7-dimethoxy-1-(2-m-tolyl-ethyl)-1,2,3,4-tetrahydro-isoquinoline.

LC-MS: rt=0.84 min, 445 (M+1, ES+).

### EXAMPLE 51

### (R)-2-[(S)-6,7-Dimethoxy-1-(2-p-tolyl-ethyl)-3,4-dihydro-1H-isoquinolin-2-yl]-2-phenyl-acetamide

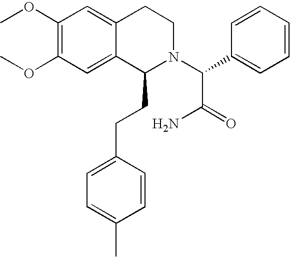

prepared by reaction of toluene-4-sulfonic acid carbamoyl-phenyl-methyl ester with (S)-6,7-dimethoxy-1-(2-p-tolyl-ethyl)-1,2,3,4-tetrahydro-isoquinoline.

LC-MS: rt=0.84 min, 445 (M+1, ES+).

### EXAMPLE 52

### (R)-2-{(S)-1-[2-(2-Fluoro-phenyl)-ethyl]-6,7-dimethoxy-3,4-dihydro-1H-isoquinolin-2-yl}-2-phenyl-acetamide

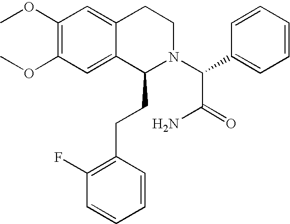

prepared by reaction of toluene-4-sulfonic acid carbamoyl-phenyl-methyl ester with (S)-1-[2-(2-fluoro-phenyl)-ethyl]-6,7-dimethoxy-1,2,3,4-tetrahydro-isoquinoline.

LC-MS: rt=0.82 min, 449 (M+1, ES+).

### EXAMPLE 53

### (R)-2-{(S)-1-[2-(3-Fluoro-phenyl)-ethyl]-6,7-dimethoxy-3,4-dihydro-1H-isoquinolin-2-yl}-2-phenyl-acetamide

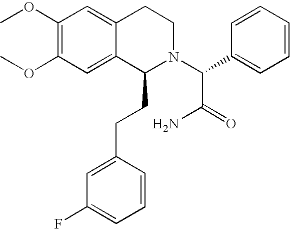

prepared by reaction of toluene-4-sulfonic acid carbamoyl-phenyl-methyl ester with (S)-1-[2-(3-fluoro-phenyl)-ethyl]-6,7-dimethoxy-1,2,3,4-tetrahydro-isoquinoline.

LC-MS: rt=0.81 min, 449 (M+1, ES+).

### EXAMPLE 54

### (R)-2-{(S)-1-[2-(2,6-Dichloro-phenyl)-ethyl]-6,7-dimethoxy-3,4-dihydro-1H-isoquinolin-2-yl}-2-phenyl-acetamide

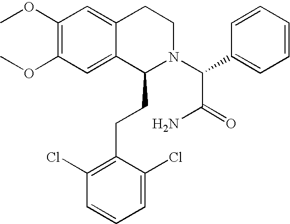

prepared by reaction of toluene-4-sulfonic acid carbamoyl-phenyl-methyl ester with (S)-1-[2-(2,6-dichloro-phenyl)-ethyl]-6,7-dimethoxy-1,2,3,4-tetrahydro-isoquinoline.

LC-MS: rt 0.87 min, 499 (M+1, ES+).

### EXAMPLE 55

### (R)-2-{(S)-1-[2-(3,4-Dichloro-phenyl)-ethyl]-6,7-dimethoxy-3,4-dihydro-1H-isoquinolin-2-yl}-2-phenyl-acetamide

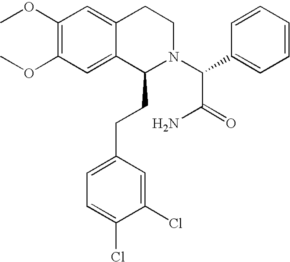

prepared by reaction of toluene-4-sulfonic acid carbamoyl-phenyl-methyl ester with (S)-1-[2-(3,4-dichloro-phenyl)-ethyl]-6,7-dimethoxy-1,2,3,4-tetrahydro-isoquinoline.

LC-MS: rt=0.89 min, 499 (M+1, ES+).

### EXAMPLE 56

### (R)-2-{(S)-1-[2-(3,5-Dimethyl)-ethyl]-6,7-dimethoxy-3,4-dihydro-1H-isoquinolin-2-yl}-2-phenyl-acetamide

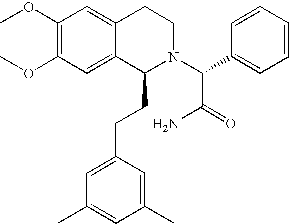

prepared by reaction of toluene-4-sulfonic acid carbamoyl-phenyl-methyl ester with (S)-1-[2-(3,5-dimethyl-phenyl)-ethyl]-6,7-dimethoxy-1,2,3,4-tetrahydro-isoquinoline.

LC-MS: rt=0.87 min, 459 (M+1, ES+).

### EXAMPLE 57

### (R)-2-{(S)-6,7-Dimethoxy-1-[2-(2-trifluoromethyl-phenyl)-ethyl]-3,4-dihydro-1H-isoquinolin-2-yl}-2-phenyl-acetamide

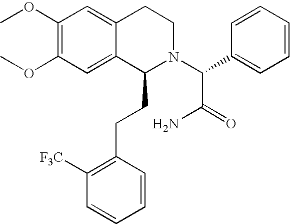

prepared by reaction of toluene-4-sulfonic acid carbamoyl-phenyl-methyl ester with (S)-6,7-dimethoxy-1-[2-(2-trifluoromethyl-phenyl)-ethyl]-1,2,3,4-tetrahydro-isoquinoline.

LC-MS: rt=0.86 min, 499 (M+1, ES+).

### EXAMPLE 58

### (R)-2-{(S)-1-[2-(2,4-Difluoro-phenyl)-ethyl]-6,7-dimethoxy-3,4-dihydro-1H-isoquinolin-2-yl}-2-phenyl-acetamide

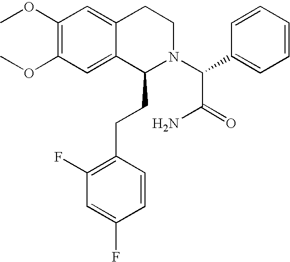

prepared by reaction of toluene-4-sulfonic acid carbamoyl-phenyl-methyl ester with (S)-1-[2-(2,4-difluoro-phenyl)-ethyl]-6,7-dimethoxy-1,2,3,4-tetrahydro-isoquinoline.

LC-MS: rt=0.83 min, 467 (M+1, ES+).

### EXAMPLE 59

### (R)-2-{(S)-6,7-Dimethoxy-1-[2-(2,3,6-trifluoro-phenyl)-ethyl]-3,4-dihydro-1H-isoquinolin-2-yl}-2-phenyl-acetamide

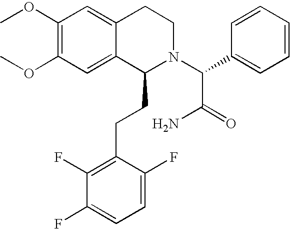

prepared by reaction of toluene-4-sulfonic acid carbamoyl-phenyl-methyl ester with (S)-6,7-dimethoxy-1-[2-(2,3,6-trifluoro-phenyl)-ethyl]-1,2,3,4-tetrahydro-isoquinoline.

LC-MS: rt=0.83 min, 485 (M+1, ES+).

### EXAMPLE 60

### (R)-2-{(S)-6,7-Dimethoxy-1-[2-(4-trifluoromethoxy-phenyl)-ethyl]-3,4-dihydro-1H-isoquinolin-2-yl}-2-phenyl-acetamide

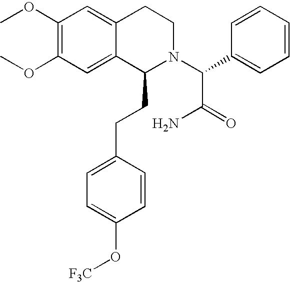

prepared by reaction of toluene-4-sulfonic acid carbamoyl-phenyl-methyl ester with (S)-6,7-dimethoxy-1-[2-(4-trifluoromethoxy-phenyl)-ethyl]-1,2,3,4-tetrahydro-isoquinoline.

LC-MS: rt=0.88 min, 515 (M+1, ES+).

### EXAMPLE 61

### (R)-2-{(S)-1-[2-(2-Fluoro-3-methyl-phenyl)-ethyl]-6,7-dimethoxy-3,4-dihydro-1H-isoquinolin-2-yl}-2-phenyl-acetamide

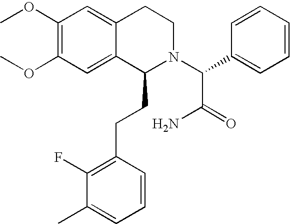

prepared by reaction of toluene-4-sulfonic acid carbamoyl-phenyl-methyl ester with (S)-1-[2-(2-fluoro-3-methyl-phenyl)-ethyl]-6,7-dimethoxy-1,2,3,4-tetrahydro-isoquinoline.

LC-MS: rt=0.85 min, 463 (M+1, ES+).

### EXAMPLE 62

### (R)-2-{(S)-1-[2-(4-Chloro-phenyl)-ethyl]-6,7-dimethoxy-3,4-dihydro-1H-isoquinolin-2-yl}-2-phenyl-acetamide

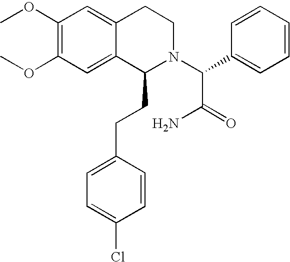

prepared by reaction of toluene-4-sulfonic acid carbamoyl-phenyl-methyl ester with (S)-1-[2-(4-chloro-phenyl)-ethyl]-6,7-dimethoxy-1,2,3,4-tetrahydro-isoquinoline.

LC-MS: rt=0.85 min, 465 (M+1, ES+).

### EXAMPLE 63

### (R)-2-{(S)-1-[2-(3-Chloro-phenyl)-ethyl]-6,7-dimethoxy-3,4-dihydro-1H-isoquinolin-2-yl}-2-phenyl-acetamide

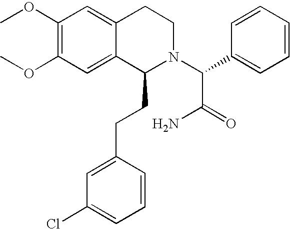

prepared by reaction of toluene-4-sulfonic acid carbamoyl-phenyl-methyl ester with (S)-1-[2-(3-chloro-phenyl)-ethyl]-6,7-dimethoxy-1,2,3,4-tetrahydro-isoquinoline.

LC-MS: rt=0.85 min, 465 (M+1, ES+).

### EXAMPLE 64

### (R)-2-{(S)-6,7-Dimethoxy-1-[2-(3-trifluoromethoxy-phenyl)-ethyl]-3,4-dihydro-1H-isoquinolin-2-yl}-2-phenyl-acetamide

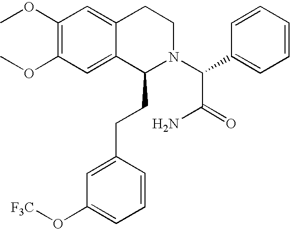

prepared by reaction of toluene-4-sulfonic acid carbamoyl-phenyl-methyl ester with (S)-6,7-dimethoxy-1-[2-(3-trifluoromethoxy-phenyl)-ethyl]-1,2,3,4-tetrahydro-isoquinoline.

LC-MS: rt=0.89 min, 515 (M+1, ES+).

### EXAMPLE 65

### (R)-2-{(S)-6,7-Dimethoxy-1-[2-(3,4,5-trifluoro-phenyl)-ethyl]-3,4-dihydro-1H-isoquinolin-2-yl}-2-phenyl-acetamide

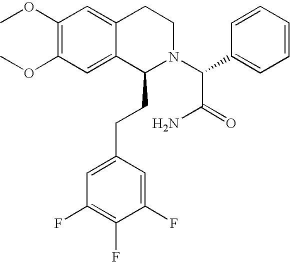

prepared by reaction of toluene-4-sulfonic acid carbamoyl-phenyl-methyl ester with (S)-6,7-dimethoxy-1-[2-(3,4,5-trifluoro-phenyl)-ethyl]-1,2,3,4-tetrahydro-isoquinoline.

LC-MS: rt=0.85 min, 485 (M+1, ES+).

### EXAMPLE 66

### (R)-2-{(S)-6,7-Dimethoxy-1-[2-(3-trifluoromethyl-phenyl)-ethyl]-3,4-dihydro-1H-isoquinolin-2-yl}-2-phenyl-acetamide

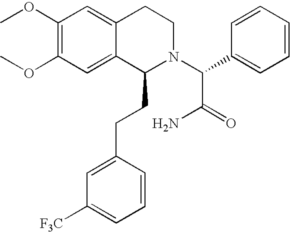

prepared by reaction of toluene-4-sulfonic acid carbamoyl-phenyl-methyl ester with (S)-6,7-dimethoxy-1-[2-(3-trifluoromethyl-phenyl)-ethyl]-1,2,3,4-tetrahydro-isoquinoline.

LC-MS: rt=0.87 min, 499 (M+1, ES+).

### EXAMPLE 67

### (R)-2-{(S)-6,7-Dimethoxy-1-[2-(2,3,4-trifluoro-phenyl)-ethyl]-3,4-dihydro-1H-isoquinolin-2-yl}-2-phenyl-acetamide

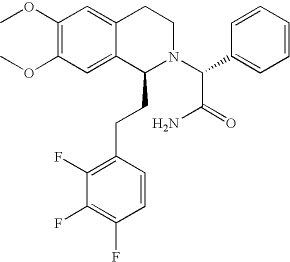

prepared by reaction of toluene-4-sulfonic acid carbamoyl-phenyl-methyl ester with (S)-6,7-dimethoxy-1-[2-(2,3,4-trifluoro-phenyl)-ethyl]-1,2,3,4-tetrahydro-isoquinoline.

LC-MS: rt=0.85 min, 485 (M+1, ES+).

### EXAMPLE 68

### (R)-2-{(S)-1-[2-(4-Bromo-2-fluoro-phenyl)-ethyl]-6,7-dimethoxy-3,4-dihydro-1H-isoquinolin-2-yl}-2-phenyl-acetamide

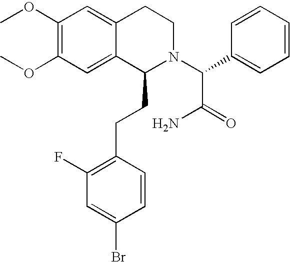

prepared by reaction of toluene-4-sulfonic acid carbamoyl-phenyl-methyl ester with (S)-1-[2-(4-bromo-2-fluoro-phenyl)-ethyl]-6,7-dimethoxy-1,2,3,4-tetrahydro-isoquinoline.

LC-MS: rt=0.87 min, 527 (M+1, ES+).

### EXAMPLE 69

### (R)-2-{(S)-1-[2-(2,6-Difluoro-phenyl)-ethyl]-6,7-dimethoxy-3,4-dihydro-1H-isoquinolin-2-yl}-2-phenyl-acetamide

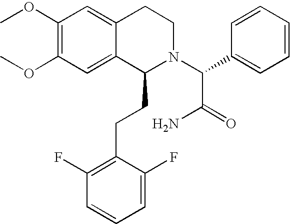

prepared by reaction of toluene-4-sulfonic acid carbamoyl-phenyl-methyl ester with (S)-1-[2-(2,6-difluoro-phenyl)-ethyl]-6,7-dimethoxy-1,2,3,4-tetrahydro-isoquinoline.

LC-MS: rt=0.82 min, 467 (M+1, ES+).

### EXAMPLE 70

### (R)-2-{(S)-6,7-Dimethoxy-1-[2-(2-trifluoromethoxy-phenyl)-ethyl]-3,4-dihydro-1H-isoquinolin-2-yl}-2-phenyl-acetamide

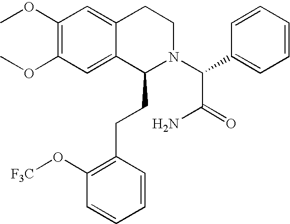

prepared by reaction of toluene-4-sulfonic acid carbamoyl-phenyl-methyl ester with (S)-6,7-dimethoxy-1-[2-(2-trifluoromethoxy-phenyl)-ethyl]-1,2,3,4-tetrahydro-isoquinoline.

LC-MS: rt=0.87 min, 515 (M+1, ES+).

### EXAMPLE 71

### (R)-2-{(S)-1-[2-(2,4-Dichloro-phenyl)-ethyl]-6,7-dimethoxy-3,4-dihydro-1H-isoquinolin-2-yl}-2-phenyl-acetamide

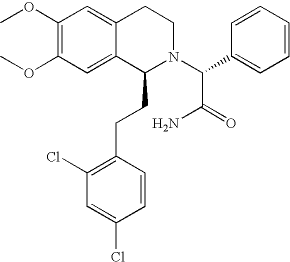

prepared by reaction of toluene-4-sulfonic acid carbamoyl-phenyl-methyl ester with (S)-1-[2-(2,4-dichloro-phenyl)-ethyl]-6,7-dimethoxy-1,2,3,4-tetrahydro-isoquinoline.

LC-MS: rt=0.89 min, 499 (M+1, ES+).

### EXAMPLE 72

### (R)-2-{(S)-6,7-Dimethoxy-1-[2-(2,4,5-trifluoro-phenyl)-ethyl]-3,4-dihydro-1H-isoquinolin-2-yl}-2-phenyl-acetamide

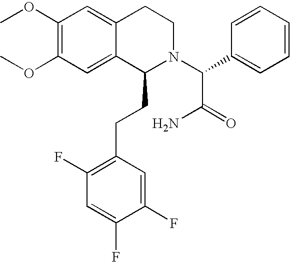

prepared by reaction of toluene-4-sulfonic acid carbamoyl-phenyl-methyl ester with (S)-6,7-dimethoxy-1-[2-(2,4,5-trifluoro-phenyl)-ethyl]-1,2,3,4-tetrahydro-isoquinoline.

LC-MS: rt=0.84 min, 485 (M+1, ES+).

### EXAMPLE 73

### (R)-2-{(S)-1-[2-(3-Bromo-phenyl)-ethyl]-6,7-dimethoxy-3,4-dihydro-1H-isoquinolin-2-yl}-2-phenyl-acetamide

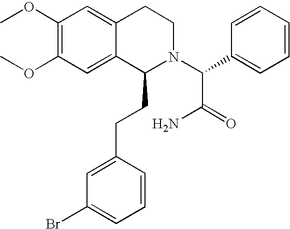

prepared by reaction of toluene-4-sulfonic acid carbamoyl-phenyl-methyl ester with (S)-1-[2-(3-bromo-phenyl)-ethyl]-6,7-dimethoxy-1,2,3,4-tetrahydro-isoquinoline.

LC-MS: rt=0.86 min, 509 (M+1, ES+).

### EXAMPLE 74

### (R)-2-{(S)-1-[2-(4-tert-Butyl-phenyl)-ethyl]-6,7-dimethoxy-3,4-dihydro-1H-isoquinolin-2-yl}-2-phenyl-acetamide

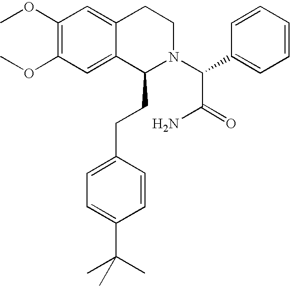

prepared by reaction of toluene-4-sulfonic acid carbamoyl-phenyl-methyl ester with (S)-1-[2-(4-tert-butyl-phenyl)-ethyl]-6,7-dimethoxy-1,2,3,4-tetrahydro-isoquinoline.

LC-MS: rt=0.92 min, 487 (M+1, ES+).

### EXAMPLE 75

### (R)-2-{(S)-1-[2-(2-Bromo-phenyl)-ethyl]-6,7-dimethoxy-3,4-dihydro-1H-isoquinolin-2-yl}-2-phenyl-acetamide

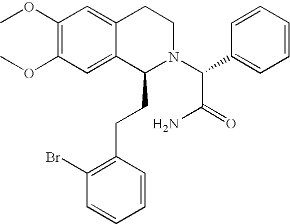

prepared by reaction of toluene-4-sulfonic acid carbamoyl-phenyl-methyl ester with (S)-1-[2-(2-bromo-phenyl)-ethyl]-6,7-dimethoxy-1,2,3,4-tetrahydro-isoquinoline.

LC-MS: rt=0.85 min, 509 (M+1, ES+).

### EXAMPLE 76

### (R)-2-{(S)-1-[2-(4-Bromo-phenyl)-ethyl]-6,7-dimethoxy-3,4-dihydro-1H-isoquinolin-2-yl}-2-phenyl-acetamide

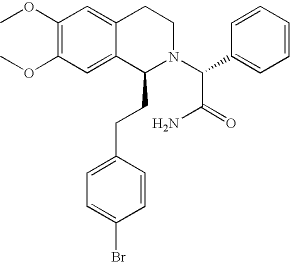

prepared by reaction of toluene-4-sulfonic acid carbamoyl-phenyl-methyl ester with (S)-1-[2-(4-bromo-phenyl)-ethyl]-6,7-dimethoxy-1,2,3,4-tetrahydro-isoquinoline.

LC-MS: rt=0.86 min, 509 (M+1, ES+).

G.5 Synthesis of α-Substituted Glycinamides via Alkylation of Enantiomerically Enriched 1,2,3,4-tetrahydroisoquinolines with (S)-Toluene-4-sulfonic Acid carbamoyl-phenyl-methyl Ester (General Procedure):

A solution of the respective enantiomerically enriched 1,2,3,4-tetrahydro-isoquinoline derivative (19.7 mmol), DIPEA (27.6 mmol) and (S)-toluene-4-sulfonic acid carbamoyl-phenyl-methyl ester (23.6 mmol) in 2-butanone is stirred under reflux for 2 d. Silica gel (106 g) and DCM (100 mL) are added, the solvents are removed in vacuo and the residue is purified by flash chromatography (gradient: ethyl acetate/heptane 1/2 to 4/1) to give the following acetamide derivatives:

### EXAMPLE 77

### (R)-2-{(S)-6,7-Dimethoxy-1-[2-(4-trifluoromethyl-phenyl)-ethyl]-3,4-dihydro-1H-isoquinolin-2-yl}-2-phenyl-acetamide

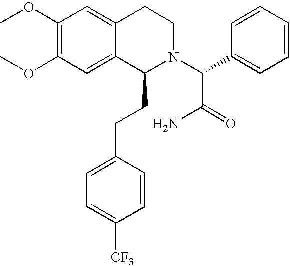

prepared by reaction of (S)-toluene-4-sulfonic acid carbamoyl-phenyl-methyl ester with (S)-6,7-dimethoxy-1-[2-(4-trifluoromethyl-phenyl)-ethyl]-1,2,3,4-tetrahydro-isoquinoline.

LC-MS: rt=0.86 min, 499 (M+1, ES+).

1. Novel compounds of formula (I) 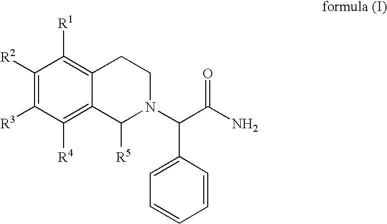
wherein
R¹, R², R³, R⁴ independently represent hydrogen; cyano; halogen; hydroxyl; C1-C4 alkyl; C2-C4 alkenyl; C1-C4 alkoxy; C2-C3 alkenyloxy; trifluoromethoxy; C3-C6 cycloalkyloxy or R¹ and R² together as well as R² and R³ together or R³ an R⁴ together may form with the phenyl ring, to which they are attached, a five, six or seven-membered ring containing one or two oxygen atoms;
R⁵ represents an unsubstituted or a mono-, di- or trisubstituted phenyl-ethyl group, substituted independently with C1-C4 alkyl, C1-C4 alkoxy, C2-C4 alkenyl, trifluoromethyl, triflourmethoxy, diflourmethoxy or halogen;
and pharmaceutically acceptable salts thereof.

2. Compounds of formula I, wherein
R¹ an R⁴ represent hydrogen;
R² and R³ represent C1-C4 alkoxy and
R⁵ represents a 2-phenylethyl group;
and pharmaceutically acceptable acid addition salts thereof.

3. Compounds of formula I, wherein
R¹ and R⁴ represent hydrogen;
R² and R³ represent C1-C4 alkoxy and
R⁵ represents a 2-phenylethyl group in which the phenyl group carries one, two or three substituents, each independently selected from C1-C4 alkyl or halogen;
and pharmaceutically acceptable acid addition salts thereof.

4. Compounds of formula I, wherein
R¹ and R⁴ represent hydrogen;
R² and R³ represent C1-C4 alkoxy and
R⁵ represents a 2-phenylethyl group in which the phenyl group carries one or two substituents, each independently selected from C1-C4 alkoxy or halogen;
and pharmaceutically acceptable acid addition salts thereof.

5. Compounds of formula I, wherein
R¹ and R⁴ represent hydrogen;
R² and R³ represent C1-C4 alkoxy and
R⁵ represents a 2-phenylethyl group in which the phenyl group carries one or two substituents, each independently selected from trifluoromethyl or halogen;
and pharmaceutically acceptable acid addition salts thereof.

6. Compounds of formula I, wherein
R¹ and R⁴ represent hydrogen;
R² and R³ represent C1-C4 alkoxy and
R⁵ represents a 2-phenylethyl group in which the phenyl group carries one or two substituents, each independently selected from difluoromethoxy or halogen;
and pharmaceutically acceptable acid addition salts thereof.

7. Compounds of formula I, wherein
R¹ and R⁴ represent hydrogen;
R² and R³ represent C1-C4 alkoxy and
R⁵ represents a 2-phenylethyl group in which the phenyl group carries one or two substituents, each independently selected from trifluoromethoxy or halogen;
and pharmaceutically acceptable acid addition salts thereof.

8. Compounds according to claim 1 selected from the group consisting of:
2-{1-[2-(3,4-Difluoro-phenyl)-ethyl]-6,7-dimethoxy-3,4-dihydro-1H-isoquinolin-2-yl}-2-phenyl-acetamide;
2-{1-[2-(2,3-Difluoro-phenyl)-ethyl]-6,7-dimethoxy-3,4-dihydro-1H-isoquinolin-2-yl}-2-phenyl-acetamide;
2-{6,7-Dimethoxy-1-[2-(4-trifluoromethyl-phenyl)-ethyl]-3,4-dihydro-1H-isoquinolin-2-yl}-2-phenyl-acetamide;
2-{6,7-Dimethoxy-1-[2-(3-methoxy-phenyl)-ethyl]-3,4-dihydro-1H-isoquinolin-2-yl}-2-phenyl-acetamide;
2-{1-[2-(2,5-Difluoro-phenyl)-ethyl]-6,7-dimethoxy-3,4-dihydro-1H-isoquinolin-2-yl}-2-phenyl-acetamide;
2-{6,7-Dimethoxy-1-[2-(2-methoxy-phenyl)-ethyl]-3,4-dihydro-1H-isoquinolin-2-yl}-2-phenyl-acetamide;
2-{1-[2-(4-Fluoro-phenyl)-ethyl]-6,7-dimethoxy-3,4-dihydro-1H-isoquinolin-2-yl}-2-phenyl-acetamide;
2-{1-[2-(3,4-Dimethoxy-phenyl)-ethyl]-6,7-dimethoxy-3,4-dihydro-1H-isoquinolin-2-yl}-2-phenyl-acetamide;
(R)-2-{(S)-6,7-Dimethoxy-1-[2-(2-methyl-5-trifluoromethyl-phenyl)-ethyl]-3,4-dihydro-1H-isoquinolin-2-yl}-2-phenyl-acetamide;
(R)-2-{(S)-1-[2-(3-Chloro-2-fluoro-phenyl)-ethyl]-6,7-dimethoxy-3,4-dihydro-1H-isoquinolin-2-yl}-2-phenyl-acetamide;
(R)-2-{(S)-1-[2-(3-Fluoro-4-methyl-phenyl)-ethyl]-6,7-dimethoxy-3,4-dihydro-1H-isoquinolin-2-yl}-2-phenyl-acetamide;
(R)-2-{(S)-1-[2-(4-Fluoro-2-trifluoromethyl-phenyl)-ethyl]-6,7-dimethoxy-3,4-dihydro-1H-isoquinolin-2-yl}-2-phenyl-acetamide;
(R)-2-{(S)-1-[2-(5-Fluoro-2-trifluoromethyl-phenyl)-ethyl]-6,7-dimethoxy-3,4-dihydro-1H-isoquinolin-2-yl}-2-phenyl-acetamide;
(R)-2-{(S)-1-[2-(2-Fluoro-5-trifluoromethyl-phenyl)-ethyl]-6,7-dimethoxy-3,4-dihydro-1H-isoquinolin-2-yl}-2-phenyl-acetamide;
(R)-2-{(S)-1-[2-(3-Fluoro-4-trifluoromethyl-phenyl)-ethyl]-6,7-dimethoxy-3,4-dihydro-1H-isoquinolin-2-yl}-2-phenyl-acetamide;
(R)-2-{(S)-1-[2-(4-Fluoro-3-methyl-phenyl)-ethyl]-6,7-dimethoxy-3,4-dihydro-1H-isoquinolin-2-yl}-2-phenyl-acetamide;
(R)-2-{(S)-6,7-Dimethoxy-1-[2-(2,3,5-trifluoro-phenyl)-ethyl]-3,4-dihydro-1H-isoquinolin-2-yl}-2-phenyl-acetamide;
(R)-2-{(S)-1-[2-(4-Fluoro-3-trifluoromethyl-phenyl)-ethyl]-6,7-dimethoxy-3,4-dihydro-1H-isoquinolin-2-yl}-2-phenyl-acetamide;
(R)-2-{(S)-1-[2-(3-Fluoro-5-trifluoromethyl-phenyl)-ethyl]-6,7-dimethoxy-3,4-dihydro-1H-isoquinolin-2-yl}-2-phenyl-acetamide;
(R)-2-{(S)-1-[2-(5-Chloro-2-trifluoromethyl-phenyl)-ethyl]-6,7-dimethoxy-3,4-dihydro-1H-isoquinolin-2-yl}-2-phenyl-acetamide;
(R)-2-{(S)-1-[2-(2-Fluoro-3-trifluoromethyl-phenyl)-ethyl]-6,7-dimethoxy-3,4-dihydro-1H-isoquinolin-2-yl}-2-phenyl-acetamide;
(R)-2-{(S)-1-[2-(2-Fluoro-4-trifluoromethyl-phenyl)-ethyl]-6,7-dimethoxy-3,4-dihydro-1H-isoquinolin-2-yl}-2-phenyl-acetamide;
(R)-2-{(S)-1-[2-(2-Difluoromethoxy-phenyl)-ethyl]-6,7-dimethoxy-3,4-dihydro-1H-isoquinolin-2-yl}-2-phenyl-acetamide;
(R)-2-{(S)-1-[2-(3-Fluoro-2-trifluoromethyl-phenyl)-ethyl]-6,7-dimethoxy-3,4-dihydro-1H-isoquinolin-2-yl}-2-phenyl-acetamide;
(R)-2-{(S)-1-[2-(2-Chloro-3-trifluoromethyl-phenyl)-ethyl]-6,7-dimethoxy-3,4-dihydro-1H-isoquinolin-2-yl}-2-phenyl-acetamide;
(R)-2-{(S)-1-[2-(2-Fluoro-6-trifluoromethyl-phenyl)-ethyl]-6,7-dimethoxy-3,4-dihydro-1H-isoquinolin-2-yl}-2-phenyl-acetamide;
(R)-2-{(S)-1-[2-(4-Chloro-3-trifluoromethyl-phenyl)-ethyl]-6,7-dimethoxy-3,4-dihydro-1H-isoquinolin-2-yl}-2-phenyl-acetamide;
(R)-2-{(S)-1-[2-(2,3-Difluoro-4-methyl-phenyl)-ethyl]-6,7-dimethoxy-3,4-dihydro-1H-isoquinolin-2-yl}-2-phenyl-acetamide;
(R)-2-{(S)-1-[2-(4-Difluoromethoxy-phenyl)-ethyl]-6,7-dimethoxy-3,4-dihydro-1H-isoquinolin-2-yl}-2-phenyl-acetamide;
(R)-2-{(S)-1-[2-(3,4-Dimethyl-phenyl)-ethyl]-6,7-dimethoxy-3,4-dihydro-1H-isoquinolin-2-yl}-2-phenyl-acetamide;
(R)-2-{(S)-1-[2-(3-Chloro-4-methyl-phenyl)-ethyl]-6,7-dimethoxy-3,4-dihydro-1H-isoquinolin-2-yl}-2-phenyl-acetamide;
(R)-2-{(S)-1-[2-(2-Chloro-6-fluoro-phenyl)-ethyl]-6,7-dimethoxy-3,4-dihydro-1H-isoquinolin-2-yl}-2-phenyl-acetamide;
(R)-2-[(S)-6,7-Dimethoxy-1-(2-o-tolyl-ethyl)-3,4-dihydro-1H-isoquinolin-2-yl]-2-phenyl-acetamide;
(R)-2-[(S)-6,7-Dimethoxy-1-(2-m-tolyl-ethyl)-3,4-dihydro-1H-isoquinolin-2-yl]-2-phenyl-acetamide;
(R)-2-[(S)-6,7-Dimethoxy-1-(2-p-tolyl-ethyl)-3,4-dihydro-1H-isoquinolin-2-yl]-2-phenyl-acetamide;
(R)-2-{(S)-1-[2-(2-Fluoro-phenyl)-ethyl]-6,7-dimethoxy-3,4-dihydro-1H-isoquinolin-2-yl}-2-phenyl-acetamide;
(R)-2-{(S)-1-[2-(3-Fluoro-phenyl)-ethyl]-6,7-dimethoxy-3,4-dihydro-1H-isoquinolin-2-yl}-2-phenyl-acetamide;
(R)-2-{(S)-1-[2-(3,4-Dichloro-phenyl)-ethyl]-6,7-dimethoxy-3,4-dihydro-1H-isoquinolin-2-yl}-2-phenyl-acetamide;
(R)-2-{(S)-1-[2-(3,5-Dimethyl-phenyl)-ethyl]-6,7-dimethoxy-3,4-dihydro-1H-isoquinolin-2-yl}-2-phenyl-acetamide;
(R)-2-{(S)-6,7-Dimethoxy-1-[2-(2-trifluoromethyl-phenyl)-ethyl]-3,4-dihydro-1H-isoquinolin-2-yl}-2-phenyl-acetamide;
(R)-2-{(S)-1-[2-(2,4-Difluoro-phenyl)-ethyl]-6,7-dimethoxy-3,4-dihydro-1H-isoquinolin-2-yl}-2-phenyl-acetamide;
(R)-2-{(S)-6,7-Dimethoxy-1-[2-(2,3,6-trifluoro-phenyl)-ethyl]-3,4-dihydro-1H-isoquinolin-2-yl}-2-phenyl-acetamide;
(R)-2-{(S)-6,7-Dimethoxy-1-[2-(4-trifluoromethoxy-phenyl)-ethyl]-3,4-dihydro-1H-isoquinolin-2-yl}-2-phenyl-acetamide;
(R)-2-{(S)-1-[2-(2-Fluoro-3-methyl-phenyl)-ethyl]-6,7-dimethoxy-3,4-dihydro-1H-isoquinolin-2-yl}-2-phenyl-acetamide;
(R)-2-{(S)-1-[2-(4-Chloro-phenyl)-ethyl]-6,7-dimethoxy-3,4-dihydro-1H-isoquinolin-2-yl}-2-phenyl-acetamide;
(R)-2-{(S)-1-[2-(3-Chloro-phenyl)-ethyl]-6,7-dimethoxy-3,4-dihydro-1H-isoquinolin-2-yl}-2-phenyl-acetamide;
(R)-2-{(S)-6,7-Dimethoxy-1-[2-(3-trifluoromethoxy-phenyl)-ethyl]-3,4-dihydro-1H-isoquinolin-2-yl}-2-phenyl-acetamide;
(R)-2-{(S)-6,7-Dimethoxy-1-[2-(3,4,5-trifluoro-phenyl)-ethyl]-3,4-dihydro-1H-isoquinolin-2-yl}-2-phenyl-acetamide;
(R)-2-{(S)-6,7-Dimethoxy-1-[2-(3-trifluoromethyl-phenyl)-ethyl]-3,4-dihydro-1H-isoquinolin-2-yl}-2-phenyl-acetamide;
(R)-2-{(S)-6,7-Dimethoxy-1-[2-(2,3,4-trifluoro-phenyl)-ethyl]-3,4-dihydro-1H-isoquinolin-2-yl}-2-phenyl-acetamide;
(R)-2-{(S)-1-[2-(4-Bromo-2-fluoro-phenyl)-ethyl]-6,7-dimethoxy-3,4-dihydro-1H-isoquinolin-2-yl}-2-phenyl-acetamide;
(R)-2-{(S)-1-[2-(2,6-Difluoro-phenyl)-ethyl]-6,7-dimethoxy-3,4-dihydro-1H-isoquinolin-2-yl}-2-phenyl-acetamide;
(R)-2-{(S)-6,7-Dimethoxy-1-[2-(2-trifluoromethoxy-phenyl)-ethyl]-3,4-dihydro-1H-isoquinolin-2-yl}-2-phenyl-acetamide;
(R)-2-{(S)-1-[2-(2,4-Dichloro-phenyl)-ethyl]-6,7-dimethoxy-3,4-dihydro-1H-isoquinolin-2-yl}-2-phenyl-acetamide;
(R)-2-{(S)-6,7-Dimethoxy-1-[2-(2,4,5-trifluoro-phenyl)-ethyl]-3,4-dihydro-1H-isoquinolin-2-yl}-2-phenyl-acetamide;
(R)-2-{(S)-1-[2-(3-Bromo-phenyl)-ethyl]-6,7-dimethoxy-3,4-dihydro-1H-isoquinolin-2-yl}-2-phenyl-acetamide;
(R)-2-{(S)-1-[2-(2-Bromo-phenyl)-ethyl]-6,7-dimethoxy-3,4-dihydro-1H-isoquinolin-2-yl}-2-phenyl-acetamide;
(R)-2-{(S)-1-[2-(4-Bromo-phenyl)-ethyl]-6,7-dimethoxy-3,4-dihydro-1H-isoquinolin-2-yl}-2-phenyl-acetamide;
(R)-2-{(S)-6,7-Dimethoxy-1-[2-(4-trifluoromethyl-phenyl)-ethyl]-3,4-dihydro-1H-isoquinolin-2-yl}-2-phenyl-acetamide.

9. Pharmaceutical compositions for the treatment of disorders which are associated with the role of orexin, comprising one or more compounds of claim 1, or a pharmaceutically acceptable salt thereof, and usual carrier materials and adjuvants.

10. Pharmaceutical compositions for the treatment of eating and sleep disorders, comprising one or more compounds of claim 1, or a pharmaceutically acceptable salt thereof, and usual carrier materials and adjuvants.

11. The compounds of claim 1, or a pharmaceutically acceptable salt thereof, for use as medicaments for the treatment of disorders which are associated with a role of orexins.

12. Use of a 1,2,3,4-tetrahydroisoquinoline derivative according to claim 1, or a pharmaceutically acceptable salt thereof, in the preparation of a medicament for the prevention or treatment of diseases selected from the group consisting of depression; anxiety; addictions; obsessive compulsive disorder; affective neurosis; depressive neurosis; anxiety neurosis; dysthymic disorder; mood disorder; sexual dysfunction; psychosexual dysfunction; schizophrenia; manic depression; delirium; dementia; severe mental retardation and dyskinesias such as Huntington's disease and Tourette syndrome; diabetes; appetite/taste disorders; vomiting/nausea; asthma; Parkinson's disease; Cushing's syndrome/disease; basophil adenoma; prolactinoma; hyperprolactinemia; hypopituitarism; hypophysis tumour/adenoma; hypothalamic diseases; inflammatory bowel disease; gastric dyskinesia; gastric ulcers; Froehlich's syndrome; hypophysis diseases, hypothalamic hypogonadism; Kallman's syndrome (anosmia, hyposmia); functional or psychogenic amenorrhea; hypothalamic hypothyroidism; hypothalamic-adrenal dysfunction; idiopathic hyperprolactinemia; hypothalamic disorders of growth hormone deficiency; idiopathic growth deficiency; dwarfism; gigantism; acromegaly; disturbed biological and circadian rhythms; sleep disturbances associated with diseases such as neurological disorders, neuropathic pain and restless leg syndrome; heart and lung diseases, acute and congestive heart failure; hypotension; hypertension; urinary retention; osteoporosis; angina pectoris; myocardial infarction; ischemic or haemorrhagic stroke; subarachnoid haemorrhage; ulcers; allergies; benign prostatic hypertrophy; chronic renal failure; renal disease; impaired glucose tolerance; migraine; pain; enhanced or exaggerated sensitivity to pain such as hyperalgesia, causalgia, and allodynia; acute pain; burn pain; atypical facial pain; neuropathic pain; back pain; complex regional pain syndrome I and II; arthritic pain; sports injury pain; pain related to infection, HIV; post-chemotherapy pain; post-stroke pain; post-operative pain; neuralgia; conditions associated with visceral pain such as irritable bowel syndrome, migraine and angina; urinary bladder incontinence e.g. urge incontinence; tolerance to narcotics or withdrawal from narcotics; sleep disorders; eating disorders; cardiovascular disorders; neurodegenerative disorders; sleep apnea; narcolepsy; insomnia; parasomnia; and neurodegenerative disorders including nosological entities such as disinhibition-dementia-parkinsonism-amyotrophy complex; pallido-ponto-nigral degeneration epilepsy, seizure disorders and other diseases related to general orexin system dysfunctions.

13. Use according to claim 12 wherein said diseases are selected from the group consisting of eating disorders or sleep disorders.

14. Use according to claim 13 wherein said eating disorders comprise metabolic dysfunction, dysregulated appetite control, compulsive obesities, emeto-bulimia or anorexia nervosa.

15. Use according to claim 13 wherein said sleep disorders comprise insomnias, narcolepsy and other disorders of excessive sleepiness, sleep-related dystonias, restless leg syndrome, sleep apneas, jet-lag syndrome, shift-work syndrome, delayed or advanced sleep phase syndrome.

16. A method of treating or preventing diseases or disorders where an antagonist of human orexin receptors is required, which comprises administering to a subject in need thereof a therapeutically effective amount of a compound as claimed in claim 1, or a pharmaceutically acceptable salt thereof.

17. A process for the manufacture of pharmaceutical compositions for the treatment of disorders mentioned in claim 9, containing one or more compounds of claim 1, or a pharmaceutically acceptable salt or salts thereof, as active ingredients which process comprises mixing one or more active ingredient or ingredients with pharmaceutically acceptable excipients and adjuvants in a manner known per se.

18. Use of one or more compounds of claim 1 in combination with other pharmacologically active compounds comprising other orexin receptor antagonists, lipid lowering agents, anorectic agents, sleep inducing agents, antidepressants or other drugs beneficial for the prevention or treatment of disorders according to claim 9.

19. A process for the manufacture of pharmaceutical compositions for the treatment of disorders mentioned in claim 10, containing one or more compounds of claim 1, or a pharmaceutically acceptable salt or salts thereof, as active ingredients which process comprises mixing one or more active ingredient or ingredients with pharmaceutically acceptable excipients and adjuvants in a manner known per se.
